SEQUENCE LISTING (33 sequences)

SEQ ID NO: 1 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1278 aa, Clostridium botulinum
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKFCKSVIPRKGTKAPPRLCIRVNNRELFFVASESSYNENDINTPKEIDDTTNLNNNYRNNLDEVILDYNSETIPQISNQTLNTLVQDDSYVPRYDSNGTSEIEEHNVVDLNVFFYLHAQKVPEGETNISLTSSIDTALSEESQVYTFFSSEFINTINKPVHAALFISWINQVIRDFTTEATQKSTFDKIADISLVVPYVGLALNIGNEVQKENFKEAFELLGAGILLEFVPELLIPTILVFTIKSFIGSSENKNKIIKAINNSLMERETKWKEIYSWIVSNWLTRINTQFNKRKEQMYQALQNQVDAIKTVIEYKYNNYTSDERNRLESEYNINNIREELNKKVSLAMENIERFITESSIFYLMKLINEAKVSKLREYDEGVKEYLLDYISEHRSILGNSVQELNDLVTSTLNNSIPFELSSYTNDKILILYFNKLYKKIKDNSILDMRYENNKFIDISGYGSNISINGDVYIYSTNRNQFGIYSSKPSEVNIAQNNDIIYNGRYQNFSISFWVRIPKYFNKVNLNNEYTIIDCIRNNNSGWKISLNYNKIIWTLQDTAGNNQKLVFNYTQMISISDYINKWIFVTITNNRLGNSRIYINGNLIDEKSISNLGDIHVSDNILFKIVGCNDTRYVGIRYFKVFDTELGKTEIETLYSDEPDPSILKDFWGNYLLYNKRYYLLNLLRTDKSITQNSNFLNINQQRGVYQKPNIFSNTRLYTGVEVIIRKNGSTDISNTDNFVRKNDLAYINVVDRDVEYRLYADISIAKPEKIIKLIRTSNSNNSLGQIIVMDSIGNNCTMNFQNNNGGNIGLLGFHSNNLVASSWYYNNIRKNTSSNGCFWSFISKEHGWQEN

SEQ ID NO: 7 - protein, 1297 aa, Clostridium botulinum
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1285 aa, Clostridium botulinum
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGSDVEIIPQIERLQLREKALGHYKDIAKRLNNINKTIPSSWSSNIDKYKKIFSEKYNFDKDNTGNFVVNIDKFNSLYSDLTNVMSEVVYSSQYNVKNRTHYFSKHYLPVFANILDDNIYTIINGFNLTTKGFNIENSGQNIERNPALQKLSSESVVDLFTKVCLRLTRNSRDDSTCIQVKNNTLPYVADKDSISQEIFESQIITDETNVENYSDNFSLDESILDAKVPTNPEAVDPLLPNVNMEPLNVPGEEEVFYDDITKDVDYLNSYYYLEAQKLSNNVENITLTTSVEEALGYSNKIYTFLPSLAEKVNKGVQAGLFLNWANEVVEDFTTNIMKKDTLDKISDVSAIIPYIGPALNIGNSALRGNFKQAFATAGVAFLLEGFPEFTIPALGVFTFYSSIQEREKIIKTIENCLEQRVKRWKDSYQWMVSNWLSRITTQFNHISYQMYDSLSYQADAIKAKIDLEYKKYSGSDKENIKSQVENLKNSLDVKISEAMNNINKFIRECSVTYLFKNMLPKVIDELNKFDLKTKTELINLIDSHNIILVGEVDRLKAKVNESFENTIPFNIFSYTNNSLLKDMINEYFNSINDSKILSLQNKKNTLMDTSGYNAEVRVEGNVQLNPIFPFDFKLGSSGDDRGKVIVTQNENIVYNAMYESFSISFWIRINKWVSNLPGYTIIDSVKNNSGWSIGIISNFLVFTLKQNENSEQDINFSYDISKNAAGYNKWFFVTITTNMMGNMMIYINGKLIDTIKVKELTGINFSKTITFQMNKIPNTGLITSDSDNINMWIRDFYIFAKELDDKDINILFNSLQYTNVVKDYWGNDLRYDKEYYMINVNYMNRYMSKKGNGIVFNTRKNNNDFNEGYKIIIKRIIGNTNDTRVRGENVLYFNTTIDNKQYSLGMYKPSRNLGTDLVPLGALDQPMDEIRKYGSFIIQPCNTFDYYASQLFLSSNATTNRIGILSIGSYSFKLGDDYWFNHEYLIPVIKIEHYASLLESTSTHWVFVPASE

SEQ ID NO: 9 - protein, 1268 aa, Clostridium botulinum
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESKGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYDNLLSNYTAIASRLSQVNINNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLVVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLKAEYNIYSIKEELNKKVSLAMQNIDRFLTESSISYLMKLINEAKINKLSEYDKRVNQYLLNYILENSSTLGTSSVQELNNLVSNTLNNSIPFELSEYTNDKILISYFNRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 10 - protein, 1304 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 11 - protein, 1306 aa, Clostridium botulinum
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKNYLNNIDLEDKKTTSKTNVSYPCSLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYKNEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 12 - protein, 1315 aa, Clostridium tetani
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 13 - protein, 1296 aa, Clostridium botulinum [sequence omitted]

SEQ ID NO: 14 - DNA, 3888 bp, Clostridium botulinum
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 15 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 16 - protein, 1251 aa, Clostridium botulinum
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKIINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTFEDNRGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNCTMNFKNNNGNNIGLLGFKADTVVASTWYYTHMRDHTNSNGCFWNFISEEHGWQEK

SEQ ID NO: 17 - protein, 11 aa, Homo sapiens
RPKPQQFFGLM

SEQ ID NO: 18 - protein, 6 aa, Clostridium botulinum
NIINTS

SEQ ID NO: 19 - protein, 6 aa, Clostridium botulinum
NIVNTS

SEQ ID NO: 20 - protein, 6 aa, Clostridium botulinum
NIVNTS

SEQ ID NO: 21 - protein, 6 aa, Clostridium botulinum
NITNAS

SEQ ID NO: 22 - protein, 6 aa, Clostridium botulinum
NIINTS

SEQ ID NO: 23 - protein, 6 aa, Clostridium botulinum
NIINTS

SEQ ID NO: 24 - protein, 6 aa, Clostridium botulinum
NIINTS

SEQ ID NO: 25 - protein, 6 aa, Clostridium botulinum
NITNTS

SEQ ID NO: 26 - protein, 6 aa, Clostridium botulinum
EILNNI

SEQ ID NO: 27 - protein, 6 aa, Clostridium botulinum
EILNNI

SEQ ID NO: 28 - protein, 6 aa, Clostridium botulinum
EILNNI

SEQ ID NO: 29 - protein, 6 aa, Clostridium botulinum
EILNNI

SEQ ID NO: 30 - protein, 6 aa, Clostridium botulinum
DILNNI

SEQ ID NO: 31 - protein, 6 aa, Clostridium botulinum
EILNNI

SEQ ID NO: 32 - protein, 6 aa, Clostridium botulinum
EILNNI

SEQ ID NO: 33 - protein, 6 aa, Clostridium botulinum
EILNNI

(19) United States
(12) Patent Application Publication
(10) Pub. No.: US 2024/0016718 A1
(43) Pub. Date: Jan. 18, 2024

(54) TREATMENT OF SKIN CONDITIONS

(71) Applicant: IPSEN BIOPHARM LIMITED, Wrexham, EAW (GB)

(72) Inventors: Jacquie MAIGNEL, Wrexham (GB); Johannes KRUPP, Wrexham (GB)

(21) Appl. No.: 17/759,674

(22) PCT Filed: Jan. 29, 2021
(86) PCT No.: PCT/GB2021/050215

§ 371 Date: Jul. 28, 2022

(30) Foreign Application Priority Data
Jan. 31, 2020 (GB) 2001353.8

(51) Int. Cl. A61K 8/66; A61Q 19/08

(52) U.S. Cl. CPC A61K 8/66; A61Q 19/08; A61K 2800/91

(57) ABSTRACT

The present invention relates to the treatment of skin conditions with neurotoxins, and non-therapeutic uses for cosmetic treatment of the skin. Administration of the neurotoxin induces secretion of one or more sebaceous lipid(s) selected from a squalene, a fatty acid, a cholesterol, and a wax ester to an epidermal layer of skin.

### FIELD OF THE INVENTION

The present invention relates to the treatment of skin conditions with clostridial neurotoxins.

### BACKGROUND

Skin is the largest organ in the mammalian body, providing a barrier that protects the body against external irritation and infections as well as preventing water loss. The skin is composed of the epidermis (at the exterior of the skin), dermis and subcutis. The outermost layer of the epidermis, the stratum corneum (SC), is comprised of large, flattened, enucleated corneocytes and a lipid-enriched extracellular matrix. The major classes of lipids found in the SC include fatty acids, cholesterol, squalene and wax esters. The lipids are derived (e.g. excreted from) the sebaceous glands located within the skin, which excrete a mixture of such lipids in the form of sebum. These lipids are therefore referred to as sebaceous lipids.

The presence of these lipids is essential for the maintenance of a moist, pliable and healthy skin barrier. Indeed, healthy skin depends on an optimal lipid profile to form a barrier that confers protection and prevents excessive water loss, aids cell-cell communication and regulates cutaneous homeostasis and inflammation. Loss of lipids within the cutaneous lipid profile can have severe consequences for skin health and has been implicated in inflammatory skin conditions. Alteration of SC lipids composition leads to disrupted or impaired skin barrier functions and results in an increase in transepidermal water loss (TEWL). Increased TEWL has been observed in many skin diseases, such as atopic dermatitis (AD) and psoriasis. Thus, loss of these lipids (reducing the quality of the lipid profile of sebum) can cause, or predispose one to developing a skin condition.

When produced in excess, these lipids (in the form of sebum) can result in oily skin and acne. Therefore, currently available skin treatments (such as retinoid-based treatments) have focussed on reducing sebum level, at the expense of maintaining sufficient levels of skin-protective lipids, and associated side effects such as accelerated skin aging, skin irritation (e.g. redness), dehydration and infection. There is a comparative lack of treatments to increase/maintain key sebaceous lipid levels (e.g. toward maintaining a balanced level that allows protection and hydration of the skin, without causing acne or causing oily skin). Existing treatments (such as hormone treatment with hormones that influence sebum excretion from the sebaceous glands) typically have a systemic effect (not specific to the skin), and can thus cause undesirable off-target effects.

Accordingly, there is an increasing need for alternative/improved methods for treating skin conditions, in particular skin conditions resulting from insufficient sebaceous lipids (e.g. insufficient sebum quality).

The present invention addresses this problem by providing an alternative and/or improved means for inducing secretion/excretion of sebaceous lipids to the epidermis.

### SUMMARY

The present invention is predicated on the surprising finding that intradermal administration of a clostridial neurotoxin (such as a botulinum neurotoxin) leads to increased levels of key skin protective sebaceous lipids within sebum of the epidermis. The clostridial neurotoxin does not necessarily substantially change overall sebum levels, which is advantageous because increased sebum secretion is considered a major feature involved in the pathophysiology of acne. Thus, the invention leads to improved quality of the sebum, such that the sebum comprises a higher relative percentage of desirable lipids having both therapeutic and cosmetic applications.

This is surprising, as clostridial neurotoxins are understood to act by inhibiting secretion of molecules (e.g. by cleaving polypeptides involved in secretory vesicle docking to the cell membrane), rather than inducing or increasing secretion/excretion. For example, prior art methods involving subcutaneous administration of a botulinum toxin were reported to result in decreased sebum levels in the skin (which contrasts with the presently observed effect provided by intradermal administration, which increases levels of certain sebaceous lipids in the skin, e.g. without substantially changing overall sebum levels). The site of intradermal administration (between the epidermis and the dermis) is more peripheral than the site of subcutaneous administration (i.e the subcutaneous layer, below the dermis). The composition (e.g. chemical composition) of these layers differ, and it is hypothesised (although without wishing to be bound by theory) that a clostridial neurotoxin may act on the sebaceous gland via different modes of action depending on the layer of skin it is injected to, leading to different effects. The present inventors have now observed a hitherto unknown effect resulting from intradermal administration of a clostridial neurotoxin (e.g. BoNT/A), namely an induction (e.g. increase) in secretion/excretion of certain sebaceous lipids as alluded to above. Without wishing to be bound by theory, it is believed that a clostridial neurotoxin may interact (positively) with enzymes that catalyse production of these lipids (e.g. when administered intradermally).

Reference to the terms “key skin protective sebaceous lipids”, “desirable lipids”, and “certain sebaceous lipids” above means one or more of a squalene, a fatty acid, a cholesterol, and a wax ester, as described in more detail below.

### DETAILED DESCRIPTION

Broad aspects of the invention include any of:
a method for treating a skin condition, said method comprising administering to a patient a clostridial neurotoxin, preferably comprising administering intradermally to a patient a clostridial neurotoxin;
a clostridial neurotoxin for use in a method of treating a skin condition, said method comprising administering to a patient a clostridial neurotoxin, preferably comprising administering intradermally to a patient a clostridial neurotoxin;
use of a clostridial neurotoxin in the manufacture of a medicament to treat a skin condition in a patient, preferably for intradermal administration to a patient.

Broad aspects of the invention also include any of:
non-therapeutic use of a clostridial neurotoxin for cosmetic treatment of the skin, preferably comprising administering intradermally to a patient a clostridial neurotoxin;
non-therapeutic use of a clostridial neurotoxin for promoting rejuvenation of the skin, preferably comprising administering intradermally to a patient a clostridial neurotoxin;
a cosmetic method for (cosmetic) treatment of the skin, comprising administering to a patient a clostridial neurotoxin (preferably comprising administering intradermally to a patient a clostridial neurotoxin).

Following administration, the clostridial neurotoxin may induce secretion of one or more sebaceous lipid(s) to an epidermal layer of skin. In other words, the clostridial neurotoxin may treat the condition by inducing secretion of one or more sebaceous lipid(s) to an epidermal layer of skin.

Examples of such a sebaceous lipid(s) include squalene, a fatty acid, a cholesterol, and a wax ester. The skin condition is preferably a condition associated with a reduced level of a sebaceous lipid (e.g. one or more of squalene, a fatty acid, a cholesterol, and a wax ester) on an epidermal layer of the patient's skin, for example wherein said level of a sebaceous lipid is reduced relative to a level of the sebaceous lipid on a subject that does not have a skin condition. Sebaceous lipids involved in skin conditions, as well a examples of such conditions, are described in Sahle et. al. (Skin Pharmacol Physiol 2015; 28:42-55) which is incorporated herein by reference.

With regard to a non-therapeutic use and/or cosmetic method described herein, a cosmetic effect is preferably provided by increasing the level of a sebaceous lipid (e.g. one or more of squalene, a fatty acid, a cholesterol, and a wax ester) on an epidermal layer of the patient's skin, for example wherein said level of a sebaceous lipid is increased relative to a level of the sebaceous lipid on an epidermal layer of a subject that has not been administered the clostridial neurotoxin (or wherein said level of a sebaceous lipid is increased relative to a level of the sebaceous lipid on an epidermal layer of the patient pre-administration of the clostridial neurotoxin).

Preferably, following administration the clostridial neurotoxin induces:
secretion (e.g. excretion) of one or more sebaceous lipid(s) selected from a squalene, a fatty acid, a cholesterol, and a wax ester to an epidermal layer of skin; and/or
retention of a tachykinin peptide (such as Substance P) in dermis of the skin.

The therapeutic or cosmetic effect may be effected by inducing:
secretion (e.g. excretion) of one or more sebaceous lipid(s) selected from a squalene, a fatty acid, a cholesterol, and a wax ester to an epidermal layer of skin; and/or
retention of a tachykinin peptide (such as Substance P) in dermis of the skin;
   following administration of the clostridial neurotoxin to a patient.

Accordingly, the present invention provides a method for treating a skin condition, said method comprising administering intradermally to a patient a clostridial neurotoxin, wherein following administration the clostridial neurotoxin induces:
secretion (e.g. excretion) of one or more sebaceous lipid(s) selected from a squalene, a fatty acid, a cholesterol, and a wax ester to an epidermal layer of skin; and/or
retention of a tachykinin peptide (such as Substance P) in dermis of the skin.

An aspect of the invention provides a method for treating a skin condition by inducing:
secretion (e.g. excretion) of one or more sebaceous lipid(s) selected from a squalene, a fatty acid, a cholesterol, and a wax ester to an epidermal layer of skin; and/or
retention of a tachykinin peptide (such as Substance P) in dermis of the skin;
   following intradermal administration of the clostridial neurotoxin to a patient.

Another aspect of the invention provides a clostridial neurotoxin for use in a method of treating a skin condition, said method comprising administering intradermally to a patient a clostridial neurotoxin, wherein following administration the clostridial neurotoxin induces:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin.

An aspect of the invention provides a clostridial neurotoxin for use in a method of treating a skin condition by inducing:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin;
said method comprising administering intradermally to a patient a clostridial neurotoxin.

Advantageously, inducing secretion/excretion of a sebaceous lipid described herein to the epidermis can improve the appearance/youthfulness of the skin and allow for skin rejuvenation, such that invention also finds utility in the cosmetic field.

Thus, a further aspect of the invention provides non-therapeutic use of a clostridial neurotoxin for cosmetic treatment of the skin, wherein (preferably following intradermal administration to a patient) the clostridial neurotoxin induces:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin.

An aspect of the invention provides non-therapeutic use of a clostridial neurotoxin for cosmetic treatment of the skin by inducing:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin;
preferably wherein the method comprises administering intradermally to a patient a clostridial neurotoxin.

Preferably, a non-therapeutic use and/or cosmetic method of the invention comprises promoting rejuvenation of the skin by inducing secretion (e.g. excretion) of one or more sebaceous lipid(s) to an epidermal layer of skin; preferably wherein the one or more sebaceous lipid(s) is selected from a squalene, a fatty acid, a cholesterol, and a wax ester.

Another aspect of the invention provides non-therapeutic use of a clostridial neurotoxin for promoting rejuvenation of the skin by inducing:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin;
preferably wherein the method comprises administering intradermally to a patient a clostridial neurotoxin.

Another aspect provides a cosmetic method for (cosmetic) treatment of the skin, wherein (preferably following intradermal administration to a patient) the clostridial neurotoxin induces:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin.

An aspect of the invention provides a cosmetic method for (cosmetic) treatment of the skin by inducing:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin;
following administration of a clostridial neurotoxin to a patient (preferably following intradermal administration of a clostridial neurotoxin to a patient).

An aspect of the invention provides a cosmetic method for promoting rejuvenation of the skin by inducing:
secretion (e.g. excretion) of one or more sebaceous lipid(s) selected from a squalene, a fatty acid, a cholesterol, and a wax ester to an epidermal layer of skin; and/or
retention of a tachykinin peptide in dermis of the skin;
   following administration of a clostridial neurotoxin to a patient (preferably following intradermal administration of a clostridial neurotoxin to a patient).

A key advantage of employing a clostridial neurotoxin for therapy or cosmetic treatment is that the clostridial neurotoxin does not cause the undesirable side effects of existing skin treatments, such as retinoid-based creams. For example, the inventors have observed that intradermal injection of a botulinum neurotoxin does not cause erythema or scaling (e.g. peeling) of the skin, which is seen following the topical treatment of a common retinoid-based cream.

Various (optional) embodiments of the invention will now be described. It should be noted that each of the following embodiments may apply to any method, clostridial neurotoxin for use, or non-therapeutic use (e.g. cosmetic method) described herein.

In one embodiment, the epidermal layer (e.g. to which the sebaceous lipids are secreted) is one or more selected from the Stratum basale, the Stratum spinosum, the Stratum granulosum, or the Stratum corneum. In a preferable embodiment, the epidermal layer is the Stratum corneum.

Reference to “sebum” means an oily/waxy substance produced by the sebaceous glands of the skin, which typically serves to coat, moisturize, and protect the skin. Sebum is a complex mixture of fatty acids, sugars, waxes, and other natural chemicals that form a protective barrier against water evaporation. For example, sebum (e.g. human sebum) typically comprises fatty acids (˜57%, the main component of which are triglycerides), wax esters (˜26%), squalene (˜12%), and cholesterol (˜4.5%).

Without wishing to be bound by theory, it is believed that that the increase in certain sebum components (e.g. squalene, a fatty acid, a cholesterol, and a wax ester) observed following intradermal administration of a clostridial neurotoxin is due to a change in the composition (e.g. relative levels) of these sebum components, rather a total increase in the overall sebum level. That is, it has been observed that the relative levels of these lipids to the total (overall) sebum level may increase following intradermal administration of a clostridial neurotoxin.

Thus, advantageously, an unexpected technical effect of the present invention is an increase in the quality of sebum (e.g. by increasing the level of components which contribute to the beneficial properties of sebum on the skin). By improving the quality of sebum, rather than increasing total sebum levels, it is possible to treat a skin condition without inadvertently causing (or increasing the likelihood of) another skin condition such as oily skin and/or acne.

In one embodiment, the patient does not have oily skin (e.g. oily facial skin) prior to and/or subsequent to administration of the clostridial neurotoxin.

The term “oily skin” means a condition resulting from overproduction of sebum (e.g. from sebaceous glands under the skin's surface). A patient with oily skin may comprise sebum (e.g. on facial skin) at a level that is greater than 180 mg of sebum per cm² of skin (>180 mg/cm²). The sebum level (oily skin patient) may be 185 mg/cm², ≥190 mg/cm², or 195 mg/cm². Said level may refer to an average level of sebum (e.g. an average level on facial skin). Said level may preferably refer to a level (e.g. average level) of sebum present on a defined area of the face, such as the forehead, nasolabial sulcus, and/or nose.

As mentioned elsewhere herein, sebum (e.g. facial sebum) may be measured using a Sebumeter (SM 8151, CL-Electronics, Cologne, Germany).

Throughout this specification, any reference to a level of sebum may refer to a level of sebum as measurable with a Sebumeter.

In one embodiment, the patient does not have overproduction of sebum (e.g. total sebum level) subsequent to administration of the clostridial neurotoxin. In other words, in one embodiment, the patient does not suffer from overproduction of sebum (e.g. total sebum level) subsequent to administration of the clostridial neurotoxin.

In one embodiment, the patient has a sebum level of ≤180 mg/cm² of skin (e.g. facial skin). It may be said that such patient is a patient that does not have oily skin. Said level of sebum may refer to a level at an area of the patient's skin where the sebum is highest in the patient (e.g. as sebum levels tend to vary across different skin regions); for example, an area of the face, such as the forehead, nasolabial sulcus, and/or nose. Due the varying level of sebum across the skin, the patient may have sebum at 20 mg/cm² to ≤180 mg/cm² of skin (e.g. facial skin).

In one embodiment, the patient is a non-acneic patient.

The term “acneic” refers to the presence of acneic skin a patient (and thus “non-acneic” refers to the absence of acneic skin a patient). Acneic skin refers to skin having an excess of oil/sebum in the pores (e.g. typically occurring when the hormones of the body are over stimulated). This combined with bacteria and dead skin cell accumulation may lead to inflammation eruptions and severe breakouts. Some typical symptoms of acneic skin are: inflamed pustular breakouts (spots), severe blackheads and clogged pores, oiliness, coarser, thicker skin texture.

The treatment may be effected on the patient's face; for example at the forehead, nasolabial sulcus, and/or nose (bridge and/or apex). For example, the clostridial neurotoxin may be administered (e.g. intradermally administered) to the patient's face; for example to the forehead, nasolabial sulcus, and/or nose (bridge and/or apex).

In one embodiment, the treatment (e.g. induction of sebaceous lipid secretion) may be effected in a non-facial area of the skin. For example, the clostridial neurotoxin may be administered to a non-facial area of the patient skin (e.g. to non-facial skin). For example, the clostridial neurotoxin may be administered at a hand(s), foot, neck, scalp and/or back (e.g. hand(s), foot, and/or back). Areas of the back include the upper back and shoulders.

In one embodiment, following administration of the clostridial neurotoxin, no increase in the level (e.g. total level) of sebum on the skin (e.g. epidermal layer) of the patient is induced relative to a reference standard; wherein the reference standard corresponds to a level of sebum on the skin (e.g. epidermal layer) of a patient that has not been administered the clostridial neurotoxin (or wherein the reference standard corresponds to a level of sebum on the skin (e.g. epidermal layer) of the patient pre-administration of the clostridial neurotoxin).

Additionally or alternatively, following administration of the clostridial neurotoxin, no decrease in the level (e.g. total level) of sebum on the skin (e.g. epidermal layer) of the patient is induced relative to a reference standard; wherein the reference standard corresponds to a level of sebum on the skin (e.g. epidermal layer) of a patient that has not been administered the clostridial neurotoxin (or wherein the reference standard corresponds to a level of sebum on the skin (e.g. epidermal layer) of the patient pre-administration of the clostridial neurotoxin).

In other words, in one embodiment, following administration of the clostridial neurotoxin, the level of sebum on the skin (e.g. epidermal layer) of the patient is unchanged relative to a reference standard; wherein the reference standard corresponds to a level of sebum on the skin (e.g. epidermal layer) of a patient that has not been administered the clostridial neurotoxin (or wherein the reference standard corresponds to a level of sebum on the skin (e.g. epidermal layer) of the patient pre-administration of the clostridial neurotoxin).

The term “no decrease in the level of sebum on the skin” means that there is substantially no decrease in the level of sebum on the skin. The term “substantially” as used herein in the context of the term “no decrease in the level of sebum on the skin” preferably means there is no statistically significant decrease. Said decrease (which is not substantial) may be a decrease of less than 5%, 2%, 1% or 0.5%. More preferably, the term “no decrease in the level of sebum on the skin” as used herein means that the level of level of sebum on the skin (e.g. epidermal layer) is not decreased at all (i.e. the decrease in the level is 0%).

Similarly, the term “no increase in the level of sebum on the skin” means that there is substantially no increase in the level of sebum on the skin. The term “substantially” as used herein in the context of the term “no increase in the level of sebum on the skin” preferably means there is no statistically significant increase. Said increase (which is not substantial) may be an increase of less than 5%, 2%, 1% or 0.5%. More preferably, the term “no increase in the level of sebum on the skin” as used herein means that the level of level of sebum on the skin (e.g. epidermal layer) is not increased at all (i.e. the decrease in the level is 0%).

The term “the level of sebum on the skin (e.g. epidermal layer) of the patient is unchanged” means that there is substantially no change (e.g. no increase or decrease) in the level of sebum on the skin (e.g. epidermal layer). The term “substantially” as used herein in the context of the term “the level of sebum on the skin (e.g. epidermal layer) of the patient is unchanged” preferably means there is no statistically significant change. Said change (which is not substantial) may be a change of less than 5%, 2%, 1% or 0.5%. More preferably, the term “the level of sebum on the skin (e.g. epidermal layer) of the patient is unchanged” as used herein means that the level of level of sebum on the skin (e.g. epidermal layer) is not changed at all (i.e. the change in the level is 0%).

In one embodiment, following administration of the clostridial neurotoxin, a (the) level of sebum on the skin (e.g. epidermal layer) of the patient increases by <30%, <25%, <20%, <15%, <10%, <5%, or <1% (for example, <20%, <15%, <10%, <5%, or <1%). Preferably, following administration of the clostridial neurotoxin, a (the) level of sebum on the skin (e.g. epidermal layer) of the patient may increase by <10%. Said increase in the level of sebum may be compared relative to a reference standard—preferably wherein the reference standard corresponds to a level of sebum on the skin (e.g. epidermal layer) of a patient that has not been administered the clostridial neurotoxin. Additionally or alternatively, the reference standard may correspond to a level of sebum on the skin (e.g. epidermal layer) of the patient pre-administration of the clostridial neurotoxin.

In one embodiment, following administration of the clostridial neurotoxin, no increase in the level of erythema and/or scaling on the skin of the patient occurs relative to a reference standard; wherein the reference standard corresponds to a level of erythema and/or scaling on the skin of a patient (having said skin condition) that has not been administered the clostridial neurotoxin. Additionally or alternatively, the reference standard may correspond to a level of erythema and/or scaling on the skin of the patient pre-administration of the clostridial neurotoxin.

The term “no increase in the level of erythema and/or scaling on the skin” means that there is substantially no increase in the level of erythema and/or scaling on the skin. The term “substantially” as used herein in the context of the term “no increase in the level of erythema and/or scaling on the skin” preferably means there is no statistically significant increase. Said increase (which is not substantial) may be an increase of less than 5%, 2%, 1% or 0.5%. More preferably, the term “no increase in the level of erythema and/or scaling on the skin” as used herein means that the level of level of erythema and/or scaling on the skin is not increased at all (i.e. the decrease in the level is 0%).

The term “erythema” refers to a skin condition which presents as redness of the skin or mucous membranes, caused by hyperemia (increased blood flow) in superficial capillaries. It may occur due to skin injury, infection or inflammation.

The term “scaling” refers to the appearance of dry, cracked, or flaky skin. Also known as desquamation, scaling skin typically occurs when the outer layer of the skin, called the epidermis, begins to flake off. Scaling skin may arise when an injury or a medical condition damages the outer layer of skin.

Preferably, following administration of the clostridial neurotoxin, no change (e.g. no increase or decrease) occurs in the level of one or more of the following parameters:
sebaceous gland surface;
utriculi surface (utriculi being e.g. structures in the epidermis of the skin which are filled with solid impactions of horny cells resembling those of human open comedones);
back skin epidermis thickness;
dermis inflammation;
keratinocyte proliferation;
fibroblast proliferation;
sebaceous gland proliferation;
IL-alpha level in the dermis;
wherein the change is compared relative to the level of said one or more parameter in a reference standard; wherein the reference standard corresponds to a level of said one or more parameter in a subject/patient that has not been administered the clostridial neurotoxin. Additionally or alternatively, the reference standard may correspond to a level of said one or more parameter in the subject/patient pre-administration of the clostridial neurotoxin.

The “sebaceous lipids” are so-called because they are part of the sebum secreted by the sebaceous gland. “Sebaceous glands” are microscopic exocrine glands in the skin that secrete sebum (resembling an oily or waxy matter) to lubricate and waterproof the skin and hair of mammals. In humans, they occur in the greatest number on the face and scalp, but also on all parts of the skin except the palms of the hands and soles of the feet. The type of secretion of the sebaceous glands is referred to as holocrine.

The term “secretion” as used herein may be used interchangeably with the term “excretion”. This is because sebum (and thus sebaceous lipids) is produced by cells of the sebaceous gland called sebocytes, which lyse to release their contents of lipids/sebum (typically into the bottom of a hair follicle before the lipids/sebum slowly moves up the hair follicle to the skin surface). Thus, it may be considered that these cells ‘excrete’ the lipids/sebum to an epidermal layer. For example, the term “release” may be used to embrace secretion and/or excretion.

Throughout this specification, any reference to a level of a sebaceous lipid described herein preferably refers to a level of said sebaceous lipid at or close to the site of administration of the clostridial neurotoxin. For example, any reference to a level of a sebaceous lipid described herein may refer to a level of said sebaceous lipid (e.g. on the skin)≤1 cm, ≤2 cm, ≤4 cm, ≤6 cm, ≤8 cm, ≤10 cm, ≤12 cm, ≤16 cm, ≤18 cm or ≤20 cm from the site of administration of the clostridial neurotoxin. Reference to a level of a sebaceous lipid described herein may refer to a level of said sebaceous lipid ≤2 cm, ≤4 cm, ≤6 cm, ≤8 cm, or ≤10 cm (preferably ≤4 cm) from the site of administration of the clostridial neurotoxin.

An aspect of the present invention provides a method for treating a skin condition, said method comprising administering intradermally to a patient a clostridial neurotoxin, wherein following administration the clostridial neurotoxin induces:
release (e.g. secretion/excretion) of one or more sebaceous lipid(s) selected from a squalene, a fatty acid, a cholesterol, and a wax ester to an epidermal layer of skin.

An aspect of the invention provides a method for treating a skin condition by inducing:
release (e.g. secretion/excretion) of one or more sebaceous lipid(s) selected from a squalene, a fatty acid, a cholesterol, and a wax ester to an epidermal layer of skin
   following intradermal administration of the clostridial neurotoxin to a patient.

Another aspect of the invention provides a clostridial neurotoxin for use in a method of treating a skin condition, said method comprising administering intradermally to a patient a clostridial neurotoxin, wherein following administration the clostridial neurotoxin induces:
release (e.g. secretion/excretion) of one or more sebaceous lipid(s) selected from a squalene, a fatty acid, a cholesterol, and a wax ester to an epidermal layer of skin.

An aspect of the invention provides a clostridial neurotoxin for use in a method of treating a skin condition by inducing:
release (e.g. secretion/excretion) of one or more sebaceous lipid(s) selected from a squalene, a fatty acid, a cholesterol, and a wax ester to an epidermal layer of skin;
said method comprising administering intradermally to a patient the clostridial neurotoxin.

A further aspect of the invention provides non-therapeutic use of a clostridial neurotoxin for cosmetic treatment of the skin, wherein (preferably following intradermal administration to a patient) the clostridial neurotoxin induces:
release (e.g. secretion/excretion) of one or more sebaceous lipid(s) selected from a squalene, a fatty acid, a cholesterol, and a wax ester to an epidermal layer of skin.

A further aspect of the invention provides non-therapeutic use of a clostridial neurotoxin for promoting rejuvenation of the skin, wherein (preferably following intradermal administration to a patient) the clostridial neurotoxin induces:
release (e.g. secretion/excretion) of one or more sebaceous lipid(s) selected from a squalene, a fatty acid, a cholesterol, and a wax ester to an epidermal layer of skin.

A further aspect of the invention provides non-therapeutic use of a clostridial neurotoxin for cosmetic treatment of the skin by inducing:
release (e.g. secretion/excretion) of one or more sebaceous lipid(s) selected from a squalene, a fatty acid, a cholesterol, and a wax ester to an epidermal layer of skin,
following administration of the clostridial neurotoxin to the patient (preferably following intradermal administration of the clostridial neurotoxin to the patient).

A further aspect of the invention provides non-therapeutic use of a clostridial neurotoxin for promoting rejuvenation of the skin by inducing:
release (e.g. secretion/excretion) of one or more sebaceous lipid(s) selected from a squalene, a fatty acid, a cholesterol, and a wax ester to an epidermal layer of skin,
following administration of the clostridial neurotoxin to the patient (preferably following intradermal administration of the clostridial neurotoxin to the patient).

Another aspect provides a cosmetic method for (cosmetic) treatment of the skin, wherein (preferably following intradermal administration to a patient) the clostridial neurotoxin induces:
release (e.g. secretion/excretion) of one or more sebaceous lipid(s) selected from a squalene, a fatty acid, a cholesterol, and a wax ester to an epidermal layer of skin.

Another aspect provides a cosmetic method for promoting rejuvenation of the skin, wherein (preferably following intradermal administration to a patient) the clostridial neurotoxin induces:
release (e.g. secretion/excretion) of one or more sebaceous lipid(s) selected from a squalene, a fatty acid, a cholesterol, and a wax ester to an epidermal layer of skin.

Another aspect provides a cosmetic method for (cosmetic) treatment of the skin, by inducing:
release (e.g. secretion/excretion) of one or more sebaceous lipid(s) selected from a squalene, a fatty acid, a cholesterol, and a wax ester to an epidermal layer of skin,
following administration of the clostridial neurotoxin to the patient (preferably following intradermal administration of the clostridial neurotoxin to the patient).

Another aspect provides a cosmetic method for promoting rejuvenation of the skin, by inducing:
release (e.g. secretion/excretion) of one or more sebaceous lipid(s) selected from a squalene, a fatty acid, a cholesterol, and a wax ester to an epidermal layer of skin,
following administration of the clostridial neurotoxin to the patient (preferably following intradermal administration of the clostridial neurotoxin to the patient).

The term “release” as used herein encompasses secretion of a sebaceous lipid as well as excretion of a sebaceous lipid. For example, the term “release” as used herein may mean excretion (of a sebaceous lipid). Additionally or alternatively, the term “release” as used herein may mean secretion (of a sebaceous lipid).

The terms “release”, “secretion” and “excretion” of the sebaceous lipid(s) mean release/secretion/excretion (respectively) from a sebaceous gland (e.g. sebocyte) of the patient.

The term “treat” or “treating” as used herein encompasses prophylactic treatment (e.g. to prevent onset of a skin condition) as well as corrective treatment (treatment of a patient already suffering from skin condition). In a preferable embodiment, the term “treat” or “treating” as used herein means corrective treatment. The term “treat” or “treating” encompasses treating both the skin condition and a symptom thereof. In some embodiments the term “treat” or “treating” refers to a symptom of a skin condition.

Therefore, a clostridial neurotoxin may be administered to a patient in a therapeutically effective amount or a prophylactically effective amount.

A “therapeutically effective amount” is any amount of the clostridial neurotoxin which when administered alone or in combination to a patient for treating a skin condition (or a symptom thereof) is sufficient to effect such treatment of the skin condition, or symptom thereof.

A “prophylactically effective amount” is any amount of the clostridial neurotoxin that, when administered alone or in combination to a patient inhibits or delays the onset or reoccurrence of a skin condition (or a symptom thereof). In some embodiments, the prophylactically effective amount prevents the onset or reoccurrence of a skin condition entirely. “Inhibiting” the onset means either lessening the likelihood of skin condition onset (or symptom thereof), or preventing the onset entirely.

Preferably, a therapeutically and/or prophylactically effective amount is an amount which does not lead to muscle paralysis. The term “muscle paralysis” preferably refers to long-term muscle paralysis, since transient muscle paralysis may occur for a short period following administration.

The terms “subject”, “individual” and “patient” are used interchangeably herein to refer to a mammalian subject. In one embodiment the “patient” is a human, a companion animal (e.g. a pet such as a dog, cat, and/or rabbit), livestock (e.g. a pig, sheep, cattle, and/or a goat), and/or a horse. In a preferable embodiment, the subject (patient) is a human.

The term “non-therapeutic use” refers to use of the clostridial neurotoxin for cosmetic purposes as opposed to therapeutic application. The term “cosmetic method” refers to a method in which the clostridial neurotoxin is administered for cosmetic purposes as opposed to therapeutic application. In aspects and embodiments related to a “non-therapeutic” use of the invention, the subject (e.g. to whom the clostridial neurotoxin is administered) preferably does not have a skin condition, for example the subject may not have a skin condition described herein. The non-therapeutic use is preferably to improve the aesthetic appearance (e.g. youthfulness) of the skin, e.g. in older subjects, noting that endogenous sebaceous lipid secretion typically decreases with age. In aspects and embodiments related to a “cosmetic method” of the invention, the subject (e.g. to whom the clostridial neurotoxin is administered) preferably does not have a skin condition, for example the subject may not have a skin condition described herein. The cosmetic method is preferably to improve the aesthetic appearance (e.g. youthfulness) of the skin, e.g. in older subjects, nothing that endogenous sebaceous lipid secretion typically decreases with age.

In methods of the invention, the patient may not have been previously diagnosed as having a skin condition. Alternatively, the patient may have been previously diagnosed as having a skin condition. The patient may also be one who exhibits disease risk factors, or one who is asymptomatic for a skin condition. The patient may also be one who is suffering from or is at risk of developing a skin condition. In one embodiment, the patient has been previously administered a therapy for a skin condition.

The skin condition can be any skin condition that may be alleviated (or prevented) due the presence of higher quality sebum on the skin. “Higher quality sebum” level of one or sebaceous lipid (e.g. one or more sebaceous lipid selected from a squalene, a fatty acid, a cholesterol and a wax ester) in sebum is increased.

Preferably, the skin condition to be treated is associated with the presence of a reduced level of the one or more sebaceous lipid (e.g. one or more sebaceous lipid selected from a squalene, a fatty acid, a cholesterol and a wax ester) on an epidermal layer of the patient, compared to a level of the one or sebaceous lipid on a epidermal layer of a healthy patient (e.g. a patient that does not have a skin condition).

Preferably, the skin condition is caused by a reduced level of said one or more sebaceous lipid (e.g. one or more sebaceous lipid selected from a squalene, a fatty acid, a cholesterol and a wax ester), on an epidermal layer of the patient, compared to a level of said one or more sebaceous lipid on an epidermal layer of a subject that does not have the skin condition.

Preferably, the skin condition is a skin condition that is suppressed (e.g. treated or prevented) by the presence of one or more sebaceous lipid(s) selected from a squalene, a fatty acid, a cholesterol, and a wax ester on an epidermal layer of skin.

In one embodiment, the clostridial neurotoxin is administered to a patient having sebum that comprises fatty acid at a concentration of ≤50% (v/v), ≤45% (v/v), ≤40% (v/v), ≤35% (v/v), ≤30% (v/v), ≤25% (v/v), ≤20% (v/v), ≤15% (v/v), or ≤10% (v/v). In some embodiments, the clostridial neurotoxin is administered to a patient having sebum that comprises fatty acid at a concentration of ≤45% (v/v), ≤40% (v/v), ≤35% (v/v), ≤30% (v/v), ≤25% (v/v). The clostridial neurotoxin may be administered to a patient having sebum that comprises fatty acid at a concentration of ≤45% (v/v). Preferably, the clostridial neurotoxin may be administered to a patient having sebum that comprises fatty acid at a concentration of ≤40% (v/v).

In one embodiment, the clostridial neurotoxin is administered to a patient having sebum that comprises wax ester at a concentration of ≤20% (v/v), ≤15% (v/v), ≤10% (v/v), or ≤5% (v/v). In some embodiments, the clostridial neurotoxin is administered to a patient having sebum that comprises fatty acid at a concentration of ≤15% (v/v), ≤10% (v/v), or ≤5% (v/v). The clostridial neurotoxin may be administered to a patient having sebum that comprises wax ester at a concentration of ≤15% (v/v). Preferably, the clostridial neurotoxin may be administered to a patient having sebum that comprises wax ester at a concentration of ≤10% (v/v).

In one embodiment, the clostridial neurotoxin is administered to a patient having sebum that comprises squalene at a concentration of ≤10% (v/v), ≤8% (v/v), ≤6% (v/v), ≤4% (v/v), or ≤2% (v/v). In some embodiments, the clostridial neurotoxin is administered to a patient having sebum that comprises squalene at a concentration of ≤8% (v/v), ≤6% (v/v), or ≤4% (v/v). The clostridial neurotoxin may be administered to a patient having sebum that comprises squalene at a concentration of ≤8% (v/v). Preferably, the clostridial neurotoxin may be administered to a patient having sebum that comprises squalene at a concentration of ≤6% (v/v).

In one embodiment, the clostridial neurotoxin is administered to a patient having sebum that comprises cholesterol at a concentration of ≤4% (v/v), ≤3% (v/v), ≤2% (v/v), or ≤1% (v/v). In some embodiments, the clostridial neurotoxin is administered to a patient having sebum that comprises cholesterol at a concentration of ≤3% (v/v), ≤2% (v/v), or ≤1% (v/v). The clostridial neurotoxin may be administered to a patient having sebum that comprises cholesterol at a concentration of ≤3% (v/v). Preferably, the clostridial neurotoxin may be administered to a patient having sebum that comprises cholesterol at a concentration of ≤2% (v/v).

In one embodiment, the skin condition is a disorder associated with aberrant secretion of a sebaceous lipid (e.g. one or more sebaceous lipid selected from a squalene, a fatty acid, a cholesterol and a wax ester) to an epidermal layer of the patient. Preferably, the patient has said sebaceous lipid on an epidermal layer at a lower level than a subject that does not have the skin condition. In other words, in one embodiment, the level of said one or more sebaceous lipid on an epidermal layer (e.g. Stratum corneum) of the patient (pre-administration of the clostridial neurotoxin) is lower than a level of said one or more sebaceous lipid on an epidermal layer of a subject that does not have the skin condition (e.g. preferably at least 5%, 10%, 15% or 20% lower; more preferably at least 10% lower).

By way of example, conditions such as xerosis, psoriasis, atopic dermatitis, and ichthyosis may be associated with the depletion of such sebaceous lipids.

In one embodiment, the skin condition is one or more selected from acne (e.g. acne vulgaris), atopic dermatitis, netherton syndrome, psoriasis, pruritis, dehydrated skin (e.g. dry or cracked skin), actinic keratosis, rosacea, carbuncle, eczema (e.g. atopic eczema), cellulitis, dermatitis, skin cancer, keratosis pilaris, skin aging (e.g. normal and/or premature skin aging), burn, scaring, type 2 Gaucher disease, Sjögren-Larsson syndrome, lamellar ichthyosis, X-linked ichthyosis, bullous ichthyosiform erythroderma, essential free fatty acid (FFA) deficiency, aged dry skin and hypohidrotic ectodermal dysplasia.

In one embodiment, the skin condition is one or more selected from acne, atopic dermatitis, netherton syndrome, psoriasis, dehydrated skin (e.g. dry or cracked skin), actinic keratosis, rosacea, carbuncle, eczema (e.g. atopic eczema), cellulitis, dermatitis, skin cancer, keratosis pilaris, skin aging (e.g. normal and/or premature skin aging), burn or scaring. For example, the skin condition may be one or more selected from acne, atopic dermatitis, dermatitis, psoriasis, eczema or dehydrated skin (e.g. dry or cracked skin). Additionally or alternatively, the skin condition may be one or more selected from dermatitis, psoriasis, or eczema.

In one embodiment, the skin condition is one or more selected from acne (e.g. acne vulgaris), atopic dermatitis, psoriasis, pruritis, xerosis, dehydrated skin (e.g. dry or cracked skin), eczema (e.g. atopic eczema), type 2 Gaucher disease, Sjögren-Larsson syndrome, lamellar ichthyosis, X-linked ichthyosis, bullous ichthyosiform erythroderma, essential FFA deficiency, aged dry skin and hypohidrotic ectodermal dysplasia. For example, the skin condition may be one or more selected from atopic dermatitis, psoriasis, xerosis, dehydrated skin (e.g. dry or cracked skin), eczema (e.g. atopic eczema), type 2 Gaucher disease, Sjögren-Larsson syndrome, lamellar ichthyosis, X-linked ichthyosis, bullous ichthyosiform erythroderma, essential FFA deficiency, aged dry skin and hypohidrotic ectodermal dysplasia.

Preferably, the skin condition may be one or more selected from xerosis, psoriasis, atopic dermatitis, and ichthyosis (more preferably xerosis).

Additionally or alternatively, the clostridial neurotoxin may be administered to a patient that has been exposed to one or more factors (preferably external factors) that affect the composition of sebaceous lipids, for example a chemical used for cleansing and sanitation purposes, an environmental pollutant, and/or a pharmaceutical ingredient. The term “affect the composition of sebaceous lipids” means reduces the level a sebaceous lipid (e.g. one or more sebaceous lipid selected from a squalene, a fatty acid, a cholesterol and a wax ester) on an epidermal layer of the patient.

Advantageously, the clostridial neurotoxin may be administered subsequent to (or prior to, or simultaneously with) an alternative (e.g. first) skin treatment that may decrease the quality of sebum on the patient's skin, which, for example, predisposes (or potentially predisposes) the patient to developing a skin condition as a side effect. The term “decrease the quality of sebum on the patient's skin” preferably refers to a decreased in the level of a sebaceous lipid (e.g. one or more sebaceous lipid selected from a squalene, a fatty acid, a cholesterol and a wax ester) on an epidermal layer of the patient.

For example, the clostridial neurotoxin may be administered subsequent to (or prior to, or simultaneously with) a laser treatment, a chemical peel and/or acne treatment (e.g. adapalene treatment, which typically reduces sebum levels), preferably enhancing recovery of the skin following said alternative treatment.

In one embodiment, the clostridial neurotoxin is administered to a patient whose skin has been exposed to an agent that causes a decrease in the level of said one or more sebaceous lipid (e.g. selected from squalene, a fatty acid, a cholesterol, and a wax ester), on an epidermal layer of the patient, compared to a level of said one or more sebaceous lipid on an epidermal layer of a subject that has not been exposed to the agent.

The clostridial neurotoxin may be administered to a patient whose skin has been exposed to a chemical pollutant, for example that may act as a contact allergen or irritants causing allergic or nonallergic contact dermatitis. Examples of such chemicals include metal(s), soap(s), fragrance(s), preservative(s), botanical(s) and paraphenylenediamine.

The clostridial neurotoxin may be administered to a patient whose skin has been exposed to elevated heat (e.g. ≥30° C.) and/or moisture, for example a level of heat and/or moisture that may affect the composition of sebaceous lipids. Such elevated heat and/or moisture may lead to increased sweating causing water loss, skin dryness and pruritus.

In one embodiment, the patient is a patient which has received (or will receive) a treatment (e.g. skin treatment) that reduces the level of one or more sebaceous lipid (e.g. one or more sebaceous lipid selected from a squalene, a fatty acid, a cholesterol and a wax ester) on an epidermal layer of the skin; for example reduced relative to the level of said one or more sebaceous lipid in a reference standard; wherein the reference standard corresponds to a level of said one or more sebaceous lipid on an epidermal layer of a patient that has not received said treatment (e.g. skin treatment), or wherein the reference standard corresponds to a level of said one or more sebaceous lipid on an epidermal layer of the patient prior to receiving said treatment (e.g. skin treatment). Examples of such treatment may include laser treatment, a chemical peel and/or acne treatment (e.g. adapalene treatment).

The term “one or more sebaceous lipid(s)” preferably means at least 2, 3, or 4 of the indicated sebaceous lipid(s) (e.g. selected from a squalene, a fatty acid, a cholesterol, and a wax ester) are induced following administration of a clostridial neurotoxin. Thus, secretion of at least 2, 3, or 4 sebaceous lipid(s) may be induced by a clostridial neurotoxin. More preferably, the term “one or more sebaceous lipid(s)” means secretion of all of the indicated sebaceous lipid(s) (e.g. squalene, a fatty acid, a cholesterol, and a wax ester) are induced following administration of a clostridial neurotoxin.

In one embodiment, the sebaceous lipid (the “one or more sebaceous lipid”) is a fatty acid; and one or more (e.g. two or more, three or more, or four or more) selected from a squalene, a cholesterol, and a wax ester. In one embodiment, the sebaceous lipid is a squalene; and one or more (e.g. two or more, three or more, or four or more) selected from a fatty acid, a cholesterol, and a wax ester. In one embodiment, the sebaceous lipid is a cholesterol; and one or more (e.g. two or more, three or more, or four or more) selected from a fatty acid, a squalene, and a wax ester. In one embodiment, the sebaceous lipid is a wax ester; and one or more (e.g. two or more, three or more, or four or more) selected from a fatty acid, a squalene, and a cholesterol.

In one embodiment, the sebaceous lipid is a fatty acid. Examples of fatty acids include free fatty acid(s), diglyceride(s) (e.g. a glyceride composed of two fatty acid chains that are covalently bound to a single glycerol molecule via an ester linkage), triglyceride(s) (e.g. an ester derived from glycerol and three fatty acid chains), ceramide(s) and cholesteryl ester(s). For example, the fatty acid may be a triglyceride and/or a free fatty acid. The fatty acid may preferably be a triglyceride.

The free fatty acid may comprise (e.g. with one or more substitutions) or consist of the following formula:

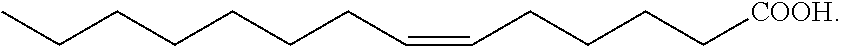 [SMILES: CCCCCCC/C=C\CCCCC(=O)O]

The free fatty acid may comprise (e.g. with one or more substitutions) or consist of the following formula:

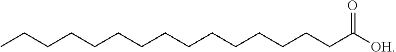 [SMILES: CCCCCCCCCCCCCCCC(=O)O]

The triglyceride may comprise or consist of the following formula:

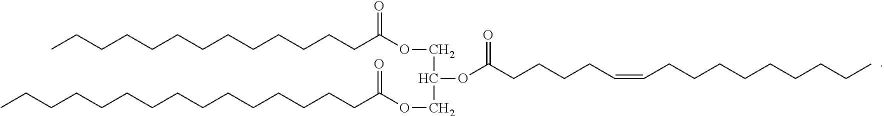 [SMILES: CCCCCCCCC/C=C\CCCCC(=O)OC(COC(=O)CCCCCCCCCCCCC)COC(=O)CCCCCCCCCCCCCCC]

Many fatty acids produced by the sebaceous gland are associated with two unique features, namely that they are branched-chain fatty acids, and that they are highly unsaturated. Taken together with the triglycerides, they account for the predominant proportion (e.g. ˜57%) of sebum. The fatty acids of sebum are believed to be (amongst other things) important for allowing delivery of fat-soluble anti-oxidants to the skin surface and pro- and anti-inflammatory activity exerted by certain molecules. In one embodiment, the fatty acid may be a branched-chain fatty acid. Additionally or alternatively (preferably additionally), the fatty acid may be a highly unsaturated fatty acid.

Preferable examples of fatty acids include linoleic acid (a polyunsaturated omega-6 fatty acid), and Sapienic acid (e.g. cis-6 hexadecenoic).

A sebaceous lipid (e.g. fatty acid) may be defined by reference to a retention time on a gas chromatography (GC) column. “Retention time” (RT) is a measure of the time (preferably in minutes) taken for a solute (e.g. lipid) to pass through a chromatography column, where a sample comprising the analyte is subjected to gas chromatography analysis (preferably gas chromatography with flame-ionization detection). It is calculated as the time from injection (into the GC column) of a sample (e.g. in the present application, sebum/sebaceous lipids) to detection of the analyte. Said detection is typically by way of a defined peak on a gas chromatogram output (with the area of said peak allowing detection of the level of the sebaceous lipid).

Gas chromatography (GC) analysis may be performed with a GC/flame-ionization detection (FID) apparatus, such as 7890B (Agilent); and optionally with acquisition performed with Empower, version 3471 (Waters).

Said GC analysis may comprise:
helium carrier gas;
a GC column having dimensions of 30 m×0.25 mm×0.1 μm;
an injector temperature of about 350° C., split mode injection;
a GC column temperature gradient of:
   80° C.-240° C. at 10° C./minute(s);
   240° C.-320° C. at 5° C./minute(s);
   320° C.-350° C. at 2° C./minute(s); and
   350° C. for 20 minutes;
a flame-ionization detection (FID) detector temperature of 250° C.;
a FID hydrogen flow of 35 mL/minute;
a FID air flow of 350 mL/minute; and/or
a FID make up helium flow of 25 mL/minute.

Preferably, said GC analysis comprises subjecting 1 μl of sebum to GC analysis with a helium carrier gas (35 cm/sec), and a GC column comprising 5% Phenyl, 95% Dimethylpolysiloxane and having dimensions of 30 m×0.25 mm×0.1 μm (e.g. GC of Zebron ZB-SHT);
wherein injection is by split mode injection (e.g. 4 mm i.d. liner, straight, deactivated, no packing)
wherein a column temperature gradient comprises:
   80° C. to 240° C. at 10° C./min
   240° C. to 320° C. at 5° C./min
   320° C. to 350° C. at 2° C./min
   350° C. for 20 min
wherein lipid detection is performed with a flame ionization detector (FID) demonstrating:
   detector temperature 250° C.
   a FID Hydrogen flow of 35 ml/min
   a FID Air flow of 350 ml/min
   a FID Make up Helium flow of 25 ml/min.

In one embodiment, the fatty acid is a fatty acid having a retention time of about 15.5-16 minutes (e.g. about 15.7 or 15.8 minutes), about 20-20.2 minutes (e.g. about 20 minutes) and/or about 22-22.2 minutes (e.g. about 22.1 minutes)—e.g. on a GC column. Preferably, the fatty acid may be a fatty acid having a retention time of about 22-22.2 minutes (e.g. on a GC column); more preferably about 22.1 minutes.

A retention time may be expressed in absolute terms or in relative terms (relative to a reference/standard analyte). For example, the fatty acid may be defined by reference to a “relative retention time” (RRT) on a gas chromatography column. RRT is an expression of an analyte's (e.g. lipid's) retention time relative to a reference/standard analyte's retention time (e.g. RRT=standard RT/sample RT). The analyte and the reference/standard analyte are preferably subjected to GC analysis simultaneously. For example, a sample (e.g. sebaceous lipid sample) which is subjected to GC analysis may comprise both the analyte (e.g. fatty acid) and the reference/standard analyte.

The reference/standard analyte may preferably be, for example, squalene.

For example, compare the retention time for a fatty acid (FA4) to the retention time of squalene (ST1) on the gas chromatogram output in FIG. 17A (an exploded view is provided in FIG. 17B). Here, it can be seen that the retention time for squalene is about 25 minutes, and the retention time for fatty acid (FA4) is 22.1 minutes. This demonstrates that a RTT (relative to a squalene retention time) for the fatty acid could be: 25 minutes/22.1 minutes=−1.13 minutes.

In one embodiment, the fatty acid is a fatty acid having a relative retention time of:
about 1.5-1.6 minutes (e.g. about 1.6 or 1.58 minutes) relative to a squalene retention time;
about 1.23-1.25 minutes (e.g. about 1.25 minutes) relative to a squalene retention time; and/or
about 0.9-1.14 minutes (e.g. about 1.13 minutes) relative to a squalene retention time;
preferably wherein the retention time (e.g. relative retention time) is determinable by gas chromatography.

The fatty acid may be a fatty acid having a relative retention time of 1-1.15 minutes (e.g. about 1.13 minutes) relative to a squalene retention time (e.g. as determinable by gas chromatography).

Preferably, the fatty acid may be a fatty acid having a relative retention time of 0.9-1.14 minutes (e.g. about 1.13 minutes) relative to a squalene retention time (e.g. as determinable by gas chromatography); more preferably about 1.12-1.14 minutes (e.g. about 1.13 minutes) relative to a squalene retention time (e.g. as determinable by gas chromatography).

The term “fatty acid” embraces ceramides (which are typically composed of a fatty acid and a sphingosine). For example, increasing the level of fatty acids/ceramides such as ceramide 1 linoleate can be beneficial in the treatment of skin conditions including atopic dermatitis. Ceramide 1 linoleate is the main repository of epidermal linoleic acid and changes in its levels are associated with cutaneous abnormalities such as atopic dermatitis. It is also subject to dramatic seasonal variation and decreases with age, such that a treatment of the present invention may be used to combat these seasonal and age-related changes.

A ceramide may comprise (e.g. having one or more additional substitutions) or consist of the following formula:

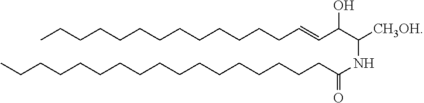

The term “fatty acid” as used herein may also embrace a “cholesteryl ester”, which is an ester of cholesterol, wherein an ester bond is formed between the carboxylate group of a fatty acid and the hydroxyl group of cholesterol.

A cholesteryl ester may be a cholesteryl ester having a retention time of about 40-42 minutes (e.g. about 41 minutes)—e.g. on a GC column.

The cholesteryl ester may be a cholesteryl ester having a relative retention time of 0.55-0.65 minutes, 0.57-0.63 minutes, or 0.59-0.61 minutes (e.g. about 0.6 minutes) relative to a squalene retention time (e.g. as determinable by gas chromatography).

Preferably, the cholesteryl ester may be a cholesteryl ester having a relative retention time of 0.59-0.625 minutes (e.g. about 0.6 minutes) relative to a squalene retention time (e.g. as determinable by gas chromatography).

The cholesteryl ester may comprise (e.g. having one or more additional substitutions) or consist of the following formula:

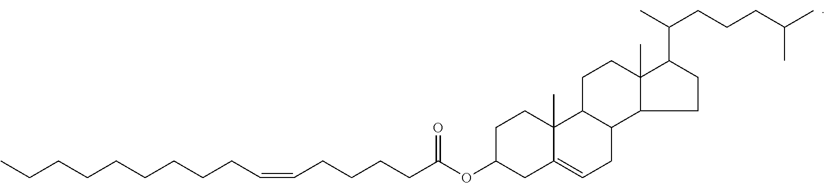 [SMILES: CCCCCCCCC/C=C\CCCCC(=O)OC1CCC2(C)C(=CCC3C2CCC2(C)C(C(C)CCCC(C)C)CCC32)C1]

In one embodiment, the sebaceous lipid is a wax ester.

The wax esters are typically the second most abundant lipid in sebum, representing about 26% of (normal) sebum in humans. A “wax ester” is an ester of a fatty acid and a fatty alcohol (e.g. wax esters are formed by combining one fatty acid with one fatty alcohol). Wax esters are important for reducing evaporative water loss from the skin, and thus skin dehydration.

In one embodiment, the wax ester is a wax ester having a retention time of about 30-32 minutes (e.g. about 31 minutes), about 35-36 minutes (e.g. about 35.5 minutes) and/or about 37-39 minutes (e.g. about 38 minutes)—e.g. on a GC column.

Preferably, the wax ester may be a wax ester having a relative retention time of 0.78-0.83 minutes (e.g. about 0.8 minutes), 0.69-0.71 minutes (e.g. 0.7 minutes), and/or about 0.64-0.67 minutes (e.g. about 0.74 minutes)—relative to a squalene retention time (e.g. as determinable by gas chromatography).

The wax ester may comprise or consist of the following formula:

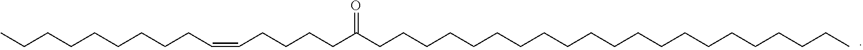 [SMILES: CCCCCCCCC/C=C\CCCCC(=O)CCCCCCCCCCCCCCCCCCCCC]

In one embodiment, the sebaceous lipid is a squalene.

Squalene (as referred to in the present disclosure) preferably comprises (e.g. having one or more additional substitutions) or consists of the following structure:

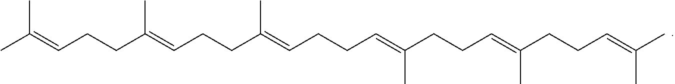 [SMILES: CC(C)=CCC/C(C)=C/CC/C(C)=C/CC/C=C(\C)CC/C=C(\C)CCC=C(C)C]

Squalenes are unique to sebum (i.e. they are not found in any other secretion of the body), and typically represent about 12% of (normal) sebum in humans. Squalene is a linear intermediate preceding cholesterol in its biosynthesis. In more detail, in the sebaceous gland, the squalene produced is not converted into lanosterol, halting the completion of the biosynthetic pathway leading to cholesterol. Possible explanations for the squalene build-up in the sebaceous gland have been hypothesised to include an overexpression/increase in the activity of squalene-synthase in the cells; or decreased level/activity of the enzymes involved in the conversion of squalene to cholesterol. Without wishing to be bound by theory, it is hypothesised that intradermal administration of a clostridial neurotoxin may positively interact with one or more of the mechanisms that lead to squalene build-up in the sebaceous gland.

Squalene, as one of the most important hydrating agents in nature, helps to maintain hydration of the most superficial layer of epidermis. Moreover, squalene possesses antioxidant properties against solar radiation and, consequently, skin cancer, since it reduces the production of free radicals at the level of sun-exposed skin. It has been reported that shark liver oil, which is rich in squalene and alkylglycerol, is protective against bacterial and fungal infections, particularly in patients with atopic dermatitis and xerosis-related skin lesions. When topically administered (e.g. in an exogenous/commercial formulation), squalene rapidly and effectively penetrates into skin and provides healthy elasticity and regaining of flexibility.

Said squalene may have a retention time of about 24-26 minutes (e.g. about 25 minutes)—e.g. on a GC column.

In one embodiment, the sebaceous lipid is cholesterol.

Cholesterol (as referred to in the present disclosure) preferably comprises (e.g. having one or more additional substitutions) or consists of the following structure:

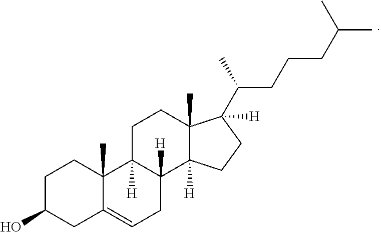

Cholesterol is the least abundant lipid in sebum, accounting (together with its esters) for about 2-4.5% of total sebum lipids in humans. By increasing the level of sebum cholesterol, the invention may allow for combatting conditions associated with age-related decreases in cholesterol in the skin. The levels of natural cholesterol in the skin start decreasing dramatically from the mid-fifties, inducing breaking of the lipid barrier. This results in increased dehydration, increasing skin cracking and the potential for infection.

Said cholesterol may have a retention time of about 24.2-24.6 minutes (e.g. about 24.4 minutes)—e.g. on a GC column.

The cholesterol may have a relative retention time of 0.95-1.1 minutes, 0.97-1.07 minutes, 1-1.05 minutes, or 1.01-1.03 minutes (e.g. about 1.02 minutes) relative to a squalene retention time (e.g. as determinable by gas chromatography).

Preferably, the cholesterol may have a relative retention time of 1.01-1.03 minutes (e.g. about 1.02 minutes) relative to a squalene retention time (e.g. as determinable by gas chromatography).

The terms “inducing the secretion of”, “inducing the excretion of” and “inducing the release of”, in the context of said one or more sebaceous lipid(s) means the level of one or more sebaceous lipid(s) in sebum is increased. Preferable levels of such increase are outlined below, together with references/controls against which such increase can be quantified. For the avoidance of doubt, the following embodiments remain intended to relate to any method of treatment, clostridial neurotoxin for use, non-therapeutic use and cosmetic method described herein.

The term “promoting the rejuvenation of skin”, is preferably quantified by a corresponding increase in the level of one or more sebaceous lipid(s) (e.g. selected from a squalene, a fatty acid, a cholesterol, and a wax ester) in sebum. For example, the term “rejuvenation of skin” preferably refers to promoting the youthfulness/youthful appearance of skin via the effects of sebaceous lipid(s) described herein e.g. effects including improved skin hydration, elasticity etc. Thus, the following embodiment, outlining levels of sebaceous lipid increase, may be combined with any non-therapeutic use and/or cosmetic method herein to quantify a promotion in rejuvenation of the skin. For example, where the level of one or more sebaceous lipid(s) in sebum is increased by at least 10%, it may be said that rejuvenation of the skin has been increased by a corresponding level (10%).

In one embodiment, following administration of the clostridial neurotoxin, the level of one or more sebaceous lipid(s) (e.g. selected from a squalene, a fatty acid, a cholesterol, and a wax ester) in sebum (e.g. the patient's sebum) may increase. For example, the level of one or more sebaceous lipid(s) (e.g. selected from a squalene, a fatty acid, a cholesterol, and a wax ester) in sebum (e.g. the patient's sebum) may increase by at least 10%, 20%, 30%, 40%, 50%, 60%, 70%, 80%, 90% or 100% (preferably at least 60%). Preferably, said increase is an increase compared to a level of said one or more sebaceous lipid in sebum of a subject that has not been administered the clostridial neurotoxin. Additionally or alternatively, said increase may be an increase compared to the level of said one or more sebaceous lipid in sebum (e.g. the patient's sebum) of the patient pre-administration of the clostridial neurotoxin.

For example, subsequent to administration of a clostridial neurotoxin according to the invention:
the level of a fatty acid in sebum (e.g. the patient's sebum) may increase by at least 10%, 20%, 30%, 40%, 50%, 60%, 70%, 80%, 90% or 100% (preferably at least 60%);
the level of a wax ester in sebum (e.g. the patient's sebum) may increase by at least 10%, 20%, 30%, 40%, 50%, 60%, 70%, 80%, 90% or 100% (preferably at least 60%);
the level of squalene in sebum (e.g. the patient's sebum) may increase by at least 10%, 20%, 30%, 40%, 50%, 60%, 70%, 80%, 90% or 100% (preferably at least 60%); and/or
the level of a cholesterol in sebum (e.g. the patient's sebum) may increase by at least 10%, 20%, 30%, 40%, 50%, 60%, 70%, 80%, 90% or 100% (preferably at least 60%).

Preferably, said increase is an increase compared to a level of said one or more sebaceous lipid in sebum of a subject that has not been administered the clostridial neurotoxin. Additionally or alternatively, said increase may be an increase compared to the level of said one or more sebaceous lipid in sebum (e.g. the patient's sebum) of the patient pre-administration of the clostridial neurotoxin.

In one embodiment, subsequent to administration of a clostridial neurotoxin, the level of a fatty acid in sebum (e.g. the patient's sebum) is increased by at least 10%, 20%, 30%, 40%, 50%, 60%, 70%, 80%, 90%, 100%, 110%, 120% or 130%. In some embodiments, subsequent to administration of a clostridial neurotoxin, the level of a fatty acid in sebum (e.g. the patient's sebum) is increased by at least 20%, 30%, 40%, 50%, 60%, or 70%. For example, subsequent to administration of a clostridial neurotoxin, the level of a fatty acid in sebum (e.g. the patient's sebum) may be increased by at least 10%. Preferably, the level of a fatty acid in sebum (e.g. the patient's sebum) may be increased by at least 50%. Said increase in the level of a fatty acid in sebum (e.g. the patient's sebum) may be an increase compared to a level of a fatty acid in sebum of a subject that has not been administered the clostridial neurotoxin. Additionally or alternatively, said increase in the level of a fatty acid in sebum (e.g. the patient's sebum) may be an increase compared to the level of a fatty acid in sebum of the patient pre-administration of the clostridial neurotoxin. Throughout this specific, comparisons of lipid levels preferably refer to a comparison at the site of administration of the clostridial neurotoxin.

In one embodiment, subsequent to administration of a clostridial neurotoxin, the level of a wax ester in sebum (e.g. the patient's sebum) is increased by at least 10%, 20%, 30%, 40%, 50%, 60%, 70%, 80%, 90%, 100%, 110%, 120% or 130%. In some embodiments, subsequent to administration of a clostridial neurotoxin, the level of a wax ester in sebum (e.g. the patient's sebum) is increased by at least 20%, 30%, 40%, 50%, 60%, or 70%. For example, subsequent to administration of a clostridial neurotoxin, the level of a wax ester in sebum (e.g. the patient's sebum) may be increased by at least 10%. Preferably, the level of a wax ester in sebum (e.g. the patient's sebum) may be increased by at least 50%. Said increase in the level of a wax ester in sebum (e.g. the patient's sebum) may be an increase compared to the level of a wax ester in sebum of a subject that has not been administered the clostridial neurotoxin. Additionally or alternatively, said increase in the level of a wax ester in sebum (e.g. the patient's sebum) may be an increase compared to the level of a wax ester in sebum of the patient pre-administration of the clostridial neurotoxin. Throughout this specific, comparisons of lipid levels preferably refer to a comparison at the site of administration of the clostridial neurotoxin.

In one embodiment, subsequent to administration of a clostridial neurotoxin, the level of squalene in sebum (e.g. the patient's sebum) is increased by at least 10%, 20%, 30%, 40%, 50%, 60%, 70%, 80%, 90%, 100%, 110%, 120% or 130%. In some embodiments, subsequent to administration of a clostridial neurotoxin, the level of squalene in sebum (e.g. the patient's sebum) is increased by at least 20%, 30%, 40%, 50%, 60%, or 70%. In some embodiments, subsequent to administration of a clostridial neurotoxin, the level of squalene in sebum (e.g. the patient's sebum) is increased by at least 80%, 90%, 100%, 110%, 120%, 130%, 140%, 150% or 160%. For example, subsequent to administration of a clostridial neurotoxin, the level of squalene in sebum (e.g. the patient's sebum) may be increased by at least 10%. Preferably, the level of a squalene in sebum (e.g. the patient's sebum) may be increased by at least 50%. Said increase in the level of squalene in sebum (e.g. the patient's sebum) may be an increase compared to the level of squalene in sebum of a subject that has not been administered the clostridial neurotoxin. Additionally or alternatively, said increase in the level of squalene in sebum (e.g. the patient's sebum) may be an increase compared to the level of squalene in sebum of the patient pre-administration of the clostridial neurotoxin. Throughout this specific, comparisons of lipid levels preferably refer to a comparison at the site of administration of the clostridial neurotoxin.

In one embodiment, subsequent to administration of a clostridial neurotoxin, the level of a cholesterol in sebum (e.g. the patient's sebum) is increased by at least 10%, 20%, 30%, 40%, 50%, 60%, 70%, 80%, 90% or 100%, 110%, 120% or 130%. In some embodiments, subsequent to administration of a clostridial neurotoxin, the level of a cholesterol in sebum (e.g. the patient's sebum) is increased by at least 20%, 30%, 40%, 50%, 60%, or 70%. In some embodiments, subsequent to administration of a clostridial neurotoxin, the level of a cholesterol in sebum (e.g. the patient's sebum) is increased by at least 80%, 90%, 100%, 110%, 120%, 130%, 140%, 150% or 160%. For example, subsequent to administration of a clostridial neurotoxin, the level of a cholesterol in sebum (e.g. the patient's sebum) may be increased by at least 10%. Preferably, the level of a cholesterol in sebum (e.g. the patient's sebum) may be increased by at least 50%. Said increase in the level of a cholesterol in sebum (e.g. the patient's sebum) may be an increase compared to the level of a cholesterol in sebum of a subject that has not been administered the clostridial neurotoxin. Additionally or alternatively, said increase in the level of a cholesterol in sebum (e.g. the patient's sebum) may be an increase compared to the level of a cholesterol in sebum of the patient pre-administration of the clostridial neurotoxin. Throughout this specific, comparisons of lipid levels preferably refer to a comparison at the site of administration of the clostridial neurotoxin.

Administration of a clostridial neurotoxin may increase the proportion of sebum which is contributed to by a fatty acid, wax ester, cholesterol and/or squalene.

In one embodiment, subsequent to administration of a clostridial neurotoxin according to the invention:
the relative proportion of a fatty acid in sebum (e.g. the patient's sebum) may be (e.g. may increase from typical values such as 57%) at least about 60%, 65%, 70%, 75% or 80%;
the relative proportion of a wax ester in sebum (e.g. the patient's sebum) may be (e.g. may increase from typical values such as 26%) at least about 30%, 35%, 40%, 45% or 50%;
the relative proportion of squalene in sebum (e.g. the patient's sebum) may be (e.g. may increase from typical values such as 12%) at least about 15%, 20%, 25%, 30%, 35% or 40%; and/or
the relative proportion of a cholesterol in sebum (e.g. the patient's sebum) may be (e.g. may increase from typical values such as 4.5%) at least about 5%, 10%, 15%, 20% or 25%.

For example, subsequent to administration of a clostridial neurotoxin according to the invention:
the relative proportion of a fatty acid in sebum (e.g. the patient's sebum) may be (e.g. may increase from typical values such as 57%) about 60%, 65%, 70%, 75% or 80%;
the relative proportion of a wax ester in sebum (e.g. the patient's sebum) may be (e.g. may increase from typical values such as 26%) about 30%, 35%, 40%, 45% or 50%;
the relative proportion of squalene in sebum (e.g. the patient's sebum) may be (e.g. may increase from typical values such as 12%) about 15%, 20%, 25%, 30%, 35% or 40%; and/or
the relative proportion of a cholesterol in sebum (e.g. the patient's sebum) may be (e.g. may increase from typical values such as 4.5%) about 5%, 10%, 15%, 20% or 25%.

The term “relative proportion” means the total volume of the sebaceous lipid in the sebum relative to the total volume of sebum. The relative proportion is preferably measured as % volume of sebaceous lipid over volume of sebum (v/v).

Comparisons of said “relative proportion” may be with reference to sebum in the vicinity of the administration site of the clostridial neurotoxin e.g. within less than or equal to 5 cm, 4 cm, 3 cm, 2 cm, or 1 cm of the administration site.

In one embodiment, subsequent to administration of a clostridial neurotoxin, the relative proportion of a fatty acid in sebum (e.g. the patient's sebum) may be at least about 60%, 65%, 70%, 75% or 80%. In some embodiments, subsequent to administration of a clostridial neurotoxin, the relative proportion of a fatty acid in sebum (e.g. the patient's sebum) may be at least about 65%, 70%, 75% or 80%. For example, subsequent to administration of a clostridial neurotoxin, the relative proportion of a fatty acid in sebum (e.g. the patient's sebum) may be at least about 70%. Said relative proportion of a fatty acid in sebum may be an increase from a typical value (e.g. basal value) such as about 57%.

In one embodiment, subsequent to administration of a clostridial neurotoxin, the relative proportion of a wax ester in sebum (e.g. the patient's sebum) may be at least about 30%, 35%, 40%, 45% or 50%. In some embodiments, subsequent to administration of a clostridial neurotoxin, the relative proportion of a wax ester in sebum (e.g. the patient's sebum) may be at least about 35%, 40%, 45% or 50%. For example, subsequent to administration of a clostridial neurotoxin, the relative proportion of a wax ester in sebum (e.g. the patient's sebum) may be at least about 40%. Said relative proportion of a wax ester in sebum may be an increase from a typical value (e.g. basal value) such as about 26%.

In one embodiment, subsequent to administration of a clostridial neurotoxin, the relative proportion of squalene in sebum (e.g. the patient's sebum) may be at least about 15%, 20%, 25%, 30%, 35% or 40%. In some embodiments, subsequent to administration of a clostridial neurotoxin, the relative proportion of squalene in sebum (e.g. the patient's sebum) may be at least about 20%, 25%, 30%, 35% or 40%. For example, subsequent to administration of a clostridial neurotoxin, the relative proportion of squalene in sebum (e.g. the patient's sebum) may be at least about 25%. Said relative proportion of squalene in sebum may be an increase from a typical value (e.g. basal value) such as about 12%.

In one embodiment, subsequent to administration of a clostridial neurotoxin, the relative proportion of a cholesterol in sebum (e.g. the patient's sebum) may be at least about 5%, 10%, or 15%. In one embodiment, subsequent to administration of a clostridial neurotoxin, the relative proportion of a cholesterol in sebum (e.g. the patient's sebum) may be at least about 5%, 10%, 15%, 20% or 25%. In one embodiment, subsequent to administration of a clostridial neurotoxin, the relative proportion of a cholesterol in sebum (e.g. the patient's sebum) may be at least about 10%, 15%, 20% or 25%. For example, subsequent to administration of a clostridial neurotoxin, the relative proportion of a cholesterol in sebum (e.g. the patient's sebum) may be at least about 10%. Said relative proportion of a cholesterol in sebum may be an increase from a typical value (e.g. basal value) such as about 4.5%.

In one embodiment, following administration of the clostridial neurotoxin, the relative proportion of one or more sebaceous lipid(s) (e.g. selected from a squalene, a fatty acid, a cholesterol, and a wax ester) in sebum (e.g. the patient's sebum) may increase. For example, the relative proportion of one or more sebaceous lipid(s) (e.g. selected from a squalene, a fatty acid, a cholesterol, and a wax ester) in sebum (e.g. the patient's sebum) may increase by at least 10%, 20%, 30%, 40%, 50%, 60%, 70%, 80%, 90% or 100% (preferably at least 60%). Preferably, said increase is an increase compared to the relative proportion of said one or more sebaceous lipid in sebum (e.g. the patient's sebum) of a patient that has not been administered the clostridial neurotoxin. Additionally or alternatively, said increase may be an increase compared to the relative proportion of said one or more sebaceous lipid in sebum (e.g. the patient's sebum) of the patient pre-administration of the clostridial neurotoxin.

For example, subsequent to administration of a clostridial neurotoxin according to the invention:
the relative proportion of a fatty acid in sebum (e.g. the patient's sebum) may increase by at least 10%, 20%, 30%, 40%, 50%, 60%, 70%, 80%, 90% or 100% (preferably at least 60%);
the relative proportion of a wax ester in sebum (e.g. the patient's sebum) may increase by at least 10%, 20%, 30%, 40%, 50%, 60%, 70%, 80%, 90% or 100% (preferably at least 60%);
the relative proportion of squalene in sebum (e.g. the patient's sebum) may increase by at least 10%, 20%, 30%, 40%, 50%, 60%, 70%, 80%, 90% or 100% (preferably at least 60%); and/or
the relative proportion of a cholesterol in sebum (e.g. the patient's sebum) may increase by at least 10%, 20%, 30%, 40%, 50%, 60%, 70%, 80%, 90% or 100% (preferably at least 60%).

In one embodiment, subsequent to administration of a clostridial neurotoxin, the relative proportion of a fatty acid in sebum (e.g. the patient's sebum) is increased by at least 10%, 20%, 30%, 40%, 50%, 60%, 70%, 80%, 90%, 100%, 110%, 120% or 130%. In some embodiments, subsequent to administration of a clostridial neurotoxin, the relative proportion of a fatty acid in sebum (e.g. the patient's sebum) is increased by at least 20%, 30%, 40%, 50%, 60%, or 70%. For example, subsequent to administration of a clostridial neurotoxin, the relative proportion of a fatty acid in sebum (e.g. the patient's sebum) may be increased by at least 10%. Preferably, the relative proportion of a fatty acid in sebum (e.g. the patient's sebum) may be increased by at least 50%. Said increase in the relative proportion of a fatty acid in sebum (e.g. the patient's sebum) may be an increase compared to the relative proportion of a fatty acid in sebum of a subject that has not been administered the clostridial neurotoxin. Additionally or alternatively, said increase in the relative proportion of a fatty acid in sebum (e.g. the patient's sebum) may be an increase compared to the relative proportion of a fatty acid in sebum of the patient pre-administration of the clostridial neurotoxin.

In one embodiment, subsequent to administration of a clostridial neurotoxin, the relative proportion of a wax ester in sebum (e.g. the patient's sebum) is increased by at least 10%, 20%, 30%, 40%, 50%, 60%, 70%, 80%, 90%, 100%, 110%, 120% or 130%. In some embodiments, subsequent to administration of a clostridial neurotoxin, the relative proportion of a wax ester in sebum (e.g. the patient's sebum) is increased by at least 20%, 30%, 40%, 50%, 60%, or 70%. For example, subsequent to administration of a clostridial neurotoxin, the relative proportion of a wax ester in sebum (e.g. the patient's sebum) may be increased by at least 10%. Preferably, the relative proportion of a wax ester in sebum (e.g. the patient's sebum) may be increased by at least 50%. Said increase in the relative proportion of a wax ester in sebum (e.g. the patient's sebum) may be an increase compared to the relative proportion of a wax ester in sebum of a subject that has not been administered the clostridial neurotoxin. Additionally or alternatively, said increase in the relative proportion of a wax ester in sebum (e.g. the patient's sebum) may be an increase compared to the relative proportion of a wax ester in sebum of the patient pre-administration of the clostridial neurotoxin.

In one embodiment, subsequent to administration of a clostridial neurotoxin, the relative proportion of squalene in sebum (e.g. the patient's sebum) is increased by at least 10%, 20%, 30%, 40%, 50%, 60%, 70%, 80%, 90%, 100%, 110%, 120% or 130%. In some embodiments, subsequent to administration of a clostridial neurotoxin, the relative proportion of squalene in sebum (e.g. the patient's sebum) is increased by at least 20%, 30%, 40%, 50%, 60%, or 70%. In some embodiments, subsequent to administration of a clostridial neurotoxin, the relative proportion of squalene in sebum (e.g. the patient's sebum) is increased by at least 80%, 90%, 100%, 110%, 120%, 130%, 140%, 150% or 160%. For example, subsequent to administration of a clostridial neurotoxin, the relative proportion of squalene in sebum (e.g. the patient's sebum) may be increased by at least 10%. Preferably, the relative proportion of a squalene in sebum (e.g. the patient's sebum) may be increased by at least 50%. Said increase in the relative proportion of squalene in sebum (e.g. the patient's sebum) may be an increase compared to the relative proportion of squalene in sebum of a subject that has not been administered the clostridial neurotoxin. Additionally or alternatively, said increase in the relative proportion of squalene in sebum (e.g. the patient's sebum) may be an increase compared to the relative proportion of squalene in sebum of the patient pre-administration of the clostridial neurotoxin.

In one embodiment, subsequent to administration of a clostridial neurotoxin, the relative proportion of a cholesterol in sebum (e.g. the patient's sebum) is increased by at least 10%, 20%, 30%, 40%, 50%, 60%, 70%, 80%, 90% or 100%, 110%, 120% or 130%. In some embodiments, subsequent to administration of a clostridial neurotoxin, the relative proportion of a cholesterol in sebum (e.g. the patient's sebum) is increased by at least 20%, 30%, 40%, 50%, 60%, or 70%. In some embodiments, subsequent to administration of a clostridial neurotoxin, the relative proportion of a cholesterol in sebum (e.g. the patient's sebum) is increased by at least 80%, 90%, 100%, 110%, 120%, 130%, 140%, 150% or 160%. For example, subsequent to administration of a clostridial neurotoxin, the relative proportion of a cholesterol in sebum (e.g. the patient's sebum) may be increased by at least 10%. Preferably, the relative proportion of a cholesterol in sebum (e.g. the patient's sebum) may be increased by at least 50%. Said increase in the relative proportion of a cholesterol in sebum (e.g. the patient's sebum) may be an increase compared to the relative proportion of a cholesterol in sebum of a subject that has not been administered the clostridial neurotoxin. Additionally or alternatively, said increase in the relative proportion of a cholesterol in sebum (e.g. the patient's sebum) may be an increase compared to the relative proportion of a cholesterol in sebum of the patient pre-administration of the clostridial neurotoxin.

An induction of sebaceous lipid secretion (subsequent to administration of clostridial neurotoxin) may be measured by detecting an increase in the level of the sebaceous lipid on the skin surface (e.g. epidermal layer) of a patient. The increase is preferably measured relative to a reference standard that has not been administered with the clostridial neurotoxin.

An increase in sebaceous lipids may be determined by any suitable method known to the person skilled in the art, with particular reference to the Examples described herein. For example, a lipid level may be measured by subjecting a sebum sample to gas chromatography analysis and generating a chromatogram output; and detecting the area of one or more defined peak in said chromatogram, wherein said one or more defined peak corresponds to a fatty acid, a wax ester, squalene and/or cholesterol. The area of said one or more defined peak provides for detection of the level of the corresponding lipid in the sebum. In such methods, the sebum sample may be collected (e.g. have been collected) by contacting a collection means with a surface of the skin. Examples of such collection means include a cotton pad, glass cone (or cylinder), glass disk, paper and/or a hydrophobic film. Preferably, the collection means is a glass disk.

Preferably, the “sebum sample” is a lipid extraction, comprising lipids extracted (e.g. purified) from sebum. Suitable methods for lipid extraction are described in the Examples.

Alternatively or additionally, immunological methods may be used to measure the levels of a molecule described herein. For example, the level of a tachykinin peptide (e.g. Substance P) may be measured by subjecting an isolated skin sample to immunological analysis, such as ELISA analysis.

Tachykinin peptides are neuropeptides typically from ten to twelve amino acid residues long, the best known of which is Substance P. The genes that produce tachykinin peptides encode precursor proteins called preprotachykinins, which are processed into smaller peptides (e.g. tachykinin peptides) by posttranslational proteolytic processing. The genes also code for multiple splice forms that are made up of different sets of peptides. Tachykinin peptides are involved in inflammation.

In one embodiment, subsequent to administration, the clostridial neurotoxin may induce retention of a tachykinin peptide (preferably Substance P) in dermis of the skin. In one embodiment, the tachykinin peptide is one or more selected from Substance P, neurokinin A, neuropeptide K, or neuropeptide gamma (preferably Substance P).

In one embodiment, following administration, the clostridial neurotoxin may induce secretion (e.g. excretion) of one or more sebaceous lipid(s) selected from a squalene, a fatty acid, a cholesterol, and a wax ester to an epidermal layer of skin; and induce retention of a tachykinin peptide (preferably Substance P) in dermis of the skin.

“Substance P” is peptide that is a member of the tachykinin neuropeptide family. It is a neuropeptide, acting as a neurotransmitter and as a neuromodulator. Substance P and its closely related neurokinin A (NKA) are produced from a polyprotein precursor after differential splicing of the preprotachykinin A gene.

Substance P as referred to herein may comprise an amino acid sequence having at least 80%, 85%, 90%, 95% or 100% (e.g. at least 95%) sequence identity to the sequence of SEQ ID NO.: 17. Preferably, Substance P as referred to herein may comprise or consist of an amino acid sequence of SEQ ID NO.: 17.

It may be particularly advantageous to both induce lipid secretion as well as tachykinin peptide (preferably Substance P) retention. The depletion or disturbance of the major sebaceous lipids in the skin/Stratum corneum (which can be caused by various environmental and physical factors such as soap, dry air and age) is one of the etiological factors producing dryness and barrier disruption in skin conditions. As a result, the skin loses water and becomes dry, cracked and fissured and allows the entrance of allergens, toxins and microorganisms that can inflame and irritate the skin. The inflammation, in return, may cause further disruption of the barrier function. This may be mitigated by either inducing sebaceous lipid secretion or tachykinin peptide retention by a method of the invention, and indeed by inducing both lipid secretion and tachykinin peptide retention. The invention may thus be preventive of loss in the barrier function of sebaceous lipids, which can lead to skin conditions such as severe dryness, itching and scratching that can further lead to secondary skin infections such as herpes, molluscum, warts, staphylococcus, streptococcus, pseudomonas, fungus, yeast and tuberculosis.

The induction of sebaceous lipid secretion (and/or decrease in erythema/scaling) may be measured in a human patient/subject (e.g. the analysis may be performed on an isolated sebum sample obtained from a human patient/subject).

For convenience, the induction of sebaceous lipid secretion (and/or decrease in erythema/scaling) may be measured in a suitable model organism, such as a mouse.

A particularly suitable model organism is the (Hairless) Rhino Mouse (harbouring the mutation hr^{rh-J}). Rhino mice have spleen cells with a defective response to T-dependent antigens mice, and have increased numbers of Thy1 positive epidermal dendritic cells. As a result, Rhino mice are hairless by 5 weeks and their skin becomes wrinkled and thickened. Rhino mice are now a widely accepted model organism for the study of skin conditions (and the effect of therapeutic/cosmetic agents on such skin conditions). Importantly, Rhino mice continue to produce sebum (e.g. at effectively wild-type levels), which can be readily collected from the skin surface (e.g. via a glass cone) due to the absence of hair on the mice.

Further details of the clostridial neurotoxins embraced by the invention are provided below, together with technological background information.

Bacteria in the genus Clostridia produce highly potent and specific protein toxins, which can poison neurons and other cells to which they are delivered. Examples of such clostridial toxins include the neurotoxins produced by C. tetani (TeNT) and by C. botulinum (BoNT) serotypes A-G, as well as those produced by C. baratii and C. butyricum.

Clostridial neurotoxins cause muscle paralysis by inhibiting cholinergic transmission in the peripheral nervous system, in particular at the neuromuscular junction, and can thus be lethal. In nature, clostridial neurotoxins are synthesised as a single-chain polypeptide that is modified post-translationally by a proteolytic cleavage event to form two polypeptide chains joined together by a disulphide bond. Cleavage occurs at a specific cleavage site, often referred to as the activation site, which is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the H_{N} domain.

The mode of action of clostridial neurotoxins relies on five distinct steps: (1) binding of the H_{C} domain to the cell membrane of its target neuron, followed by (2) internalisation of the bound toxin into the cell via an endosome, (3) translocation of the L-chain by the H_{N} domain across the endosomal membrane and into the cytosol, (4) proteolytic cleavage of intracellular transport proteins known as SNARE proteins by the L-chain which provides a non-cytotoxic protease function, and (5) inhibition of cellular secretion from the target cell.

In this cascade of events, SNARE proteins (Soluble N-ethylmaleimide-Sensitive Factor Attachment protein REceptor) are integral to intracellular vesicle fusion, and thus to secretion of molecules via vesicle transport from a cell. Examples of SNARE proteins present in neurons include, among others, SNAP-25, VAMP, or Syntaxin. The non-cytotoxic protease function of the L-chain is, on the other hand, a zinc-dependent endopeptidase activity, which exhibits a high substrate specificity for SNARE proteins. Accordingly, once delivered to a neuronal target cell, the non-cytotoxic protease of clostridial neurotoxins, by cleaving its substrate SNARE protein, inhibits neurotransmitter release, which consequently leads to neuroparalysis. Thus, the clostridial neurotoxins act by suppressing secretion.

Thanks to their unique properties, Clostridial neurotoxins, such as botulinum toxin, have been successfully employed in a wide range of therapeutic applications, in particular for motor and autonomic disorders, to restore for example the activity of hyperactive nerve endings to normal levels. At least seven antigenically distinct BoNTs serotypes have been described so far, namely BoNT/A, BoNT/B, BoNT/C, BoNT/D, BoNT/E, BoNT/F, BoNT/G (Rossetto, O. et al., “Botulinum neurotoxins: genetic, structural and mechanistic insights.” Nature Reviews Microbiology 12.8 (2014): 535-549). In addition to the seven major serotypes, naturally occurring chimeric serotypes, like BoNT/D-C, also exist (Moriishi et al., “Mosaic structures of neurotoxins produced from Clostridium botulinum types C and D organisms”. Biochim Biophys Acta. (1996); 1307:123-126.).

Furthermore, recently an eighth serotype, BoNT/X, has been identified (Zhang, et al. “Identification and characterization of a novel botulinum neurotoxin”. Nature Communications, vol 8, Article number: 14130 (2017)). The term “clostridial neurotoxin” may also embrace newly discovered botulinum neurotoxin protein family members expressed by non-clostridial microorganisms, such as the Enterococcus encoded toxin which has closest sequence identity to BoNT/X, the Weissella oryzae encoded toxin called BoNT/Wo (NCBI Ref Seq: WP_027699549.1), which cleaves VAMP2 at W89-W90, the Enterococcus faecium encoded toxin (GenBank: OTO22244.1), which cleaves VAMP2 and SNAP25, and the Chryseobacterium pipero encoded toxin (NCBI Ref.Seq: WP_034687872.1).

Despite this diversity, BoNT/A remains the serotype of choice in therapy, with three commonly available commercial preparations (Botox®, Dysport® and Xeomin®), while only one BoNT/B product is available on the market (Neurobloc®/Myobloc®). To this day, these BoNT/A and BoNT/B products, which are toxins purified from clostridial strains, are the only two BoNT serotypes that are currently approved by regulatory agencies for use in humans for applications ranging, among others, from spasticity, bladder dysfunction, or hyperhidrosis (for BoNT/A) (see for example: https://www.medicines.org.uk/emc/medicine/112,https://www.medicines.org.uk/e mc/medicine/870, https://www.medicines.org.uk/emc/medicine/2162, herein incorporated by reference in their entirety) to cervical dystonia (for BoNT/B) (see for example, https://www.medicines.org.uk/emc/medicine/20568, herein incorporated by reference in its entirety).

In contrast to a cytotoxic protease (e.g. ricin, diphtheria toxin, pseudomonas exotoxin), which acts by killing its natural target cell, clostridial neurotoxins are non-cytotoxic proteases acting by transiently incapacitating the cellular function of its natural target cell. Importantly, a non-cytotoxic protease does not kill the natural target cell upon which it acts. In addition to clostridial neurotoxins (e.g. botulinum neurotoxin, marketed under names such as Dysport™, Neurobloc™, and Botox™), some of the best known examples of non-cytotoxic proteases include IgA proteases (see, for example, WO99/032272), and antarease proteases (see, for example, WO2011/022357).

The term “clostridial neurotoxin” as used herein means any polypeptide that enters a neuron and inhibits neurotransmitter release. This process encompasses the binding of the neurotoxin to a low or high affinity receptor, the internalisation of the neurotoxin, the translocation of the endopeptidase portion of the neurotoxin into the cytoplasm and the enzymatic modification of the neurotoxin substrate. More specifically, the term “neurotoxin” encompasses any polypeptide produced by Clostridium bacteria (clostridial neurotoxins) that enters a neuron and inhibits neurotransmitter release, and such polypeptides produced by recombinant technologies or chemical techniques. It is this di-chain form that is the active form of the toxin. The two chains are termed the heavy chain (H-chain), which has a molecular mass of approximately 100 kDa, and the light chain (L-chain), which has a molecular mass of approximately 50 kDa. Preferably, the clostridial neurotoxin is a botulinum neurotoxin (BoNT).

BoNT serotypes A to G can be distinguished based on inactivation by specific neutralising anti-sera, with such classification by serotype correlating with percentage sequence identity at the amino acid level. BoNT proteins of a given serotype are further divided into different subtypes on the basis of amino acid percentage sequence identity.

A clostridial neurotoxin of the invention may be selected from BoNT/A, BoNT/B, BoNT/C, BoNT/D, BoNT/E, BoNT/F, BoNT/G, BoNT/X, and TeNT (tetanus neurotoxin). Preferably, a clostridial neurotoxin is a botulinum neurotoxin, such as a botulinum neurotoxin selected from BoNT/A, BoNT/B, BoNT/C, BoNT/D, BoNT/E, BoNT/F, BoNT/G, and BoNT/X. For example, the clostridial neurotoxin may be selected from BoNT/A, BoNT/B, BoNT/C, BoNT/D, BoNT/E, BoNT/F, and BoNT/G.

In one embodiment the clostridial neurotoxin may be BoNT/A. An example of a BoNT/A neurotoxin amino acid sequence is provided as SEQ ID NO: 1 (UniProt accession number A5HZZ9). Another example of a BoNT/A neurotoxin amino acid sequence is provided as SEQ ID NO: 13 (UniProt accession number P10845). In another embodiment the clostridial neurotoxin may be BoNT/B. An example of a BoNT/B neurotoxin amino acid sequence is provided as SEQ ID NO: 2 (UniProt accession number B1 INP5). In another embodiment the clostridial neurotoxin may be BoNT/C. An example of a BoNT/C neurotoxin amino acid sequence is provided as SEQ ID NO: 3 (UniProt accession number P18640). In another embodiment the clostridial neurotoxin may be BoNT/D. An example of a BoNT/D neurotoxin amino acid sequence is provided as SEQ ID NO: 4 (UniProt accession number P19321). In another embodiment the clostridial neurotoxin may be BoNT/E. An example of a BoNT/E neurotoxin amino acid sequence is provided as SEQ ID NO: 5 (accession number WP_003372387). Another example of a BoNT/E neurotoxin amino acid sequence is provided as SEQ ID NO: 16 (UniProt accession number Q00496). In another embodiment the clostridial neurotoxin may be BoNT/F. An example of a BoNT/F neurotoxin amino acid sequence is provided as SEQ ID NO: 6 (UniProt accession number Q57236) or as SEQ ID NO: 9 (UniProt/UniParc accession number UPI0001 DE3DAC). In another embodiment the clostridial neurotoxin may be BoNT/G An example of a BoNT/G neurotoxin amino acid sequence is provided as SEQ ID NO: 7 (accession number WP_039635782). In another embodiment the clostridial neurotoxin may be BoNT/D-C. An example of a BoNT/D-C neurotoxin amino acid sequence is provided as SEQ ID NO: 8 (accession number BAM65681). In another embodiment the clostridial neurotoxin may be BoNT/X. An example of a BoNT/X neurotoxin amino acid sequence is provided as SEQ ID NO: 11 (accession number BAQ12790.1). In another embodiment the clostridial neurotoxin may be TeNT. An example of a TeNT neurotoxin amino acid sequence is provided as SEQ ID NO: 12 (UniProt accession number P04958).

In one embodiment, the clostridial neurotoxin consists of or comprises an amino acid sequence having at least 70%, preferably at least 75%, 80%, 85%, 90%, 95%, 99% or 100% sequence identity to any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 11. In one embodiment, the clostridial neurotoxin consists of or comprises an amino acid sequence of any of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, or SEQ ID NO: 11.

As used throughout the specification, the term “percent sequence identity” between two or more amino acid sequences means a function of the number of identical amino acids at identical positions shared by the aligned amino acid sequences. Thus, % identity as used herein may be calculated as the number of identical amino acids at each position in an alignment divided by the total number of amino acids in the aligned sequence, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, in particular a global alignment mathematical algorithm (such as described by Needleman and Wunsch, J. Mol. Biol. 48(3), 443-453, 1972), which will be familiar to a skilled person.

In one embodiment, the clostridial neurotoxin consists of or comprises an amino acid sequence having at least 70%, preferably at least 75%, 80%, 85%, 90%, 95%, 99% or 100% sequence identity to SEQ ID NO: 1. For example, the clostridial neurotoxin may consist of or comprise an amino acid sequence having at least 90% (more preferably at least 95%) sequence identity to SEQ ID NO: 1.

In one embodiment, the clostridial neurotoxin consists of or comprises an amino acid sequence of SEQ ID NO: 1 (e.g. BoNT/A).

In one embodiment, the clostridial neurotoxin consists of or comprises an amino acid sequence having at least 70%, preferably at least 75%, 80%, 85%, 90%, 95%, 99% or 100% sequence identity to SEQ ID NO: 13. For example, the clostridial neurotoxin may consist of or comprise an amino acid sequence having at least 90% (more preferably at least 95%) sequence identity to SEQ ID NO: 13. In one embodiment, the clostridial neurotoxin consists of or comprises an amino acid sequence of SEQ ID NO: 13 (e.g. BoNT/A).

The term “clostridial neurotoxin” is intended to embrace hybrid and chimeric clostridial neurotoxins. In one embodiment, the clostridial neurotoxin is a chimeric neurotoxin.

A hybrid clostridial neurotoxin comprises at least a portion of a light chain from one clostridial neurotoxin or subtype thereof, and at least a portion of a heavy chain from another clostridial neurotoxin or clostridial neurotoxin subtype. In one embodiment the hybrid clostridial neurotoxin may contain the entire light chain of a light chain from one clostridial neurotoxin subtype and the heavy chain from another clostridial neurotoxin subtype. In another embodiment, a chimeric clostridial neurotoxin may contain a portion (e.g. the binding domain) of the heavy chain of one clostridial neurotoxin subtype, with another portion of the heavy chain being from another clostridial neurotoxin subtype. Similarly or alternatively, the therapeutic element may comprise light chain portions from different clostridial neurotoxins. Such hybrid or chimeric clostridial neurotoxins are useful, for example, as a means of delivering the therapeutic benefits of such clostridial neurotoxins to subjects who are immunologically resistant to a given clostridial neurotoxin subtype, to subjects who may have a lower than average concentration of receptors to a given clostridial neurotoxin heavy chain binding domain, or to subjects who may have a protease-resistant variant of the membrane or vesicle toxin substrate (e.g., SNAP-25, VAMP and syntaxin). Hybrid and chimeric clostridial neurotoxins are described in U.S. Pat. No. 8,071,110, which publication is hereby incorporated by reference in its entirety. Thus, in one embodiment, the clostridial neurotoxin (or fragment thereof) of the invention is a hybrid clostridial neurotoxin, or a chimeric clostridial neurotoxin.

The term “chimeric neurotoxin” may be used herein means a neurotoxin comprising one or more domains originating from a first neurotoxin and one or more domains originating from a second neurotoxin. For example, a chimeric neurotoxin may comprise an LH_{N} domain originating from a first neurotoxin serotype or subtype and a H_{C} domain originating from a second neurotoxin serotype or subtype. Another example of a chimeric neurotoxin is a neurotoxin comprising an LH_{N} H_{CN} domain originating from a first neurotoxin serotype or subtype and a H_{CC} domain originating from a second neurotoxin serotype or subtype. A further example of a chimeric neurotoxin is a neurotoxin comprising an LH_{N} domain from a first neurotoxin serotype or subtype and an activation loop from a second neurotoxin serotype or subtype. Examples of chimeric neurotoxins are provided in WO2017191315 and WO2016156113, both herein incorporated by reference in their entirety.

The term “H_{C} domain” as used herein refers to a functionally distinct region of the neurotoxin heavy chain with a molecular weight of approximately 50 kDa that enables the binding of the neurotoxin to a receptor located on the surface of the target cell. The H_{C} domain consists of two structurally distinct subdomains, the “H_{CN} subdomain” (N-terminal part of the H_{C} domain) and the “H_{CC} subdomain” (C-terminal part of the H_{C} domain, also named H_{CC} domain), each of which having a molecular weight of approximately 25 kDa. A H_{CC} domain is capable of binding to a clostridial neurotoxin protein receptor.

The term “LH_{N} domain” as used herein refers to a neurotoxin region that is distinct from the H_{C} domain, and which consists of an endopeptidase domain (“L” or “light chain”) and of a domain responsible for translocation of the endopeptidase into the cytoplasm (H_{N} domain of the heavy chain). An endopeptidase domain (“L” or “light chain”) is capable of cleaving a SNARE protein.

Exemplary L, H_{N}, H_{CN} and H_{CC} domains are shown in Table 1.

TABLE 1
Exemplary L, H_{N}, H_{CN} and H_{CC} domains

| | Accession | SEQ ID | | | | |
|---|---|---|---|---|---|---|
| BoNT | Number | NO: | L | H_{N} | H_{CN} | H_{CC} |
| BoNT/A1 | A5HZZ9 | 1 | 1-448 | 449-872 | 873-1094 | 1095-1296 |
| BoNT/B1 | B1INP5 | 2 | 1-441 | 442-859 | 860-1081 | 1082-1291 |
| BoNT/C1 | P18640 | 3 | 1-449 | 450-867 | 868-1095 | 1096-1291 |
| BoNT/D | P19321 | 4 | 1-442 | 443-863 | 864-1082 | 1083-1276 |
| BoNT/E1 | WP_003372387 | 5 | 1-423 | 424-846 | 847-1069 | 1070-1252 |
| BoNT/F1 | Q57236 | 6 | 1-439 | 440-865 | 866-1087 | 1088-1278 |
| BoNT/F7 | UPI0001DE3DAC | 9 | 1-508 | 509-862 | 863-1076 | 1077-1268 |
| BoNT/G | WP_039635782 | 7 | 1-446 | 447-864 | 865-1089 | 1090-1297 |
| BoNT/DC | BAM65681 | 8 | 1-442 | 443-863 | 864-1091 | 1092-1285 |

| | | | | | |
|---|---|---|---|---|---|
| BoNT/X | BAQ12790.1 | 11 | 1-439 | 440-892 | 893-1306 |

The above-identified reference sequences should be considered a guide, as slight variations may occur according to sub-serotypes. By way of example, US 2007/0166332 (hereby incorporated by reference in its entirety) cites slightly different clostridial sequences.

The term “activation loop” refers to a polypeptide domain comprising a proteolytic cleavage site. Activation loops of neurotoxins have been described in the art, such as in WO2016156113 (hereby incorporated by reference in its entirety).

For example, the chimeric neurotoxin may comprise a LH_{N} domain from a first neurotoxin covalently linked to a H_{C} domain from a second neurotoxin, preferably wherein said first and second neurotoxins are different,

wherein the C-terminal amino acid residue of said LH_{N} domain corresponds to the first amino acid residue of the 3₁₀ helix separating the LH_{N} and H_{C} domains in said first neurotoxin, and
wherein the N-terminal amino acid residue of said H_{C} domain corresponds to the second amino acid residue of the 3₁₀ helix separating the LH_{N} and H_{C} domains in said second neurotoxin.

In one embodiment, the clostridial neurotoxin is a chimeric neurotoxin which comprises an H_{C} domain from a BoNT/B and an LH_{N} domain from a BoNT/A, BoNT/C, BoNT/D, BoNT/E, BoNT/F, or BoNT/G (e.g. BoNT/A).

For example, in one embodiment, the H_{C} domain consists of or comprises an amino acid sequence corresponding to amino acid residues 860 to 1291 of SEQ ID NO: 2 (e.g. BoNT/B), or an amino acid sequence having at least 70%, preferably at least 75%, 80%, 85%, 90%, 95% or 99% sequence identity thereto, and the LH_{N} domain consists of or comprises an amino acid sequence selected from the group consisting of:
amino acid residues 1 to 872 of SEQ ID NO: 1 (e.g. BoNT/A), or a sequence having at least 70%, preferably at least 75%, 80%, 85%, 90%, 95% or 99% sequence identity thereto,
amino acid residues 1 to 867 of SEQ ID NO: 3, or a sequence having at least 70%, preferably at least 75%, 80%, 85%, 90%, 95% or 99% sequence identity thereto,
amino acid residues 1 to 863 of SEQ ID NO: 4, or a sequence having at least 70%, preferably at least 75%, 80%, 85%, 90%, 95% or 99% sequence identity thereto,
amino acid residues 1 to 846 of SEQ ID NO: 5, or a sequence having at least 70%, preferably at least 75%, 80%, 85%, 90%, 95% or 99% sequence identity thereto,
amino acid residues 1 to 865 of SEQ ID NO: 6, or a sequence having at least 70%, preferably at least 75%, 80%, 85%, 90%, 95% or 99% sequence identity thereto,
amino acid residues 1 to 864 of SEQ ID NO: 7, or a sequence having at least 70%, preferably at least 75%, 80%, 85%, 90%, 95% or 99% sequence identity thereto,
amino acid residues 1 to 863 of SEQ ID NO: 8, or a sequence having at least 70%, preferably at least 75%, 80%, 85%, 90%, 95% or 99% sequence identity thereto, and
amino acid residues 1 to 862 of SEQ ID NO: 9, or a sequence having at least 70%, preferably at least 75%, 80%, 85%, 90%, 95% or 99% sequence identity thereto.

In one embodiment, a clostridial neurotoxin of the invention may be a chimeric clostridial neurotoxin comprising (preferably consisting of) a BoNT/A light-chain and translocation domain, and a BoNT/B receptor binding domain (H_{C} domain) or a portion thereof (e.g. BoNT/A LH_{N}-BoNT/B H_{C}). A suitable chimeric and/or hybrid clostridial neurotoxin may be one taught in WO 2017/191315 A1, which is incorporated herein by reference. A chimeric clostridial neurotoxin may comprise (preferably consist of) a sequence of SEQ ID NO: 10.

The BoNT/A LH_{N} domain may be covalently linked to the BoNT/B H_{C} domain. Said chimeric BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

Reference herein to the “first amino acid residue of the 3₁₀ helix separating the LH_{N} and H_{C} domains of BoNT/A” means the N-terminal residue of the 3₁₀ helix separating the LH_{N} and H_{C} domains. Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (=0.2 nm) along the helical axis, and has 10 atoms in the ring formed by making the hydrogen bond. Most importantly, the N—H group of an amino acid forms a hydrogen bond with the C═O group of the amino acid three residues earlier; this repeated i+3→i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publicly available crystal structures of botulinum neurotoxins, for example 3BTA (http://www.rcsb.org/pdb/explore/explore.do?structureId=3BTA) and 1 EPW (http://www.rcsb.org/pdb/explore/explore.do?structureId=1EPW) for botulinum neurotoxins A1 and B1 respectively.

In silico modelling and alignment tools which are publicly available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{CN}” junction is structurally highly conserved which renders it an ideal region to superimpose different serotypes.

For example, the following methodology may be used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3BTA.pdb structure;
4. The superposed pdb files were inspected to locate the 3₁₀ helix at the start of the H_{C} domain of BoNT/A1, and corresponding residues in the other serotype were then identified;
5. The other BoNT serotype sequences were aligned with Clustal Omega in order to check that corresponding residues were correct.

Examples of LH_{N}, H_{C} and 3₁₀ helix domains determined by this method are presented below:

| | Accession | | | |
|---|---|---|---|---|
| | Number (Plus | | | |
| | Sequence | | | |
| Neuro- | Version | | | |
| toxin | after Decimal) | LH_{N} | H_{C} | 3₁₀ helix |
| BoNT/A1 | A5HZZ9.1 | 1-872 | 873-1296 | ⁸⁷²NIINTS⁸⁷⁷ |
| (SEQ ID | | | | |
| NO: 1) | | | | |
| BoNT/A2 | X73423.3 | 1-872 | 873-1296 | ⁸⁷²NIVNTS⁸⁷⁷ |
| BoNT/A3 | DQ185900.1 | 1-872 | 873-1292 | ⁸⁷²NIVNTS⁸⁷⁷ |
| | (aka Q3LRX9.1) | | | |
| BoNT/A4 | EU341307.1 | 1-872 | 873-1296 | ⁸⁷²NITNAS⁸⁷⁷ |
| | (aka Q3LRX8.1) | | | |
| BoNT/A5 | EU679004.1 | 1-872 | 873-1296 | ⁸⁷²NIINTS⁸⁷⁷ |
| | (aka C1IPK2.1) | | | |
| BoNT/A6 | FJ981696.1 | 1-872 | 873-1296 | ⁸⁷²NIINTS⁸⁷⁷ |
| BoNT/A7 | JQ954969.1 | 1-872 | 873-1296 | ⁸⁷²NIINTS⁸⁷⁷ |
| | (aka K4LN57.1) | | | |
| BoNT/A8 | KM233166.1 | 1-872 | 873-1297 | ⁸⁷²NITNTS⁸⁷⁷ |
| BoNT/B1 | B1INP5.1 | 1-859 | 860-1291 | ⁸⁵⁹EILNNI⁸⁶⁴ |
| (a.k.a. | | | | |
| SEQ ID | | | | |
| NO: 2) | | | | |
| BoNT/B2 | AB084152.1 | 1-859 | 860-1291 | ⁸⁵⁹EILNNI⁸⁶⁴ |
| | (aka Q8GR96.1) | | | |
| BoNT/B3 | EF028400.1 | 1-859 | 860-1291 | ⁸⁵⁹EILNNI⁸⁶⁴ |
| | (aka A2I2S2.1) | | | |
| BoNT/B4 | EF051570.1 | 1-859 | 860-1291 | ⁸⁵⁹EILNNI⁸⁶⁴ |
| | (aka A2I2W0.1) | | | |
| BoNT/B5 | EF033130.1 | 1-859 | 860-1291 | ⁸⁵⁹DILNNI⁸⁶⁴ |
| | (aka A2I2U6.1) | | | |
| BoNT/B6 | AB302852.1 | 1-859 | 860-1291 | ⁸⁵⁹EILNNI⁸⁶⁴ |
| | (aka A8R089.1) | | | |
| BoNT/B7 | JQ354985.1 | 1-859 | 860-1291 | ⁸⁵⁹EILNNI⁸⁶⁴ |
| | (aka H9CNK9.1) | | | |
| BoNT/B8 | JQ964806.1 | 1-859 | 860-1292 | ⁸⁵⁹EILNNI⁸⁶⁴ |
| | (aka I6Z8G9.1) | | | |

Using structural analysis and sequence alignments, it was found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B,
wherein the C-terminal amino acid residue of the LH_{N} domain corresponds to the C-terminal amino acid residue of the α-helix located at the end (C-term) of LH_{N} domain of BoNT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera was to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity.

In a preferred embodiment, the clostridial neurotoxin is a chimeric neurotoxin which comprises an H_{C} domain from a BoNT/B and an LH_{N} domain from a BoNT/A.

The LH_{N} domain from BoNT/A may correspond to amino acid residues 1 to 872 of SEQ ID NO: 1, or a polypeptide sequence having at least 70% sequence identity thereto. The LH_{N} domain from BoNT/A may correspond to amino acid residues 1 to 872 of SEQ ID NO: 1, or a polypeptide sequence having at least 80%, 90% or 95% sequence identity thereto. Preferably, the LH_{N} domain from BoNT/A corresponds to amino acid residues 1 to 872 of SEQ ID NO: 1.

The H_{C} domain from BoNT/B may correspond to amino acid residues 860 to 1291 of SEQ ID NO: 2, or a polypeptide sequence having at least 70% sequence identity thereto. The H_{C} domain from BoNT/B may correspond to amino acid residues 860 to 1291 of SEQ ID NO: 2, or a polypeptide sequence having at least 80%, 90% or 95% sequence identity thereto. Preferably, the H_{C} domain from BoNT/B corresponds to amino acid residues 860 to 1291 of SEQ ID NO: 2. In a more preferred embodiment, the H_{C} domain consists of or comprises an amino acid sequence corresponding to amino acid residues 860 to 1291 of SEQ ID NO: 2, or an amino acid sequence having at least 70%, preferably at least 75%, 80%, 85%, 90%, 95% or 99% sequence identity thereto, and the LH_{N} domain comprises an amino acid sequence corresponding to amino acid residues 1 to 872 of SEQ ID NO: 1, or an amino acid sequence having at least 70%, preferably at least 75%, 80%, 85%, 90%, 95% or 99% sequence identity thereto.

The BoNT/AB chimera may comprise a BoNT/A LH_{N} domain and a BoNT/B H_{C} domain. The LH_{N} domain may correspond to amino acid residues 1 to 872 of BoNT/A (SEQ ID NO: 1) and the H_{C} domain may correspond to amino acid residues 860 to 1291 of BoNT/B (SEQ ID NO: 2).

A clostridial neurotoxin having one or more modifications (preferably in the amino acid sequence of the heavy chain) is referred to as a “modified clostridial neurotoxin” herein.

A BoNT/B H_{C} domain may further comprise at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

For example, in embodiments where the clostridial neurotoxin comprises an H_{C} domain from a BoNT/B (for example, where the clostridial neurotoxin is BoNT/B, or a chimeric neurotoxin which comprises an H_{C} domain from a BoNT/B), the clostridial neurotoxin may have one or more modifications in the amino acid sequence of the heavy chain (such as in the H_{C} domain) providing a “modified heavy chain”, preferably wherein said modified heavy chain binds to target nerve cells with a higher (or lower) affinity than the native neurotoxin. Such modifications in the H_{C} domain can include modifications of amino acid residues in the ganglioside binding site of the H_{CC} domain that can alter binding to the ganglioside of the target nerve cell, and/or modifications of amino acid residues in the protein receptor binding site of the H_{CC} domain that can alter binding to the protein receptor of the target nerve cell. Examples of such modified neurotoxins are described in WO2006027207 and WO2006114308, both of which are hereby incorporated by reference in their entirety.

In one embodiment of a modified clostridial neurotoxin according to the invention, the H_{CC} domain from a BoNT/B is modified as compared to the natural H_{CC} domain of said BoNT serotypes.

In a preferred embodiment, the H_{CC} domain from a BoNT/B neurotoxin comprises at least one amino acid residue mutation which increases the binding affinity of said H_{CC} domain for human Syt II as compared to the natural BoNT/B H_{CC} domain. Still, preferably, said at least one amino acid residue mutation increases the binding affinity of said H_{CC} domain for human Syt II by at least 50% as compared to the natural BoNT/B H_{CC} domain.

Such suitable amino acid residue mutations in the BoNT/B H_{CC} domain have been described in the art in WO2013180799 and WO2016154534, both herein incorporated by reference in their entirety.

In particular, said at least one amino acid residue mutation suitable for increasing the binding affinity of the BoNT/B H_{CC} domain for human Syt II by at least 50% as compared to the natural BoNT/B H_{CC} domain is an amino acid residue substitution, addition or deletion selected from the group consisting of: 1118M, 1183M, 1191M, 1191I, 1191Q, 1191T, 1199Y, 1199F, 1199L, 1201V, 1191C, 1191V, 1191L, 1191Y, 1199W, 1199E, 1199H, 1178Y, 1178Q, 1178A, 1178S, 1183C, 1183P and any combinations thereof. Preferably, said at least one amino acid residue mutation in the BoNT/B H_{CC} domain consists of two amino acid residue substitutions, additions or deletions selected from the group consisting of: 1191M and 1199L, 1191M and 1199Y, 1191M and 1199F, 1191Q and 1199L, 1191Q and 1199Y, 1191Q and 1199F, 1191M and 1199W, 1191M and 1178Q, 1191C and 1199W, 1191C and 1199Y, 1191C and 1178Q, 1191Q and 1199W, 1191V and 1199W, 1191V and 1199Y, or 1191V and 1178Q. Still preferably, said at least one amino acid residue mutation in the BoNT/B H_{CC} domain consists of the three amino acid residue substitutions, additions or deletions: 1191M, 1199W and 1178Q. More preferably, said at least one amino acid residue mutation in BoNT/B H_{CC} domain consists of the two amino acid residue substitutions, additions or deletions: 1191M and 1199Y.

In a more preferred embodiment, said at least one amino acid residue mutation suitable for increasing the binding affinity of the BoNT/B H_{CC} domain for human Syt II by at least 50% as compared to the natural BoNT/B H_{CC} domain is an amino acid residue substitution selected from the group consisting of: V1118M, Y1183M, E1191M, E1191I, E1191Q, E1191T, S1199Y, S1199F, S1199L, S1201V, E1191C, E1191V, E1191L, E1191Y, S1199W, S1199E, S1199H, W1178Y, W1178Q, W1178A, W1178S, Y1183C, Y1183P and any combinations thereof. Preferably, said at least one amino acid residue mutation in the BoNT/B H_{CC} domain consists of two amino acid residue substitutions selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q. Still preferably, said at least one amino acid residue mutation in the BoNT/B H_{CC} domain consists of the three amino acid residue substitutions: E1191M, S1199W and W1178Q. More preferably, said at least one amino acid residue mutation in BoNT/B H_{CC} domain consists of the two amino acid residue substitutions: E1191 M and S1199Y.

In other words, suitable amino acid residue substitution, addition or deletion in the BoNT/B H_{CC} subdomain include substitution mutations selected from the group consisting of: V1118M; Y1183M; E1191M; E1191I; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof. Suitable amino acid residue substitution, addition or deletion in the BoNT/B H_{CC} subdomain further include combinations of two substitution mutations selected from the group consisting of: E1191 M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q. Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191 M and S1199Y.

The modification may be a modification when compared to unmodified BoNT/B shown as SEQ ID NO: 2, wherein the amino acid residue numbering is determined by alignment with SEQ ID NO: 2. As the presence of a methionine residue at position 1 of SEQ ID NO: 2 is optional, the skilled person will take the presence/absence of the methionine residue into account when determining amino acid residue numbering. For example, where SEQ ID NO: 2 includes a methionine, the position numbering will be as defined above (e.g. E1191 will be E1191 of SEQ ID NO: 2). Alternatively, where the methionine is absent from SEQ ID NO: 2 the amino acid residue numbering should be modified by −1 (e.g. E1191 will be E1190 of SEQ ID NO: 2). Similar considerations apply when the methionine at position 1 of the other polypeptide sequences described herein is present/absent, and the skilled person will readily determine the correct amino acid residue numbering using techniques routine in the art.

In a preferred embodiment, the BoNT/B H_{CC} domain to be modified corresponds to amino acid residues 1082 to 1291 of SEQ ID NO: 2 (natural BoNT/B H_{CC} domain), or to an amino acid sequence having at least 70%, preferably at least 80%, 85%, 90%, 95% or 99% sequence identity thereto.

In one embodiment, the clostridial neurotoxin of the present invention can be both chimeric and modified, as described above. For example, in a preferred embodiment, the clostridial neurotoxin comprises (or consists of) the amino acid sequence SEQ ID NO: 10, or an amino acid sequence having at least 70%, preferably at least 75%, 80%, 85%, 90%, 95% or 99% sequence identity thereto.

In one embodiment, the clostridial neurotoxin of the present invention can be both chimeric and modified, as described above. For example, in a preferred embodiment, the clostridial neurotoxin comprises (or consists of) the amino acid sequence SEQ ID NO: 10 (e.g. BoNT/AB_{MY}), or an amino acid sequence having at least 70%, preferably at least 75%, 80%, 85%, 90%, 95% or 99% sequence identity thereto.

A modified clostridial neurotoxin may have one or more modifications in the amino acid sequence of the light chain, for example modifications in the substrate binding or catalytic domain which may alter or modify the SNARE protein specificity of the modified L-chain. Examples of such modified clostridial neurotoxins are described in WO 2010/120766 and US 2011/0318385, both of which are hereby incorporated by reference in their entirety.

A modified clostridial neurotoxin may comprise one or more modifications that increases or decreases the biological activity and/or the biological persistence of the modified clostridial neurotoxin. For example, a modified clostridial neurotoxin may comprise a leucine- or tyrosine-based motif, wherein said motif increases or decreases the biological activity and/or the biological persistence of the modified clostridial neurotoxin. Suitable leucine-based motifs include xDxxxLL, xExxxLL, xExxxlL, and xExxxLM (wherein x is any amino acid). Suitable tyrosine-based motifs include Y-x-x-Hy (wherein Hy is a hydrophobic amino acid). Examples of modified clostridial neurotoxins comprising leucine- and tyrosine-based motifs are described in WO 2002/08268, which is hereby incorporated by reference in its entirety.

As described below, a modified clostridial neurotoxin (or clostridial neurotoxin fragment) may be one that comprises one or more modifications that increases the isoelectric point of the clostridial neurotoxin when compared to an equivalent unmodified clostridial neurotoxin lacking said one or more modifications. Suitable modified clostridial neurotoxins are described above and in WO 2015/004461 A1 and WO 2016/110662 A1, which are incorporated herein by reference. Exemplary sequences include SEQ ID NOs: 61 and 42 described herein.

A modified clostridial neurotoxin may preferably comprise or consist of a sequence of SEQ ID NO: 15.

In one embodiment, a modified clostridial neurotoxin may preferably comprise or consist of a sequence of SEQ ID NO: 10.

In a particularly preferred embodiment, a clostridial neurotoxin of the invention may comprise a modified BoNT/A or fragment thereof (preferably a BoNT/A H_{C} domain or fragment thereof). The modified BoNT/A or fragment thereof may be one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such a modified BoNT/A or fragment thereof may demonstrate a reduction in, or absence of, side effects compared to the use of known BoNT/A. The increased tissue retention properties of the modified BoNT/A of the invention may also provide increased potency and/or duration of action and can allow for reduced dosages to be used compared to known clostridial toxin therapeutics (or increased dosages without any additional adverse effects), thus providing further advantages.

The modification may be a modification when compared to unmodified BoNT/A shown as SEQ ID NO: 1, wherein the amino acid residue numbering is determined by alignment with SEQ ID NO: 1. As the presence of a methionine residue at position 1 of SEQ ID NO: 1 (as well as the SEQ ID NOs corresponding to modified BoNT/A polypeptides or fragments thereof described herein) is optional, the skilled person will take the presence/absence of the methionine residue into account when determining amino acid residue numbering. For example, where SEQ ID NO: 1 includes a methionine, the position numbering will be as defined above (e.g. ASN 886 will be ASN 886 of SEQ ID NO: 1). Alternatively, where the methionine is absent from SEQ ID NO: 1 the amino acid residue numbering should be modified by −1 (e.g. ASN 886 will be ASN 885 of SEQ ID NO: 1). Similar considerations apply when the methionine at position 1 of the other polypeptide sequences described herein is present/absent, and the skilled person will readily determine the correct amino acid residue numbering using techniques routine in the art.

The amino acid residue(s) indicated for modification above are surface exposed amino acid residue(s).

A modified BoNT/A or fragment thereof may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277.

The term “one or more amino acid residue(s)” when used in the context of modified BoNT/A or fragment thereof preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A or fragment thereof may comprise 1-30, 3-20, or 5-10 amino acid modifications. More preferably, the term “one or more amino acid residue(s)” when used in the context of modified BoNT/A or fragment thereof means all of the indicated amino acid residue(s).

Preferably, beyond the one or more amino acid modification(s) at the indicated amino acid residue(s), the modified BoNT/A or fragment thereof does not contain any further amino acid modifications when compared to SEQ ID NO: 1.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A or fragment thereof that has an increased positive surface charge and increased isoelectric point when compared to the corresponding unmodified BoNT/A or fragment thereof.

The isoelectric point (pI) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group). Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pI). The isoelectric point is a standard concept in protein biochemistry with which the skilled person would be familiar.

The isoelectric point (pI) is therefore defined as the pH value at which a protein displays a net charge of zero. An increase in pI means that a higher pH value is required for the protein to display a net charge of zero. Thus, an increase in pI represents an increase in the net positive charge of a protein at a given pH. Conversely, a decrease in pI means that a lower pH value is required for the protein to display a net charge of zero. Thus, a decrease in pI represents a decrease in the net positive charge of a protein at a given pH.

Methods of determining the pI of a protein are known in the art and would be familiar to a skilled person. By way of example, the pI of a protein can be calculated from the average pKa values of each amino acid present in the protein (“calculated pI”). Such calculations can be performed using computer programs known in the art, such as the Compute pI/MW Tool from ExPASy (https://web.expasy.org/compute_pi/), which is the preferred method for calculating pI in accordance with the present invention. Comparisons of pI values between different molecules should be made using the same calculation technique/program.

Where appropriate, the calculated pI of a protein can be confirmed experimentally using the technique of isoelectric focusing (“observed pI”). This technique uses electrophoresis to separate proteins according to their pI. Isoelectric focusing is typically performed using a gel that has an immobilised pH gradient. When an electric field is applied, the protein migrates through the pH gradient until it reaches the pH at which it has zero net charge, this point being the pI of the protein. Results provided by isoelectric focusing are typically relatively low-resolution in nature, and thus the present inventors believe that results provided by calculated pI (as described above) are more appropriate to use.

Throughout the present specification, “pI” means “calculated pI” unless otherwise stated.

The pI of a protein may be increased or decreased by altering the number of basic and/or acidic groups displayed on its surface. This can be achieved by modifying one or more amino acids of the protein. For example, an increase in pI may be provided by reducing the number of acidic residues, or by increasing the number of basic residues.

A modified BoNT/A or fragment thereof of the invention may have a pI value that is at least 0.2, 0.4, 0.5 or 1 μl units higher than that of an unmodified BoNT/A (e.g. SEQ ID NO: 1) or fragment thereof. Preferably, a modified BoNT/A or fragment thereof may have a pI of at least 6.6, e.g. at least 6.8.

The properties of the 20 standard amino acids are indicated in the table below:

| Amino Acid | | | Side Chain |
|---|---|---|---|
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence or fragment thereof, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A or fragment thereof. In an amino acid substitution, an amino acid residue that forms part of the BoNT/A polypeptide sequence or fragment thereof is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Preferably, a modified BoNT/A or fragment thereof for use in the invention comprises between 4 and 40 amino acid modifications located in the clostridial toxin H_{CN} domain. Said modified BoNT/A or fragment thereof preferably also has pI of at least 6.6. Said modified BoNT/A preferably comprises modifications of at least 4 amino acids selected from: ASN 886, ASN 930, ASN 954, SER 955, GLN 991, ASN 1025, ASN 1026, and ASN 1052, wherein said modification comprises substitution of the amino acids with a lysine residue or an arginine residue. For example, said modified BoNT/A or fragment thereof may comprise modifications of at least 5 amino acids selected from: ASN 886, ASN 930, ASN 954, SER 955, GLN 991, ASN 1025, ASN 1026, ASN 1052, and GLN 1229, wherein said modification comprises substitution of the amino acids with a lysine residue or an arginine residue.

Methods for modifying proteins by substitution, insertion or deletion of amino acid residues are known in the art. By way of example, amino acid modifications may be introduced by modification of a DNA sequence encoding a polypeptide (e.g. encoding unmodified BoNT/A or a fragment thereof). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

Thus, a modified clostridial neurotoxin may have one or a number of such amino acid modifications (e.g. substitutions) when compared to wild-type BoNT/A, which increase the isoelectric point of the polypeptide. Without wishing to be bound by theory, it is believed that the increased net positive charge promotes electrostatic interactions between the polypeptide and anionic extracellular components, thereby promoting binding between the polypeptide and cell surface thus increasing retention at a site of administration and/or duration of action.

For modified BoNT/A polypeptides described above (e.g. SEQ ID NO: 15), one way in which these advantageous properties (which represent an increase in the therapeutic index) may be defined is in terms of the Safety Ratio of the modified BoNT/A. In this regard, undesired effects of a clostridial neurotoxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial neurotoxin can be assessed experimentally by Digital Abduction Score (DAS) assay, a measurement of muscle paralysis. The DAS assay may be performed by injection of 20 μl of clostridial neurotoxin, formulated in Gelatin Phosphate Buffer, into the mouse gastrocnemius/soleus complex, followed by assessment of Digital Abduction Score using the method of Aoki (Aoki KR, Toxicon 39: 1815-1820; 2001). In the DAS assay, mice are suspended briefly by the tail in order to elicit a characteristic startle response in which the mouse extends its hind limbs and abducts its hind digits. Following clostridial neurotoxin injection, the varying degrees of digit abduction are scored on a five-point scale (0=normal to 4=maximal reduction in digit abduction and leg extension).

The Safety Ratio of a clostridial neurotoxin may then be expressed as the ratio between the amount of toxin required for a 10% drop in a bodyweight (measured at peak effect within the first seven days after dosing in a mouse) and the amount of toxin required for a DAS score of 2. High Safety Ratio scores are therefore desired and indicate a toxin that is able to effectively paralyse a target muscle with little undesired off-target effects. A modified BoNT/A of the present invention may have a Safety Ratio that is higher than the Safety Ratio of an equivalent unmodified (native) botulinum toxin (e.g. SEQ ID NO: 1).

Thus, in one embodiment, a modified BoNT/A of the present invention has a Safety Ratio of at least 8 (for example, at least 8, 9, 10, 15, 20, 25, 30, 35, 40, 45 or 50), wherein Safety Ratio is calculated as: dose of toxin required for −10% bodyweight change (μg/mouse) divided by DAS ED₅₀ (μg/mouse) [ED₅₀=dose required to produce a DAS score of 2].

In one embodiment, a modified BoNT/A of the present invention has a Safety Ratio of at least 10. In one embodiment, a modified BoNT/A or fragment thereof of the present invention has a Safety Ratio of at least 15.

Clostridial neurotoxins comprising at least 70% sequence identity to SEQ ID NO: 15 are described in WO 2015/004461 A1, which is incorporated herein by reference in its entirety.

In one embodiment a clostridial neurotoxin comprising a polypeptide sequence having at least 70% sequence identity to SEQ ID NO: 15 and/or comprising a polypeptide sequence that is encoded by a nucleotide sequence having at least 70% sequence identity to SEQ ID NO: 14 comprises a substitution at one or more (preferably two or more, three or more, four or more, five or more or six or more, more preferably at all) of positions 930, 955, 991, 1026, 1052, 1229, and 886. The position numbering corresponds to the positions of SEQ ID NO: 1 and can be determined by aligning the polypeptide sequence with SEQ ID NO: 1 (unmodified/wild-type BoNT/A). As the presence of a methionine residue at position 1 of SEQ ID NO: 1 is optional, the skilled person will take the presence/absence of the methionine residue into account when determining amino acid residue numbering. For example, where SEQ ID NO: 1 includes a methionine, the position numbering will be as defined above (e.g. position 886 will be ASN 886 of SEQ ID NO: 1). Alternatively, where the methionine is absent from SEQ ID NO: 62 the amino acid residue numbering should be modified by −1 (e.g. position 886 will be ASN 885 of SEQ ID NO: 1). Similar considerations apply when the methionine at position 1 of the other polypeptide sequences described herein is present/absent, and the skilled person will readily determine the correct amino acid residue numbering using techniques routine in the art.

Preferably, the clostridial neurotoxin comprising a polypeptide sequence having at least 70% sequence identity to SEQ ID NO: 15 and/or comprising a polypeptide sequence that is encoded by a nucleotide sequence having at least 70% sequence identity to SEQ ID NO: 14 comprises lysine or arginine (more preferably lysine) at one or more of positions 930, 955, 991, 1026, 1052, 1229, and 886. In one embodiment, the clostridial neurotoxin comprises lysine or arginine (more preferably lysine) at least two, three, four, five, six or all of positions 930, 955, 991, 1026, 1052, 1229, and 886. Most preferably, the clostridial neurotoxin comprises lysine or arginine (more preferably lysine) at all of positions 930, 955, 991, 1026, 1052, 1229, and 886.

As described above, clostridial neurotoxins typically comprise targeting component (e.g. a C-terminal targeting component, H_{C} domain) mediating receptor binding at target cells, and clostridial neurotoxins may also have binding affinity for certain gangliosides that are present on the target cells. For example, BoNT/A, BoNT/D, BoNT/E, BoNT/F, and TeNT bind to synaptic vesicle protein 2 (SV2) with BoNT/A capable of binding to all three isoforms thereof (SV2A, SV2B, and SV2C) and BoNT/E capable of binding to the SV2A and SV2B isoforms. BoNT/B and BoNT/G bind to both isoforms (I and II) of synaptotagmin. Synaptotagmin and SV2 can be localized on synaptic vesicles and become exposed to extracellular space when the vesicles fuse with the presynaptic membrane. It is during this period that the clostridial neurotoxins bind to their protein receptors. For further information on suitable receptors/gangliosides, see Binz and Rummel (Journal of Neurochemistry, Volume 109, Issue 6, June 2009, Pages 1584-1595), incorporated herein by reference.

The clostridial neurotoxin of the invention may be referred to on the basis of the target receptor/ganglioside to which it binds. For example, the clostridial neurotoxin of the invention may bind a receptor selected from SV2 (SV2A, SV2B, and/or SV2C) and synaptotagmin (synaptotagmin I and/or II).

In a preferable embodiment, the clostridial neurotoxin binds SV2. In one embodiment, the clostridial neurotoxin binds SV2A, SV2B, and/or SV2C. Preferably, the clostridial neurotoxin may binds SV2A. In one embodiment, the clostridial neurotoxin binds SV2A, SV2B, and SV2C. The clostridial neurotoxin may comprise a BoNT H_{CC} domain selected from a BoNT/A H_{CC} domain (e.g. amino acids 1095-1296 of SEQ ID NO.: 1), a BoNT/D H_{CC} domain (e.g. amino acids 1083-1276 of SEQ ID NO.: 4), a BoNT/E H_{CC} domain (e.g. amino acids 1070-1252 of SEQ ID NO.: 5), and BoNT/F H_{CC} domain (e.g. amino acids 1088-1278 of SEQ ID NO.: 6 and/or amino acids 1077-1268 of SEQ ID NO.: 9).

In a preferred embodiment, the clostridial neurotoxin comprises a BoNT/A H_{CC} domain (e.g. amino acids 1095-1296 of SEQ ID NO.: 1).

In some embodiments, the clostridial neurotoxin binds Syt-I and/or Syt-II. The clostridial neurotoxin may comprise a BoNT/B H_{CC} domain (e.g. amino acids 1082-1291 of SEQ ID NO.: 2). In some embodiments, SV2A and/or SV2B (preferably SV2A and SV2B). The clostridial neurotoxin may comprise a BoNT/E H_{CC} domain (e.g. amino acids 1070-1252 of SEQ ID NO.: 5).

The clostridial neurotoxin of the present invention can be produced using recombinant technologies. Thus, in one embodiment, the clostridial neurotoxin of the invention is a recombinant clostridial neurotoxin.

In one embodiment, the clostridial neurotoxin is associated with BoNT complexing proteins, also known as non-toxic neurotoxin-associated proteins (NAP). In other words, the clostridial neurotoxin is administered to the human patient in association with, or combined with, BoNT complexing proteins. Hence, in one embodiment the clostridial neurotoxin is complexed with one or more BoNT complexing proteins.

In another embodiment, the clostridial neurotoxin is free of (or not associated with, or combined with) BoNT complexing proteins. In other words, the clostridial neurotoxin is administered to the human patient without being associated with, or combined with, BoNT complexing proteins.

Preferably, the clostridial neurotoxin (e.g. for use as described herein) is part of a pharmaceutical composition together with at least one pharmaceutically acceptable carrier. By “pharmaceutically acceptable carrier”, it is meant herein any component that is compatible with the other ingredients of the pharmaceutical composition, in particular with the clostridial neurotoxin, and which is not deleterious to the human patient. The pharmaceutically acceptable carrier can be selected on the basis of the desired route of administration, in accordance with standard pharmaceutical practices, and include, without limitation, excipients, diluents, adjuvants, propellants and salts.

Accordingly, the present invention further relates to a pharmaceutical composition for use in the treatment of a skin condition in a human patient, wherein said composition comprises the clostridial neurotoxin of the invention and at least one pharmaceutically acceptable carrier, and the dose of the clostridial neurotoxin to be administered to the patient is as described above. Also encompassed are corresponding uses and methods of treating a skin condition comprising administering a pharmaceutical composition of the invention to a human patient.

The clostridial neurotoxin of the present invention may preferably be formulated for intradermal administration.

A preferred route of administration is via intradermal administration. Preferably, intradermal administration means intradermal injection.

Preferably, said BoNT/A treating the same skin condition is a purified BoNT/A. As used herein, the term “purified BoNT/A” means a botulinum neurotoxin type A purified from a clostridial strain which naturally produces it (naturally-occurring clostridial strain). The purified BoNT/A may be associated with complexing proteins or free of complexing proteins. Examples of commercially available purified BoNT/A include Botox®, Dysport® and Xeomin®.

The doses of clostridial neurotoxin are herein preferably measured in nanograms.

Doses of clostridial neurotoxin according to the invention are to be understood as doses of active di-chain clostridial neurotoxin, i.e. without including the quantity of complexing proteins to which the neurotoxin may be associated with. In other words, it refers to the doses of active di-chain clostridial neurotoxin, whether said neurotoxin is administered to the patient in association to, or without, complexing proteins. As well-known to the skilled practitioner, an active di-chain clostridial neurotoxin is capable of binding to a membrane (e.g. cell membrane) receptor, translocating the light chain into the cytoplasm and of cleaving a SNARE protein, while complexing proteins do not display such biological activity (i.e. are not “active”).

Alternatively, the doses of clostridial neurotoxin may be measured in “Units” (U) of clostridial neurotoxin.

Indeed, as well known to the skilled practitioner, the potency of a clostridial neurotoxin is related to the quantity (e.g. nanograms) of neurotoxin required to achieve an LD50 (lethal dose 50) unit; one LD50 unit being defined as the median lethal intraperitoneal dose (as measured in mice). However, BoNT pharmaceutical preparations currently on the market contain different amount of 150 kD neurotoxin, but also of LD50 Units. Besides, in these preparations, the neurotoxin may, or may not, be associated with (i.e. combined with) non-toxic neurotoxin-associated proteins (NAP), also known as complexing proteins. For ease of conversion (as reported in Field et. al, “AbobotulinumtoxinA (Dysport®), OnabotulinumtoxinA (Botox®), and IncobotulinumtoxinA (Xeomin®) Neurotoxin Content and Potential Implications for Duration of Response in Patients”. Toxins 2018, 10(12), 535):
100 Units of Botox® (also known as OnabotulinumtoxinA) contains about 0.9 ng of 150 kD BoNT/A, as well as complexing proteins;
500 Units of Dysport® (also known as AbobotulinumtoxinA) contains about 2.69 ng of 150 kD BoNT/A, as well as complexing proteins;
100 Units of Xeomin® (also known as IncobotulinumtoxinA) contains about 0.40 ng of 150 kD BoNT/A, with no complexing proteins.

It should be noted that conversion values may vary slightly. For example, conversion values reported in Frevert, 2012 (“Content of botulinum neurotoxin in Botox®/Vistabel®, Dysport®/Azzalure®, and Xeomin®/Bocouture®”; Drugs R D. 2010; 10(2):67-73) are as follows:
100 Units of Botox® (also known as OnabotulinumtoxinA) contains about 0.73 ng of 150 kD BoNT/A, as well as complexing proteins;
100 Units of Dysport® (also known as AbobotulinumtoxinA) contains about 0.65 ng of 150 kD BoNT/A, as well as complexing proteins;
100 Units of Xeomin® (also known as IncobotulinumtoxinA) contains about 0.44 ng of 150 kD BoNT/A, with no complexing proteins;
100 Units of Neurobloc/Myobloc® (also known as RimabotulinumtoxinB) contains about 0.2 ng to about 1 ng of 150 kD BoNT/B, as well as complexing proteins.

The quantity of clostridial neurotoxin can be measured by the skilled practitioner according to methods conventionally used in the art to quantify proteins preferably at nanograms levels, including, among others, mass spectroscopy such as isotopic dilution mass spectroscopy (Muhoz et al., Quantification of protein calibrants by amino acid analysis using isotope dilution mass spectrometry, Anal. Biochem. 2011, 408, 124-131), or fluorimetric assay (Poras et al., Detection and Quantification of Botulinum Neurotoxin Type A by a Novel Rapid In Vitro Fluorimetric Assay, Appl Environ Microbiol. 2009 July; 75(13): 4382-4390).

Thus, the dosage ranges for administration of the clostridial neurotoxins of the present invention are those to produce the desired therapeutic and/or prophylactic effect. It will be appreciated that the dosage range required depends on the precise nature of the clostridial neurotoxin or composition, the route of administration, the nature of the formulation, the age of the subject, the nature, extent or severity of the subject's condition, contraindications, if any, and the judgement of the attending physician. Variations in these dosage levels can be adjusted using standard empirical routines for optimisation.

In one embodiment a dosage of the clostridial neurotoxin is a flat dose. A flat dose may be in the range of 50 μg to 250 μg, preferably 100 μg to 100 μg. In one embodiment a flat dose may be at least 50 μg, 100 μg, 500 μg, 1 ng, 50 ng, 100 ng, 500 ng, 1 μg or 50 μg. Said dose may be a single flat dose.

Fluid dosage forms are typically prepared utilising the clostridial neurotoxin and a pyrogen-free sterile vehicle. The clostridial neurotoxin, depending on the vehicle and concentration used, can be either dissolved or suspended in the vehicle. In preparing solutions the clostridial neurotoxin can be dissolved in the vehicle, the solution being made isotonic if necessary by addition of sodium chloride and sterilised by filtration through a sterile filter using aseptic techniques before filling into suitable sterile vials or ampoules and sealing. Alternatively, if solution stability is adequate, the solution in its sealed containers may be sterilised by autoclaving. Advantageously additives such as buffering, solubilising, stabilising, preservative or bactericidal, suspending or emulsifying agents and or local anaesthetic agents may be dissolved in the vehicle.

Dry powders, which are dissolved or suspended in a suitable vehicle prior to use, may be prepared by filling pre-sterilised ingredients into a sterile container using aseptic technique in a sterile area. Alternatively the ingredients may be dissolved into suitable containers using aseptic technique in a sterile area. The product is then freeze dried and the containers are sealed aseptically.

Parenteral suspensions, suitable for an administration route described herein, are prepared in substantially the same manner, except that the sterile components are suspended in the sterile vehicle, instead of being dissolved and sterilisation cannot be accomplished by filtration. The components may be isolated in a sterile state or alternatively it may be sterilised after isolation, e.g. by gamma irradiation.

Advantageously, a suspending agent for example polyvinylpyrrolidone is included in the composition(s) to facilitate uniform distribution of the components.

Administration in accordance with the present invention may take advantage of a variety of delivery technologies including microparticle encapsulation, or high-pressure aerosol impingement.

Intradermal administration may comprise intradermal injection with a needle such as a 30 gauge needle, preferably wherein the needle (such as a 30 gauge needle) is inserted into dermis of the skin at an angle of about 5°-15° relative to a surface (e.g. planar surface) of the skin. Preferably, the depth of the injection (depth relative to the surface of the skin) is around 0.2-0.3 (preferably around 0.25) inches. The term “planar surface” may refer to the contact area of the skin.

Where a range of values is herein provided, it shall be understood that, unless the context clearly dictates otherwise, each intervening value to the tenth of the unit between the upper and lower limits of that range is also specifically disclosed. Each smaller range between any stated value or intervening value in a stated range and any other stated or intervening value in that stated range is encompassed within this disclosure. It shall be further understood that any range of numerical values denoted herein by the expression “from a to b” means the range of numerical values extending from a to b (i.e. including the strict end points a and b).

Besides, the term “about” shall be understood herein as plus or minus (±) 5%, preferably ±4%, ±3%, ±2%, ±1%, ±0.5%, ±0.1%, of the numerical value of the number with which it is being used.

In a preferred embodiment, the dose of the clostridial neurotoxin of the invention to be administered for treating a skin condition in a human patient (i.e. therapeutic or cosmetic dose) is ranging from about 0.00025 ng to about 3 ng.

In a preferred embodiment, the therapeutic (or cosmetic) dose of the clostridial neurotoxin is ranging from about 0.0003 ng to about 2 ng, preferably from about 0.0004 ng to about 1.5 ng, from about 0.0005 ng to about 1 ng, still preferably from about 0.0006 ng to about 0.5 ng of said clostridial neurotoxin.

For example, the dose of the clostridial neurotoxin comprising a BoNT/A is preferably ranging from about 0.001 ng to about 2 ng.

It will nevertheless be appreciated that the dose range required depends on the precise nature of the clostridial neurotoxin, the maximum tolerated dose in the particular patient/subject (e.g. human patient/subject), the skin condition, the route of administration, the nature of the formulation, the age of the patient, the nature, extent or severity of the patient's condition, contraindications, if any, and the judgement of the attending physician. Variations in these dosage levels can be adjusted using standard empirical routines for optimisation.

Where are reference standard is referred to herein for the purpose of comparing a level of sebum (post-administration) relative to the reference standard—the reference standard may correspond to a level of sebum on the skin (e.g. epidermal layer) of a patient that has not been administered the clostridial neurotoxin. Additionally or alternatively, the reference standard may correspond to a level of sebum on the skin (e.g. epidermal layer) of the patient (e.g. the patient to which the clostridial neurotoxin has been administered) pre-administration of the clostridial neurotoxin.

Embodiments related to the various therapeutic uses of the invention are intended to be applied equally to methods of treatment, clostridial neurotoxins of the invention, and vice versa.

### CLAUSES

1. A method for treating a skin condition, said method comprising administering intradermally to a patient a clostridial neurotoxin, wherein following administration the clostridial neurotoxin induces:
secretion of one or more sebaceous lipid(s) selected from a squalene, a fatty acid, a cholesterol, and a wax ester to an epidermal layer of skin.

2. A clostridial neurotoxin for use in a method of treating a skin condition, said method comprising administering intradermally to a patient a clostridial neurotoxin, wherein following administration the clostridial neurotoxin induces:
secretion of one or more sebaceous lipid(s) selected from a squalene, a fatty acid, a cholesterol, and a wax ester to an epidermal layer of skin.

3. Non-therapeutic use of a clostridial neurotoxin for cosmetic treatment of the skin, wherein the clostridial neurotoxin induces:
secretion of one or more sebaceous lipid(s) selected from a squalene, a fatty acid, a cholesterol, and a wax ester to an epidermal layer of skin.

4. The non-therapeutic use according to clause 3, wherein the clostridial neurotoxin is administered by intradermal administration.

5. The method according to clause 1 or the clostridial neurotoxin for use according to clause 2, wherein the skin condition is a condition associated with an aberrant sebaceous lipid level, wherein the sebaceous lipid is present on an epidermal layer at a lower level than on an epidermal layer of a patient that does not have the skin condition.

6. The method according to clause 1 or clause 5, or the clostridial neurotoxin for use according to clause 2 or clause 5, wherein the skin condition is one or more condition selected from acne, atopic dermatitis, netherton syndrome, psoriasis, dehydrated skin (e.g. dry or cracked skin), actinic keratosis, rosacea, carbuncle, eczema, cellulitis, dermatitis, skin cancer and keratosis pilaris.

7. The method according to any one of clauses 1, 5 or 6, or the clostridial neurotoxin for use according to any one of clauses 2, 5 or 6, wherein the skin condition is one or more condition selected from psoriasis, eczema, and dermatitis.

8. The method, the clostridial neurotoxin for use or non-therapeutic use according to any one of the preceding clause, wherein following administration of the clostridial neurotoxin:
no increase in the level of sebum on the skin of the patient is induced relative to a reference standard; and preferably no decrease in the level of sebum on the skin of the patient is induced relative to a reference standard;
wherein the reference standard corresponds to a level of sebum on the skin of a subject that has not been administered the clostridial neurotoxin.

9. The method, the clostridial neurotoxin for use or non-therapeutic use of any one of the preceding clauses, wherein the patient does not have oily skin prior to and/or subsequent to administration of the clostridial neurotoxin.

10. The method, the clostridial neurotoxin for use or non-therapeutic use of any one of the preceding clauses, wherein said clostridial neurotoxin is a BoNT/A neurotoxin.

11. The method, the clostridial neurotoxin for use or non-therapeutic use according to any one of the preceding clauses, wherein said clostridial neurotoxin is a chimeric neurotoxin.

12. The method, the clostridial neurotoxin for use or non-therapeutic use according to clause 11, wherein the chimeric neurotoxin is selected from the group consisting of BoNT/DC and BoNT/X.

13. The method, the clostridial neurotoxin for use or non-therapeutic use according to clause 11, wherein the chimeric neurotoxin comprises a LH_{N} domain from a first neurotoxin covalently linked to a H_{C} domain from a second neurotoxin, preferably
wherein said first and second neurotoxins are different,
wherein the C-terminal amino acid residue of said LH_{N} domain corresponds to the first amino acid residue of the 3₁₀ helix separating the LH_{N} and H_{C} domains in said first neurotoxin, and
wherein the N-terminal amino acid residue of said H_{C} domain corresponds to the second amino acid residue of the 3₁₀ helix separating the LH_{N} and H_{C} domains in said second neurotoxin.

14. The method, the clostridial neurotoxin for use or non-therapeutic use according to clause 13, wherein said first neurotoxin is BoNT/A and wherein said second neurotoxin is BoNT/B.

15. The method, the clostridial neurotoxin for use or non-therapeutic use according to any one of the preceding clauses, wherein the intradermal administration comprises administering a dose of the clostridial neurotoxin ranging from about 0.00025 ng-3 ng to the site of administration.

16. The method, the clostridial neurotoxin for use or non-therapeutic use according to any one of the preceding clauses, wherein the intradermal administration comprises intradermal injection with a 30 gauge needle, preferably wherein the 30 gauge needle is inserted into dermis of the skin at an angle of about 5°-15° relative to a planar surface of the skin.

17. The method, the clostridial neurotoxin for use or non-therapeutic use according to any one of the preceding clauses, wherein the epidermal layer is one or more selected from the Stratum basale, the Stratum spinosum, the Stratum granulosum, or the Stratum corneum.

18. The method, the clostridial neurotoxin for use or non-therapeutic use according to any one of the preceding clauses, wherein the epidermal layer is the Stratum corneum.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position—Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

A R N D C Q E G H I L K M F P S T W Y V
A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5
F −2 −3 −3 −3 −2 −3 −3 −3 -1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

### Conservative Amino Acid Substitutions

Basic: arginine, lysine, histidine
Acidic: glutamic acid, aspartic acid
Polar: glutamine, asparagine
Hydrophobic: leucine, isoleucine, valine
Aromatic: phenylalanine, tryptophan, tyrosine
Small: glycine, alanine, serine, threonine, methionine

In addition to the 20 standard amino acids, non-standard amino acids (such as 4-hydroxyproline, 6-N-methyl lysine, 2-aminoisobutyric acid, isovaline and a-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

It must be noted that as used herein and in the appended claims, the singular forms “a”, “an”, and “the” include plural referents unless the context clearly dictates otherwise. Thus, for example, reference to “a clostridial neurotoxin” includes a plurality of such clostridial neurotoxins and reference to “the clostridial neurotoxin” includes reference to one or more clostridial neurotoxin and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

The invention will now be described, by way of example only, with reference to the following Figures and Examples.

### BRIEF DESCRIPTION OF THE DRAWINGS

Embodiments of the invention will now be described, by way of example only, with reference to the following Figures and Examples.

In all Figures, Gp1=Group 1; Gp2=Group 2; Gp3=Group 3; Gp4=Group 4; Gp5=Group 5; Dysport=BoNT/A; U=Unit; retention time=gas chromatography retention time.

FIG. 1 shows (a) an image of a Rhino mouse which has been administered with BoNT/A (Dysport) via intradermal injection in the back (the injection spots are the four darkened spots in the middle of the back which appear to form four corners of a square). Levels of sebum subsequently measured on the back skin of the Rhino mouse after injection (or application of control substance adapalene) are shown in (B). Dysport=BoNT/A.

FIG. 2 shows levels of a representative fatty acid in the sebum collected from the back skin of Rhino mice in the different groups, 15 days after injection. The retention time for this fatty acid was 22.1 minutes (this fatty acid corresponds to the peak labelled “FA4” in FIG. 17B). The data presents the average of six samples, together with the standard deviation.

FIG. 3 shows levels of squalene in the sebum collected from the back skin of Rhino mice in the different groups, 15 days after injection. The retention time for squalene was 25 minutes (squalene corresponds to the peak labelled “ST1” in FIG. 17B). The data presents the average of six samples, together with the standard deviation.

FIG. 4 shows levels of cholesterol in the sebum collected from the back skin of Rhino mice in the different groups, 15 days after injection. The retention time for cholesterol was 24.4 minutes (cholesterol corresponds to the peak labelled “Chol” in FIG. 17B). The data presents the average of six samples, together with the standard deviation.

FIG. 5 (a)-(c) shows levels of three different wax esters in the sebum collected from the back skin of Rhino mice in the different groups, 15 days after injection. The retention time for “wax ester 2” shown in FIG. 5A was 31 min; the retention time for “wax ester 3” shown in FIG. 5B was 35.5 min; the retention time for “wax ester 4” shown in FIG. 5C was 38 minutes. Wax esters 2-3 correspond to the peaks labelled WX2-3 (respectively) in FIG. 17C. The data presents the average of six samples, together with the standard deviation.

FIG. 6 shows levels of cholesteryl ester in the sebum collected from the back skin of Rhino mice in the different groups, 15 days after injection. The retention time for this cholesteryl ester was 41 mins (this cholesteryl ester corresponds to the peak labelled “CE” in FIG. 17D). The data presents the average of six samples, together with the standard deviation.

FIG. 7 shows levels of Substance P (μg/g tissue) in back skin samples collected from the back skin of Rhino mice in the different groups, 15 days after injection. ***p<0.001 versus vehicle (Gp1), One-Way ANOVA followed by Dunnet posttest.

FIG. 8 shows evolution of back skin (a) erythema and (b) scaling in Rhino mice in the different groups. Scoring was performed on days 1, 5, 10 and 15.

FIG. 9 shows the size of sebaceous glands surface in back skin of Rhino mice in the different groups, 15 days after injection. ***p<0.001 versus vehicle (Gp1), One-Way ANOVA followed by Dunnet posttest.

FIG. 10 shows the size of utriculi surface in back skin of Rhino mice in the different groups, 15 days after injection. ***p<0.001 versus vehicle (Gp1), One-Way ANOVA followed by Dunnet posttest.

FIG. 11 shows back skin epidermis thickness in back skin of Rhino mice in the different groups, 15 days after injection. *p<0.05, ***p<0.001 versus vehicle (Gp1), One-Way ANOVA followed by Dunnet posttest.

FIG. 12 shows levels of dermis inflammation in back skin of Rhino mice in the different groups, 15 days after injection. *p<0.05, **p<0.01 versus vehicle (Gp1), One-Way ANOVA followed by Dunnet posttest.

FIG. 13 shows levels of keratinocyte proliferation in epidermis of the back of Rhino mice in the different groups, 15 days after injection. **p<0.01 versus vehicle (Gp1), One-Way ANOVA followed by Dunnet posttest.

FIG. 14 shows levels of fibroblast proliferation in dermis of the back of Rhino mice in the different groups, 15 days after injection. *p<0.05 versus vehicle (Gp1), One-Way ANOVA followed by Dunnet posttest.

FIG. 15 shows levels of sebocyte proliferation in dermis of the back of Rhino mice in the different groups, 15 days after injection.

FIG. 16 shows levels of IL-1 alpha (ng/g tissue) in back skin samples collected from the back skin of Rhino mice in the different groups, 15 days after injection. ***p<0.001 versus vehicle (Gp1), One-Way ANOVA followed by Dunnet posttest.

FIG. 17 shows exemplary chromatograms generated during the lipidomics analysis described herein for each of groups 1-5. Expanded views of this chromatogram are show in FIG. 17B-D. A=full mean chromatogram obtained per group in sebum samples (e.g. lipid extract of sebum sample, comprising the sebaceous lipids); B=expanded view showing peaks corresponding to fatty acid (peak labelled FA4), and squalene (peak labelled ST1), and cholesterol (peak labelled Chol) obtained per group in sebum samples; C=expanded view showing peaks corresponding to waxes/wax esters (peaks labelled WX1, WX2, WX3 and WX4), obtained per group in sebum samples; D=expanded view showing peaks corresponding to cholesteryl esters (peak labelled CE), obtained per group in sebum samples.

### SEQUENCE LISTING

Where an initial Met amino acid residue or a corresponding initial codon is indicated in any of the following SEQ ID NOs, said residue/codon is optional.

SEQ ID NO: 1-BONT/A1, accession number A5HZZ9, amino acid sequence
MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIY
STDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESLE
VDTNPLLGAGKFATDPAVTLAHELIHAGHRLYGIAINPNRVFKVNTNAYY
EMSGLEVSFEELRTFGGHDAKFIDSLQENEFRLYYYNKFKDIASTLNKAK
SIVGTTASLQYMKNVFKEKYLLSEDTSGKFSVDKLKFDKLYKMLTEIYTE
DNFVKFFKVLNRKTYLNFDKAVFKINIVPKVNYTIYDGFNLRNTNLAANFN
GQNTEINNMNFTKLKNFTGLFEFYKLLCVRGIITSKTKSLDKGYNKALND
LCIKVNNWDLFFSPSEDNFTNDLNKGEEITSDTNIEAAEENISLDLIQQYY
LTFNFDNEPENISIENLSSDIIGQLELMPNIERFPNGKKYELDKYTMFHYL
RAQEFEHGKSRIALTNSVNEALLNPSRVYTFFSSDYVKKVNKATEAAMF
LGWVEQLVYDFTDETSEVSTTDKIADITIIIPYIGPALNIGNMLYKDDFVGA
LIFSGAVILLEFIPEIAIPVLGTFALVSYIANKVLTVQTIDNALSKRNEKWDE
VYKYIVTNWLAKVNTQIDLIRKKMKEALENQAEATKAIINYQYNQYTEEEK
NNINFNIDDLSSKLNESINKAMININKFLNQCSVSYLMNSMIPYGVKRLED
FDASLKDALLKYIYDNRGTLIGQVDRLKDKVNNTLSTDIPFQLSKYVDNQ
RLLSTFTEYIKNIINTSILNLRYESNHLIDLSRYASKINIGSKVNFDPIDKNQI
QLFNLESSKIEVILKNAIVYNSMYENFSTSFWIRIPKYFNSISLNNEYTIINC
MENNSGWKVSLNYGEIIWTLQDTQEIKQRVVFKYSQMINISDYINRWIFV
TITNNRLNNSKIYINGRLIDQKPISNLGNIHASNNIMFKLDGCRDTHRYIWI
KYFNLFDKELNEKEIKDLYDNQSNSGILKDFWGDYLQYDKPYYMLNLYD
PNKYVDVNNVGIRGYMYLKGPRGSVMTTNIYLNSSLYRGTKFIIKKYASG
NKDNIVRNNDRVYINVVVKNKEYRLATNASQAGVEKILSALEIPDVGNLS
QVVVMKSKNDQGITNKCKMNLQDNNGNDIGFIGFHQFNNIAKLVASNW
YNRQIERSSRTLGCSWEFIPVDDGWGERPL
SEQ ID NO: 2-BONT/B1, accession number B1INP5, amino acid sequence
MPVTINNFNYNDPIDNNNIIMMEPPFARGTGRYYKAFKITDRIWIIPERYT
FGYKPEDFNKSSGIFNRDVCEYYDPDYLNTNDKKNIFLQTMIKLFNRIKS
KPLGEKLLEMIINGIPYLGDRRVPLEEFNTNIASVTVNKLISNPGEVERKK
GIFANLIIFGPGPVLNENETIDIGIQNHFASREGFGGIMQMKFCPEYVSVF
NNVQENKGASIFNRRGYFSDPALILMHELIHVLHGLYGIKVDDLPIVPNEK
KFFMQSTDAIQAEELYTFGGQDPSIITPSTDKSIYDKVLQNFRGIVDRINK
VLVCISDPNININIYKNKFKDKYKFVEDSEGKYSIDVESFDKLYKSLMFGF
TETNIAENYKIKTRASYFSDSLPPVKIKNLLDNEIYTIEEGFNISDKDMEKE
YRGQNKAINKQAYEEISKEHLAVYKIQMCKSVKAPGICIDVDNEDLFFIAD
KNSFSDDLSKNERIEYNTQSNYIENDFPINELILDTDLISKIELPSENTESLT
DFNVDVPVYEKQPAIKKIFTDENTIFQYLYSQTFPLDIRDISLTSSFDDALL
FSNKVYSFFSMDYIKTANKVVEAGLFAGWVKQIVNDFVIEANKSNTMDKI
ADISLIVPYIGLALNVGNETAKGNFENAFEIAGASILLEFIPELLIPVVGAFLL
ESYIDNKNKIIKTIDNALTKRNEKWSDMYGLIVAQWLSTVNTQFYTIKEGM
YKALNYQAQALEEIIKYRYNIYSEKEKSNINIDFNDINSKLNEGINQAIDNIN
NFINGCSVSYLMKKMIPLAVEKLLDFDNTLKKNLLNYIDENKLYLIGSAEY
EKSKVNKYLKTIMPFDLSIYTNDTILIEMFNKYNSEILNNIILNLRYKDNNLI
DLSGYGAKVEVYDGVELNDKNQFKLTSSANSKIRVTQNQNIIFNSVFLDF
SVSFWIRIPKYKNDGIQNYIHNEYTIINCMKNNSGWKISIRGNRIIWTLIDIN
GKTKSVFFEYNIREDISEYINRWFFVTITNNLNNAKIYINGKLESNTDIKDIR
EVIANGEIIFKLDGDIDRTQFIWMKYFSIFNTELSQSNIEERYKIQSYSEYL
KDFWGNPLMYNKEYYMFNAGNKNSYIKLKKDSPVGEILTRSKYNQNSK
YINYRDLYIGEKFIIRRKSNSQSINDDIVRKEDYIYLDFFNLNQEWRVYTYK
YFKKEEEKLFLAPISDSDEFYNTIQIKEYDEQPTYSCQLLFKKDEESTDEI
GLIGIHRFYESGIVFEEYKDYFCISKWYLKEVKRKPYNLKLGCNWQFIPK
DEGWTE
SEQ ID NO: 3-BoNT/C1, accession number P18640, amino acid sequence
MPITINNFNYSDPVDNKNILYLDTHLNTLANEPEKAFRITGNIWVIPDRFS
RNSNPNLNKPPRVTSPKSGYYDPNYLSTDSDKDPFLKEIIKLFKRINSREI
GEELIYRLSTDIPFPGNNNTPINTFDFDVDFNSVDVKTRQGNNWVKTGSI
NPSVIITGPRENIIDPETSTFKLTNNTFAAQEGFGALSIISISPRFMLTYSNA
TNDVGEGRFSKSEFCMDPILILMHELNHAMHNLYGIAIPNDQTISSVTSNI
FYSQYNVKLEYAEIYAFGGPTIDLIPKSARKYFEEKALDYYRSIAKRLNSIT
TANPSSFNKYIGEYKQKLIRKYRFVVESSGEVTVNRNKFVELYNELTQIF
TEFNYAKIYNVQNRKIYLSNVYTPVTANILDDNVYDIQNGFNIPKSNLNVL
FMGQNLSRNPALRKVNPENMLYLFTKFCHKAIDGRSLYNKTLDCRELLV
KNTDLPFIGDISDVKTDIFLRKDINEETEVIYYPDNVSVDQVILSKNTSEHG
QLDLLYPSIDSESEILPGENQVFYDNRTQNVDYLNSYYYLESQKLSDNVE
DFTFTRSIEEALDNSAKVYTYFPTLANKVNAGVQGGLFLMWANDVVEDF
TTNILRKDTLDKISDVSAIIPYIGPALNISNSVRRGNFTEAFAVTGVTILLEA
FPEFTIPALGAFVIYSKVQERNEIIKTIDNCLEQRIKRWKDSYEWMMGTW
LSRIITQFNNISYQMYDSLNYQAGAIKAKIDLEYKKYSGSDKENIKSQVEN
LKNSLDVKISEAMNNINKFIRECSVTYLFKNMLPKVIDELNEFDRNTKAKLI
NLIDSHNIILVGEVDKLKAKVNNSFQNTIPFNIFSYTNNSLLKDIINEYFNNI
NDSKILSLQNRKNTLVDTSGYNAEVSEEGDVQLNPIFPFDFKLGSSGED
RGKVIVTQNENIVYNSMYESFSISFWIRINKWVSNLPGYTIIDSVKNNSG
WSIGIISNFLVFTLKQNEDSEQSINFSYDISNNAPGYNKWFFVTVTNNMM
GNMKIYINGKLIDTIKVKELTGINFSKTITFEINKIPDTGLITSDSDNINMWIR
DFYIFAKELDGKDINILFNSLQYTNVVKDYWGNDLRYNKEYYMVNIDYLN
RYMYANSRQIVFNTRRNNNDFNEGYKIIIKRIRGNTNDTRVRGGDILYFD
MTINNKAYNLFMKNETMYADNHSTEDIYAIGLREQTKDINDNIIFQIQPMN
NTYYYASQIFKSNFNGENISGICSIGTYRFRLGGDWYRHNYLVPTVKQG
NYASLLESTSTHWGFVPVSE
SEQ ID NO: 4-BoNT/D, accession number P19321, amino acid sequence
MTWPVKDFNYSDPVNDNDILYLRIPQNKLITTPVKAFMITQNIWVIPERFS
SDTNPSLSKPPRPTSKYQSYYDPSYLSTDEQKDTFLKGIIKLFKRINERDI
GKKLINYLVVGSPFMGDSSTPEDTFDFTRHTTNIAVEKFENGSWKVTNII
TPSVLIFGPLPNILDYTASLTLQGQQSNPSFEGFGTLSILKVAPEFLLTFS
DVTSNQSSAVLGKSIFCMDPVIALMHELTHSLHQLYGINIPSDKRIRPQVS
EGFFSQDGPNVQFEELYTFGGLDVEIIPQIERSQLREKALGHYKDIAKRL
NNINKTIPSSWISNIDKYKKIFSEKYNFDKDNTGNFVVNIDKFNSLYSDLT
NVMSEVVYSSQYNVKNRTHYFSRHYLPVFANILDDNIYTIRDGFNLTNKG
FNIENSGQNIERNPALQKLSSESVVDLFTKVCLRLTKNSRDDSTCIKVKN
NRLPYVADKDSISQEIFENKIITDETNVQNYSDKFSLDESILDGQVPINPEI
VDPLLPNVNMEPLNLPGEEIVFYDDITKYVDYLNSYYYLESQKLSNNVEN
ITLTTSVEEALGYSNKIYTFLPSLAEKVNKGVQAGLFLNWANEVVEDFTT
NIMKKDTLDKISDVSVIIPYIGPALNIGNSALRGNFNQAFATAGVAFLLEGF
PEFTIPALGVFTFYSSIQEREKIIKTIENCLEQRVKRWKDSYQWMVSNWL
SRITTQFNHINYQMYDSLSYQADAIKAKIDLEYKKYSGSDKENIKSQVENL
KNSLDVKISEAMNNINKFIRECSVTYLFKNMLPKVIDELNKFDLRTKTELIN
LIDSHNIILVGEVDRLKAKVNESFENTMPFNIFSYTNNSLLKDIINEYFNSIN
DSKILSLQNKKNALVDTSGYNAEVRVGDNVQLNTIYTNDFKLSSSGDKII
VNLNNNILYSAIYENSSVSFWIKISKDLTNSHNEYTIINSIEQNSGWKLCIR
NGNIEWILQDVNRKYKSLIFDYSESLSHTGYTNKWFFVTITNNIMGYMKL
YINGELKQSQKIEDLDEVKLDKTIVFGIDENIDENQMLWIRDFNIFSKELSN
EDINIVYEGQILRNVIKDYWGNPLKFDTEYYIINDNYIDRYIAPESNVLVLV
QYPDRSKLYTGNPITIKSVSDKNPYSRILNGDNIILHMLYNSRKYMIIRDTD
TIYATQGGECSQNCVYALKLQSNLGNYGIGIFSIKNIVSKNKYCSQIFSSF
RENTMLLADIYKPWRFSFKNAYTPVAVTNYETKLLSTSSFWKFISRDPG
WVE
SEQ ID NO: 5-BoNT/E1, accession number WP 003372387, amino acid sequence
MPKINSFNYNDPVNDRTILYIKPGGCQEFYKSFNIMKNIWIIPERNVIGTTP
QDFHPPTSLKNGDSSYYDPNYLQSDEEKDRFLKIVTKIFNRINNNLSGGI
LLEELSKANPYLGNDNTPDNQFHIGDASAVEIKFSNGSQDILLPNVIIMGA
EPDLFETNSSNISLRNNYMPSNHGFGSIAIVTFSPEYSFRFNDNSMNEFI
QDPALTLMHELIHSLHGLYGAKGITTKYTITQKQNPLITNIRGTNIEEFLTF
GGTDLNIITSAQSNDIYTNLLADYKKIASKLSKVQVSNPLLNPYKDVFEAK
YGLDKDASGIYSVNINKENDIFKKLYSFTEFDLATKFQVKCRQTYIGQYKY
FKLSNLLNDSIYNISEGYNINNLKVNFRGQNANLNPRIITPITGRGLVKKIIR
FCKNIVSVKGIRKSICIEINNGELFFVASENSYNDDNINTPKEIDDTVTSNN
NYENDLDQVILNFNSESAPGLSDEKLNLTIQNDAYIPKYDSNGTSDIEQH
DVNELNVFFYLDAQKVPEGENNVNLTSSIDTALLEQPKIYTFFSSEFINNV
NKPVQAALFVSWIQQVLVDFTTEANQKSTVDKIADISIVVPYIGLALNIGN
EAQKGNFKDALELLGAGILLEFEPELLIPTILVFTIKSFLGSSDNKNKVIKAI
NNALKERDEKWKEVYSFIVSNWMTKINTQFNKRKEQMYQALQNQVNAI
KTIIESKYNSYTLEEKNELTNKYDIKQIENELNQKVSIAMNNIDRFLTESSIS
YLMKLINEVKINKLREYDENVKTYLLNYIIQHGSILGESQQELNSMVTDTL
NNSIPFKLSSYTDDKILISYFNKFFKRIKSSSVLNMRYKNDKYVDTSGYDS
NININGDVYKYPTNKNQFGIYNDKLSEVNISQNDYIIYDNKYKNFSISFWV
RIPNYDNKIVNVNNEYTIINCMRDNNSGWKVSLNHNEIIWTLQDNAGINQ
KLAFNYGNANGISDYINKWIFVTITNDRLGDSKLYINGNLIDQKSILNLGNI
HVSDNILFKIVNCSYTRYIGIRYFNIFDKELDETEIQTLYSNEPNTNILKDF
WGNYLLYDKEYYLLNVLKPNNFIDRRKDSTLSINNIRSTILLANRLYSGIKV
KIQRVNNSSTNDNLVRKNDQVYINFVASKTHLFPLYADTATTNKEKTIKIS
SSGNRFNQVVVMNSVGNNCTMNFKNNNGNNIGLLGFKADTVVASTWY
YTHMRDHTNSNGCFWNFISEEHGWQEK
SEQ ID NO: 6-BoNT/F1, accession number Q57236, amino acid sequence
MPVVINSFNYNDPVNDDTILYMQIPYEEKSKKYYKAFEIMRNVWIIPERNT
IGTDPSDFDPPASLENGSSAYYDPNYLTTDAEKDRYLKTTIKLFKRINSN
PAGEVLLQEISYAKPYLGNEHTPINEFHPVTRTTSVNIKSSTNVKSSIILNL
LVLGAGPDIFENSSYPVRKLMDSGGVYDPSNDGFGSINIVTFSPEYEYTF
NDISGGYNSSTESFIADPAISLAHELIHALHGLYGARGVTYKETIKVKQAP
LMIAEKPIRLEEFLTFGGQDLNIITSAMKEKIYNNLLANYEKIATRLSRVNS
APPEYDINEYKDYFQWKYGLDKNADGSYTVNENKFNEIYKKLYSFTEIDL
ANKFKVKCRNTYFIKYGFLKVPNLLDDDIYTVSEGFNIGNLAVNNRGQNI
KLNPKIIDSIPDKGLVEKIVKFCKSVIPRKGTKAPPRLCIRVNNRELFFVAS
ESSYNENDINTPKEIDDTTNLNNNYRNNLDEVILDYNSETIPQISNQTLNT
LVQDDSYVPRYDSNGTSEIEEHNVVDLNVFFYLHAQKVPEGETNISLTS
SIDTALSEESQVYTFFSSEFINTINKPVHAALFISWINQVIRDFTTEATQKS
TFDKIADISLVVPYVGLALNIGNEVQKENFKEAFELLGAGILLEFVPELLIP
TILVFTIKSFIGSSENKNKIIKAINNSLMERETKWKEIYSWIVSNWLTRINTQ
FNKRKEQMYQALQNQVDAIKTVIEYKYNNYTSDERNRLESEYNINNIREE
LNKKVSLAMENIERFITESSIFYLMKLINEAKVSKLREYDEGVKEYLLDYIS
EHRSILGNSVQELNDLVTSTLNNSIPFELSSYTNDKILILYFNKLYKKIKDN
SILDMRYENNKFIDISGYGSNISINGDVYIYSTNRNQFGIYSSKPSEVNIAQ
NNDIIYNGRYQNFSISFWVRIPKYFNKVNLNNEYTIIDCIRNNNSGWKISL
NYNKIIWTLQDTAGNNQKLVFNYTQMISISDYINKWIFVTITNNRLGNSRIY
INGNLIDEKSISNLGDIHVSDNILFKIVGCNDTRYVGIRYFKVFDTELGKTEI
ETLYSDEPDPSILKDFWGNYLLYNKRYYLLNLLRTDKSITQNSNFLNINQ
QRGVYQKPNIFSNTRLYTGVEVIIRKNGSTDISNTDNFVRKNDLAYINVV
DRDVEYRLYADISIAKPEKIIKLIRTSNSNNSLGQIIVMDSIGNNCTMNFQN
NNGGNIGLLGFHSNNLVASSWYYNNIRKNTSSNGCFWSFISKEHGWQE
N
SEQ ID NO: 7-BoNT/G, accession number WP_039635782, amino acid sequence
MPVNIKNFNYNDPINNDDIIMMEPFNDPGPGTYYKAFRIIDRIWIVPERFT
YGFQPDQFNASTGVFSKDVYEYYDPTYLKTDAEKDKFLKTMIKLFNRINS
KPSGQRLLDMIVDAIPYLGNASTPPDKFAANVANVSINKKIIQPGAEDQIK
GLMTNLIIFGPGPVLSDNFTDSMIMNGHSPISEGFGARMMIRFCPSCLNV
FNNVQENKDTSIFSRRAYFADPALTLMHELIHVLHGLYGIKISNLPITPNTK
EFFMQHSDPVQAEELYTFGGHDPSVISPSTDMNIYNKALQNFQDIANRL
NIVSSAQGSGIDISLYKQIYKNKYDFVEDPNGKYSVDKDKFDKLYKALMF
GFTETNLAGEYGIKTRYSYFSEYLPPIKTEKLLDNTIYTQNEGENIASKNL
KTEFNGQNKAVNKEAYEEISLEHLVIYRIAMCKPVMYKNTGKSEQCIIVN
NEDLFFIANKDSFSKDLAKAETIAYNTQNNTIENNFSIDQLILDNDLSSGID
LPNENTEPFTNFDDIDIPVYIKQSALKKIFVDGDSLFEYLHAQTFPSNIENL
QLTNSLNDALRNNNKVYTFFSTNLVEKANTVVGASLFVNWVKGVIDDFT
SESTQKSTIDKVSDVSIIIPYIGPALNVGNETAKENFKNAFEIGGAAILMEFI
PELIVPIVGFFTLESYVGNKGHIIMTISNALKKRDQKWTDMYGLIVSQWLS
TVNTQFYTIKERMYNALNNQSQAIEKIIEDQYNRYSEEDKMNINIDFNDID
FKLNQSINLAINNIDDFINQCSISYLMNRMIPLAVKKLKDFDDNLKRDLLEY
IDTNELYLLDEVNILKSKVNRHLKDSIPFDLSLYTKDTILIQVENNYISNISS
NAILSLSYRGGRLIDSSGYGATMNVGSDVIFNDIGNGQFKLNNSENSNIT
AHQSKFVVYDSMFDNFSINFWVRTPKYNNNDIQTYLQNEYTIISCIKNDS
GWKVSIKGNRIIWTLIDVNAKSKSIFFEYSIKDNISDYINKWFSITITNDRLG
NANIYINGSLKKSEKILNLDRINSSNDIDFKLINCTDTTKFVWIKDFNIFGRE
LNATEVSSLYWIQSSTNTLKDFWGNPLRYDTQYYLFNQGMQNIYIKYFS
KASMGETAPRTNFNNAAINYQNLYLGLRFIIKKASNSRNINNDNIVREGD
YIYLNIDNISDESYRVYVLVNSKEIQTQLFLAPINDDPTFYDVLQIKKYYEK
TTYNCQILCEKDTKTFGLFGIGKFVKDYGYVWDTYDNYFCISQWYLRRIS
ENINKLRLGCNWQFIPVDEGWTE
SEQ ID NO: 8-BONT/DC, accession number BAM65681, amino acid sequence
MTWPVKDFNYSDPVNDNDILYLRIPQNKLITTPVKAFMITQNIWVIPERFS
SDTNPSLSKPPRPTSKYQSYYDPSYLSTDEQKDTFLKGIIKLFKRINERDI
GKKLINYLVVGSPFMGDSSTPEDTFDFTRHTTNIAVEKFENGSWKVTNII
TPSVLIFGPLPNILDYTASLTLQGQQSNPSFEGFGTLSILKVAPEFLLTFS
DVTSNQSSAVLGKSIFCMDPVIALMHELTHSLHQLYGINIPSDKRIRPQVS
EGFFSQDGPNVQFEELYTFGGSDVEIIPQIERLQLREKALGHYKDIAKRL
NNINKTIPSSWSSNIDKYKKIFSEKYNFDKDNTGNFVVNIDKFNSLYSDLT
NVMSEVVYSSQYNVKNRTHYFSKHYLPVFANILDDNIYTIINGFNLTTKGF
NIENSGQNIERNPALQKLSSESVVDLFTKVCLRLTRNSRDDSTCIQVKNN
TLPYVADKDSISQEIFESQIITDETNVENYSDNFSLDESILDAKVPTNPEAV
DPLLPNVNMEPLNVPGEEEVFYDDITKDVDYLNSYYYLEAQKLSNNVENI
TLTTSVEEALGYSNKIYTFLPSLAEKVNKGVQAGLFLNWANEVVEDFTTN
IMKKDTLDKISDVSAIIPYIGPALNIGNSALRGNFKQAFATAGVAFLLEGFP
EFTIPALGVFTFYSSIQEREKIIKTIENCLEQRVKRWKDSYQWMVSNWLS
RITTQFNHISYQMYDSLSYQADAIKAKIDLEYKKYSGSDKENIKSQVENLK
NSLDVKISEAMNNINKFIRECSVTYLFKNMLPKVIDELNKFDLKTKTELINL
IDSHNIILVGEVDRLKAKVNESFENTIPFNIFSYTNNSLLKDMINEYFNSIN
DSKILSLQNKKNTLMDTSGYNAEVRVEGNVQLNPIFPFDFKLGSSGDDR
GKVIVTQNENIVYNAMYESFSISFWIRINKWVSNLPGYTIIDSVKNNSGW
SIGIISNFLVFTLKQNENSEQDINFSYDISKNAAGYNKWFFVTITTNMMGN
MMIYINGKLIDTIKVKELTGINFSKTITFQMNKIPNTGLITSDSDNINMWIRD
FYIFAKELDDKDINILFNSLQYTNVVKDYWGNDLRYDKEYYMINVNYMNR
YMSKKGNGIVFNTRKNNNDFNEGYKIIIKRIIGNTNDTRVRGENVLYFNTT
IDNKQYSLGMYKPSRNLGTDLVPLGALDQPMDEIRKYGSFIIQPCNTFDY
YASQLFLSSNATTNRIGILSIGSYSFKLGDDYWFNHEYLIPVIKIEHYASLL
ESTSTHWVFVPASE
SEQ ID NO: 9-BoNT/F7, amino acid sequence
MPVNINNFNYNDPINNTTILYMKMPYYEDSNKYYKAFEIMDNVWIIPERNII
GKKPSDFYPPISLDSGSSAYYDPNYLTTDAEKDRFLKTVIKLFNRINSNPA
GQVLLEEIKNGKPYLGNDHTAVNEFCANNRSTSVEIKESKGTTDSMLLNL
VILGPGPNILECSTFPVRIFPNNIAYDPSEKGFGSIQLMSFSTEYEYAFND
NTDLFIADPAISLAHELIHVLHGLYGAKGVTNKKVIEVDQGALMAAEKDIKI
EEFITFGGQDLNIITNSTNQKIYDNLLSNYTAIASRLSQVNINNSALNTTYY
KNFFQWKYGLDQDSNGNYTVNISKFNAIYKKLFSFTECDLAQKFQVKNR
SNYLFHFKPFRLLDLLDDNIYSISEGFNIGSLRVNNNGQNINLNSRIVGPIP
DNGLVERFVGLCKSIVSKKGTKNSLCIKVNNRDLFFVASESSYNENGINS
PKEIDDTTITNNNYKKNLDEVILDYNSDAIPNLSSRLLNTTAQNDSYVPKY
DSNGTSEIKEYTVDKLNVFFYLYAQKAPEGESAISLTSSVNTALLDASKVY
TFFSSDFINTVNKPVQAALFISWIQQVINDFTTEATQKSTIDKIADISLVVPY
VGLALNIGNEVQKGNFKEAIELLGAGILLEFVPELLIPTILVFTIKSFINSDDS
KNKIIKAINNALRERELKWKEVYSWIVSNWLTRINTQFNKRKEQMYQALQ
NQVDGIKKIIEYKYNNYTLDEKNRLKAEYNIYSIKEELNKKVSLAMQNIDRF
LTESSISYLMKLINEAKINKLSEYDKRVNQYLLNYILENSSTLGTSSVQELN
NLVSNTLNNSIPFELSEYTNDKILISYFNRFYKRIIDSSILNMKYENNRFIDS
SGYGSNISINGDIYIYSTNRNQFGIYSSRLSEVNITQNNTIIYNSRYQNFSV
SFWVRIPKYNNLKNLNNEYTIINCMRNNNSGWKISLNYNNIIWTLQDTTG
NNQKLVFNYTQMIDISDYINKWTFVTITNNRLGHSKLYINGNLTDQKSILNL
GNIHVDDNILFKIVGCNDTRYVGIRYFKIFNMELDKTEIETLYHSEPDSTILK
DFWGNYLLYNKKYYLLNLLKPNMSVTKNSDILNINRQRGIYSKTNIFSNAR
LYTGVEVIIRKVGSTDTSNTDNFVRKNDTVYINVVDGNSEYQLYADVSTS
AVEKTIKLRRISNSNYNSNQMIIMDSIGDNCTMNFKTNNGNDIGLLGFHLN
NLVASSWYYKNIRNNTRNNGCFWSFISKEHGWQE
SEQ ID NO: 10-mrBoNT/AB (BONT/ABMY), amino acid sequence

| | |
|---|---|
| MPFVNKQFNYKDPVNGVDIAYIKIPNAGQMQPVKAFKIHNKIWVIPERD | 49 |
| TFTNPEEGDLNPPPEAKQVPVSYYDSTYLSTDNEKDNYLKGVTKLFERIY | 99 |
| STDLGRMLLTSIVRGIPFWGGSTIDTELKVIDTNCINVIQPDGSYRSEEL | 149 |
| NLVIIGPSADIIQFECKSFGHEVLNLTRNGYGSTQYIRFSPDFTFGFEES | 199 |
| LEVDTNPLLGAGKFATDPAVTLAHELIHAGHRLYGIAINPNRVFKVNTNA | 249 |
| YYEMSGLEVSFEELRTFGGHDAKFIDSLQENEFRLYYYNKFKDIASTLNK | 299 |
| AKSIVGTTASLQYMKNVFKEKYLLSEDTSGKFSVDKLKFDKLYKMLTEIY | 349 |
| TEDNFVKFFKVLNRKTYLNFDKAVFKINIVPKVNYTIYDGFNLRNTNLAA | 399 |
| NFNGQNTEINNMNFTKLKNFTGLFEFYKLLCVRGIITSKTKSLDKGYNKA | 449 |
| LNDLCIKVNNWDLFFSPSEDNFTNDLNKGEEITSDTNIEAAEENISLDLI | 499 |
| QQYYLTFNFDNEPENISIENLSSDIIGQLELMPNIERFPNGKKYELDKYT | 549 |
| MFHYLRAQEFEHGKSRIALTNSVNEALLNPSRVYTFFSSDYVKKVNKATE | 599 |
| AAMFLGWVEQLVYDFTDETSEVSTTDKIADITIIIPYIGPALNIGNMLYK | 649 |
| DDFVGALIFSGAVILLEFIPEIAIPVLGTFALVSYIANKVLTVQTIDNAL | 699 |
| SKRNEKWDEVYKYIVTNWLAKVNTQIDLIRKKMKEALENQAEATKAIINY | 749 |
| QYNQYTEEEKNNINFNIDDLSSKLNESINKAMININKFLNQCSVSYLMNS | 799 |
| MIPYGVKRLEDFDASLKDALLKYIYDNRGTLIGQVDRLKDKVNNTLSTDI | 849 |
| PFQLSKYVDNQRLLSTFTEYIKNILNNIILNLRYKDNNLIDLSGYGAKVE | 899 |
| VYDGVELNDKNQFKLTSSANSKIRVTQNQNIIFNSVFLDFSVSFWIRIPK | 949 |
| YKNDGIQNYIHNEYTIINCMKNNSGWKISIRGNRIIWTLIDINGKTKSVF | 999 |
| FEYNIREDISEYINRWFFVTITNNLNNAKIYINGKLESNTDIKDIREVIA | 1049 |
| NGEIIFKLDGDIDRTQFIWMKYFSIFNTELSQSNIEERYKIQSYSEYLKD | 1099 |
| FWGNPLMYNKEYYMFNAGNKNSYIKLKKDSPVGEILTRSKYNQNSKYINY | 1149 |
| RDLYIGEKFIIRRKSNSQSINDDIVRKEDYIYLDFFNLNQEWRVYTYKYF | 1199 |
| KKEEMKLFLAPIYDSDEFYNTIQIKEYDEQPTYSCQLLFKKDEESTDEIG | 1249 |
| LIGIHRFYESGIVFEEYKDYFCISKWYLKEVKRKPYNLKLGCNWQFIPKD | 1299 |
| EGWTE | 1304 |

SEQ ID NO: 11-BoNT/X, amino acid sequence (GenBank: BAQ12790.1)
MKLEINKFNYNDPIDGINVITMRPPRHSDKINKGKGPFKAFQVIKNIWIVPERY
NFTNNTNDLNIPSEPIMEADAIYNPNYLNTPSEKDEFLQGVIKVLERIKSKPEG
EKLLELISSSIPLPLVSNGALTLSDNETIAYQENNNIVSNLQANLVIYGPGPDIA
NNATYGLYSTPISNGEGTLSEVSFSPFYLKPFDESYGNYRSLVNIVNKFVKR
EFAPDPASTLMHELVHVTHNLYGISNRNFYYNFDTGKIETSRQQNSLIFEELL
TFGGIDSKAISSLIIKKIIETAKNNYTTLISERLNTVTVENDLLKYIKNKIPVQGRL
GNFKLDTAEFEKKLNTILFVLNESNLAQRFSILVRKHYLKERPIDPIYVNILDDN
SYSTLEGFNISSQGSNDFQGQLLESSYFEKIESNALRAFIKICPRNGLLYNAIY
RNSKNYLNNIDLEDKKTTSKTNVSYPCSLLNGCIEVENKDLFLISNKDSLNDIN
LSEEKIKPETTVFFKDKLPPQDITLSNYDFTEANSIPSISQQNILERNEELYEPI
RNSLFEIKTIYVDKLTTFHFLEAQNIDESIDSSKIRVELTDSVDEALSNPNKVYS
PFKNMSNTINSIETGITSTYIFYQWLRSIVKDFSDETGKIDVIDKSSDTLAIVPYI
GPLLNIGNDIRHGDFVGAIELAGITALLEYVPEFTIPILVGLEVIGGELAREQVE
AIVNNALDKRDQKWAEVYNITKAQWWGTIHLQINTRLAHTYKALSRQANAIK
MNMEFQLANYKGNIDDKAKIKNAISETEILLNKSVEQAMKNTEKFMIKLSNSY
LTKEMIPKVQDNLKNFDLETKKTLDKFIKEKEDILGTNLSSSLRRKVSIRLNKNI
AFDINDIPFSEFDDLINQYKNEIEDYEVLNLGAEDGKIKDLSGTTSDINIGSDIE
LADGRENKAIKIKGSENSTIKIAMNKYLRFSATDNFSISFWIKHPKPTNLLNNGI
EYTLVENFNQRGWKISIQDSKLIWYLRDHNNSIKIVTPDYIAFNGWNLITITNN
RSKGSIVYVNGSKIEEKDISSIWNTEVDDPIIFRLKNNRDTQAFTLLDQFSIYR
KELNQNEVVKLYNYYFNSNYIRDIWGNPLQYNKKYYLQTQDKPGKGLIREY
WSSFGYDYVILSDSKTITFPNNIRYGALYNGSKVLIKNSKKLDGLVRNKDFIQL
EIDGYNMGISADRFNEDTNYIGTTYGTTHDLTTDFEIIQRQEKYRNYCQLKTP
YNIFHKSGLMSTETSKPTFHDYRDWVYSSAWYFQNYENLNLRKHTKTNWY
FIPKDEGWDED
SEQ ID NO: 12-Polypeptide Sequence of TeNT-UniProt P04958
MPITINNFRYSDPVNNDTIIMMEPPYCKGLDIYYKAFKITDRIWIVPERYEFGT
KPEDFNPPSSLIEGASEYYDPNYLRTDSDKDRFLQTMVKLFNRIKNNVAGEA
LLDKIINAIPYLGNSYSLLDKFDTNSNSVSFNLLEQDPSGATTKSAMLTNLIIFG
PGPVLNKNEVRGIVLRVDNKNYFPCRDGFGSIMQMAFCPEYVPTFDNVIENI
TSLTIGKSKYFQDPALLLMHELIHVLHGLYGMQVSSHEIIPSKQEIYMQHTYPI
SAEELFTFGGQDANLISIDIKNDLYEKTLNDYKAIANKLSQVTSCNDPNIDIDS
YKQIYQQKYQFDKDSNGQYIVNEDKFQILYNSIMYGFTEIELGKKFNIKTRLSY
FSMNHDPVKIPNLLDDTIYNDTEGFNIESKDLKSEYKGQNMRVNTNAFRNVD
GSGLVSKLIGLCKKIIPPTNIRENLYNRTASLTDLGGELCIKIKNEDLTFIAEKNS
FSEEPFQDEIVSYNTKNKPLNFNYSLDKIIVDYNLQSKITLPNDRTTPVTKGIP
YAPEYKSNAASTIEIHNIDDNTIYQYLYAQKSPTTLQRITMTNSVDDALINSTKI
YSYFPSVISKVNQGAQGILFLQWVRDIIDDFTNESSQKTTIDKISDVSTIVPYIG
PALNIVKQGYEGNFIGALETTGVVLLLEYIPEITLPVIAALSIAESSTQKEKIIKTI
DNFLEKRYEKWIEVYKLVKAKWLGTVNTQFQKRSYQMYRSLEYQVDAIKKII
DYEYKIYSGPDKEQIADEINNLKNKLEEKANKAMININIFMRESSRSFLVNQMI
NEAKKQLLEFDTQSKNILMQYIKANSKFIGITELKKLESKINKVFSTPIPFSYSK
NLDCWVDNEEDIDVILKKSTILNLDINNDIISDISGENSSVITYPDAQLVPGING
KAIHLVNNESSEVIVHKAMDIEYNDMFNNFTVSFWLRVPKVSASHLEQYGTN
EYSIISSMKKHSLSIGSGWSVSLKGNNLIWTLKDSAGEVRQITFRDLPDKFNA
YLANKWVFITITNDRLSSANLYINGVLMGSAEITGLGAIREDNNITLKLDRCNN
NNQYVSIDKFRIFCKALNPKEIEKLYTSYLSITFLRDFWGNPLRYDTEYYLIPV
ASSSKDVQLKNITDYMYLTNAPSYTNGKLNIYYRRLYNGLKFIIKRYTPNNEID
SFVKSGDFIKLYVSYNNNEHIVGYPKDGNAFNNLDRILRVGYNAPGIPLYKKM
EAVKLRDLKTYSVQLKLYDDKNASLGLVGTHNGQIGNDPNRDILIASNWYFN
HLKDKILGCDWYFVPTDEGWTND
SEQ ID NO: 13-Polypeptide Sequence of BoNT/A-UniProt P10845
MPFVNKQFNYKDPVNGVDIAYIKIPNVGQMQPVKAFKIHNKIWVIPERDTFTN
PEEGDLNPPPEAKQVPVSYYDSTYLSTDNEKDNYLKGVTKLFERIYSTDLGR
MLLTSIVRGIPFWGGSTIDTELKVIDTNCINVIQPDGSYRSEELNLVIIGPSADII
QFECKSFGHEVLNLTRNGYGSTQYIRFSPDFTFGFEESLEVDTNPLLGAGKF
ATDPAVTLAHELIHAGHRLYGIAINPNRVFKVNTNAYYEMSGLEVSFEELRTF
GGHDAKFIDSLQENEFRLYYYNKFKDIASTLNKAKSIVGTTASLQYMKNVFKE
KYLLSEDTSGKFSVDKLKFDKLYKMLTEIYTEDNFVKFFKVLNRKTYLNFDKA
VFKINIVPKVNYTIYDGFNLRNTNLAANFNGQNTEINNMNFTKLKNFTGLFEF
YKLLCVRGIITSKTKSLDKGYNKALNDLCIKVNNWDLFFSPSEDNFTNDLNKG
EEITSDTNIEAAEENISLDLIQQYYLTFNFDNEPENISIENLSSDIIGQLELMPNI
ERFPNGKKYELDKYTMFHYLRAQEFEHGKSRIALTNSVNEALLNPSRVYTFF
SSDYVKKVNKATEAAMFLGWVEQLVYDFTDETSEVSTTDKIADITIIIPYIGPA
LNIGNMLYKDDFVGALIFSGAVILLEFIPEIAIPVLGTFALVSYIANKVLTVQTID
NALSKRNEKWDEVYKYIVTNWLAKVNTQIDLIRKKMKEALENQAEATKAIINY
QYNQYTEEEKNNINFNIDDLSSKLNESINKAMININKFLNQCSVSYLMNSMIP
YGVKRLEDFDASLKDALLKYIYDNRGTLIGQVDRLKDKVNNTLSTDIPFQLSK
YVDNQRLLSTFTEYIKNIINTSILNLRYESNHLIDLSRYASKINIGSKVNFDPIDK
NQIQLFNLESSKIEVILKNAIVYNSMYENFSTSFWIRIPKYFNSISLNNEYTIINC
MENNSGWKVSLNYGEIIWTLQDTQEIKQRVVFKYSQMINISDYINRWIFVTIT
NNRLNNSKIYINGRLIDQKPISNLGNIHASNNIMFKLDGCRDTHRYIWIKYFNL
FDKELNEKEIKDLYDNQSNSGILKDFWGDYLQYDKPYYMLNLYDPNKYVDV
NNVGIRGYMYLKGPRGSVMTTNIYLNSSLYRGTKFIIKKYASGNKDNIVRNND
RVYINVVVKNKEYRLATNASQAGVEKILSALEIPDVGNLSQVVVMKSKNDQGI
TNKCKMNLQDNNGNDIGFIGFHQFNNIAKLVASNWYNRQIERSSRTLGCSW
EFIPVDDGWGERPL
SEQ ID NO: 14-Nucleotide Sequence of mrBoNT/A
ATGCCATTCGTCAACAAGCAATTCAACTACAAAGACCCAGTCAACGGCGT
CGACATCGCATACATCAAGATTCCGAACGCCGGTCAAATGCAGCCGGTT
AAGGCTTTTAAGATCCACAACAAGATTTGGGTTATCCCGGAGCGTGACAC
CTTCACGAACCCGGAAGAAGGCGATCTGAACCCGCCACCGGAAGCGAA
GCAAGTCCCTGTCAGCTACTACGATTCGACGTACCTGAGCACGGATAAC
GAAAAAGATAACTACCTGAAAGGTGTGACCAAGCTGTTCGAACGTATCTA
CAGCACGGATCTGGGTCGCATGCTGCTGACTAGCATTGTTCGCGGTATC
CCGTTCTGGGGTGGTAGCACGATTGACACCGAACTGAAGGTTATCGACA
CTAACTGCATTAACGTTATTCAACCGGATGGTAGCTATCGTAGCGAAGAG
CTGAATCTGGTCATCATTGGCCCGAGCGCAGACATTATCCAATTCGAGT
GCAAGAGCTTTGGTCACGAGGTTCTGAATCTGACCCGCAATGGCTATGG
TAGCACCCAGTACATTCGTTTTTCGCCGGATTTTACCTTCGGCTTTGAAG
AGAGCCTGGAGGTTGATACCAATCCGTTGCTGGGTGCGGGCAAATTCGC
TACCGATCCGGCTGTCACGCTGGCCCATGAACTGATCCACGCAGGCCAC
CGCCTGTACGGCATTGCCATCAACCCAAACCGTGTGTTCAAGGTTAATAC
GAATGCATACTACGAGATGAGCGGCCTGGAAGTCAGCTTCGAAGAACTG
CGCACCTTCGGTGGCCATGACGCTAAATTCATTGACAGCTTGCAAGAGA
ATGAGTTCCGTCTGTACTACTATAACAAATTCAAAGACATTGCAAGCACG
TTGAACAAGGCCAAAAGCATCGTTGGTACTACCGCGTCGTTGCAGTATAT
GAAGAATGTGTTTAAAGAGAAGTACCTGCTGTCCGAGGATACCTCCGGC
AAGTTTAGCGTTGATAAGCTGAAGTTTGACAAACTGTACAAGATGCTGAC
CGAGATTTACACCGAGGACAACTTTGTGAAATTCTTCAAAGTGTTGAATC
GTAAAACCTATCTGAATTTTGACAAAGCGGTTTTCAAGATTAACATCGTGC
CGAAGGTGAACTACACCATCTATGACGGTTTTAACCTGCGTAACACCAAC
CTGGCGGCGAACTTTAACGGTCAGAATACGGAAATCAACAACATGAATTT
CACGAAGTTGAAGAACTTCACGGGTCTGTTCGAGTTCTATAAGCTGCTGT
GCGTGCGCGGTATCATCACCAGCAAAACCAAAAGCCTGGACAAAGGCTA
CAACAAGGCGCTGAATGACCTGTGCATTAAGGTAAACAATTGGGATCTGT
TCTTTTCGCCATCCGAAGATAATTTTACCAACGACCTGAACAAGGGTGAA
GAAATCACCAGCGATACGAATATTGAAGCAGCGGAAGAGAATATCAGCC
TGGATCTGATCCAGCAGTACTATCTGACCTTTAACTTCGACAATGAACCG
GAGAACATTAGCATTGAGAATCTGAGCAGCGACATTATCGGTCAGCTGG
AACTGATGCCGAATATCGAACGTTTCCCGAACGGCAAAAAGTACGAGCT
GGACAAGTACACTATGTTCCATTACCTGCGTGCACAGGAGTTTGAACAC
GGTAAAAGCCGTATCGCGCTGACCAACAGCGTTAACGAGGCCCTGCTGA
ACCCGAGCCGTGTCTATACCTTCTTCAGCAGCGACTATGTTAAGAAAGTG
AACAAAGCCACTGAGGCCGCGATGTTCCTGGGCTGGGTGGAACAGCTG
GTATATGACTTCACGGACGAGACGAGCGAAGTGAGCACTACCGACAAAA
TTGCTGATATTACCATCATTATCCCGTATATTGGTCCGGCACTGAACATT
GGCAACATGCTGTACAAAGACGATTTTGTGGGTGCCCTGATCTTCTCCG
GTGCCGTGATTCTGCTGGAGTTCATTCCGGAGATTGCGATCCCGGTGTT
GGGTACCTTCGCGCTGGTGTCCTACATCGCGAATAAGGTTCTGACGGTT
CAGACCATCGATAACGCGCTGTCGAAACGTAATGAAAAATGGGACGAGG
TTTACAAATACATTGTTACGAATTGGCTGGCGAAAGTCAATACCCAGATC
GACCTGATCCGTAAGAAAATGAAAGAGGCGCTGGAGAATCAGGCGGAG
GCCACCAAAGCAATTATCAACTACCAATACAACCAGTACACGGAAGAAGA
GAAGAATAACATTAACTTCAATATCGATGATTTGAGCAGCAAGCTGAATG
AATCTATCAACAAAGCGATGATCAATATCAACAAGTTTTTGAATCAGTGTA
GCGTTTCGTACCTGATGAATAGCATGATTCCGTATGGCGTCAAACGTCTG
GAGGACTTCGACGCCAGCCTGAAAGATGCGTTGCTGAAATACATTTACG
ACAATCGTGGTACGCTGATTGGCCAAGTTGACCGCTTGAAAGACAAAGT
TAACAATACCCTGAGCACCGACATCCCATTTCAACTGAGCAAGTATGTTG
ATAATCAACGTCTGTTGAGCACTTTCACCGAGTATATCAAAAACATCATCA
ATACTAGCATTCTGAACCTGCGTTACGAGAGCAAGCATCTGATTGATCTG
AGCCGTTATGCTAGCAAGATCAACATCGGTAGCAAGGTCAATTTTGACCC
GATCGATAAGAACCAGATCCAGCTGTTTAATCTGGAATCGAGCAAAATTG
AGGTTATCCTGAAAAAGGCCATTGTCTACAACTCCATGTACGAGAATTTC
TCCACCAGCTTCTGGATTCGCATCCCGAAATACTTCAACAAGATTAGCCT
GAACAACGAGTATACTATCATCAACTGTATGGAGAACAACAGCGGTTGGA
AGGTGTCTCTGAACTATGGTGAGATCATTTGGACCTTGCAGGACACCAAA
GAGATCAAGCAGCGCGTCGTGTTCAAGTACTCTCAAATGATCAACATTTC
CGATTACATTAATCGTTGGATCTTCGTGACCATTACGAATAACCGTCTGA
ATAAGAGCAAGATTTACATCAATGGTCGCTTGATCGATCAGAAACCGATT
AGCAACCTGGGTAATATCCACGCAAGCAACAAGATTATGTTCAAATTGGA
CGGTTGCCGCGATACCCATCGTTATATCTGGATCAAGTATTTCAACCTGT
TTGATAAAGAACTGAATGAGAAGGAGATCAAAGATTTGTATGACAACCAA
TCTAACAGCGGCATTTTGAAGGACTTCTGGGGCGATTATCTGCAATACGA
TAAGCCGTACTATATGCTGAACCTGTATGATCCGAACAAATATGTGGATG
TCAATAATGTGGGTATTCGTGGTTACATGTATTTGAAGGGTCCGCGTGGC
AGCGTTATGACGACCAACATTTACCTGAACTCTAGCCTGTACCGTGGTAC
GAAATTCATCATTAAGAAATATGCCAGCGGCAACAAAGATAACATTGTGC
GTAATAACGATCGTGTCTACATCAACGTGGTCGTGAAGAATAAAGAGTAC
CGTCTGGCGACCAACGCTTCGCAGGCGGGTGTTGAGAAAATTCTGAGC
GCGTTGGAGATCCCTGATGTCGGTAATCTGAGCCAAGTCGTGGTTATGA
AGAGCAAGAACGACAAGGGTATCACTAACAAGTGCAAGATGAACCTGCA
AGACAACAATGGTAACGACATCGGCTTTATTGGTTTCCACCAGTTCAACA
ATATTGCTAAACTGGTAGCGAGCAATTGGTACAATCGTCAGATTGAGCGC
AGCAGCcGTACTTTGGGCTGTAGCTGGGAGTTTATCCCGGTCGATGATG
GTTGGGGCGAACGTCCGCTG
SEQ ID NO: 15-Polypeptide Sequence of mrBoNT/A
MPFVNKQFNYKDPVNGVDIAYIKIPNAGQMQPVKAFKIHNKIWVIPERDTFTN
PEEGDLNPPPEAKQVPVSYYDSTYLSTDNEKDNYLKGVTKLFERIYSTDLGR
MLLTSIVRGIPFWGGSTIDTELKVIDTNCINVIQPDGSYRSEELNLVIIGPSADII
QFECKSFGHEVLNLTRNGYGSTQYIRFSPDFTFGFEESLEVDTNPLLGAGKF
ATDPAVTLAHELIHAGHRLYGIAINPNRVFKVNTNAYYEMSGLEVSFEELRTF
GGHDAKFIDSLQENEFRLYYYNKFKDIASTLNKAKSIVGTTASLQYMKNVFKE
KYLLSEDTSGKFSVDKLKFDKLYKMLTEIYTEDNFVKFFKVLNRKTYLNFDKA
VFKINIVPKVNYTIYDGFNLRNTNLAANFNGQNTEINNMNFTKLKNFTGLFEF
YKLLCVRGIITSKTKSLDKGYNKALNDLCIKVNNWDLFFSPSEDNFTNDLNKG
EEITSDTNIEAAEENISLDLIQQYYLTFNFDNEPENISIENLSSDIIGQLELMPNI
ERFPNGKKYELDKYTMFHYLRAQEFEHGKSRIALTNSVNEALLNPSRVYTFF
SSDYVKKVNKATEAAMFLGWVEQLVYDFTDETSEVSTTDKIADITIIIPYIGPA
LNIGNMLYKDDFVGALIFSGAVILLEFIPEIAIPVLGTFALVSYIANKVLTVQTID
NALSKRNEKWDEVYKYIVTNWLAKVNTQIDLIRKKMKEALENQAEATKAIINY
QYNQYTEEEKNNINFNIDDLSSKLNESINKAMININKFLNQCSVSYLMNSMIP
YGVKRLEDFDASLKDALLKYIYDNRGTLIGQVDRLKDKVNNTLSTDIPFQLSK
YVDNQRLLSTFTEYIKNIINTSILNLRYESKHLIDLSRYASKINIGSKVNFDPIDK
NQIQLFNLESSKIEVILKKAIVYNSMYENFSTSFWIRIPKYFNKISLNNEYTIINC
MENNSGWKVSLNYGEIIWTLQDTKEIKQRVVFKYSQMINISDYINRWIFVTITN
NRLNKSKIYINGRLIDQKPISNLGNIHASNKIMFKLDGCRDTHRYIWIKYFNLF
DKELNEKEIKDLYDNQSNSGILKDFWGDYLQYDKPYYMLNLYDPNKYVDVN
NVGIRGYMYLKGPRGSVMTTNIYLNSSLYRGTKFIIKKYASGNKDNIVRNNDR
VYINVVVKNKEYRLATNASQAGVEKILSALEIPDVGNLSQVVVMKSKNDKGIT
NKCKMNLQDNNGNDIGFIGFHQFNNIAKLVASNWYNRQIERSSRTLGCSWE
FIPVDDGWGERPL
SEQ ID NO: 16-Polypeptide Sequence of BoNT/E-UniProt Q00496
MPKINSFNYNDPVNDRTILYIKPGGCQEFYKSFNIMKNIWIIPERNVIGTTPQD
FHPPTSLKNGDSSYYDPNYLQSDEEKDRFLKIVTKIFNRINNNLSGGILLEELS
KANPYLGNDNTPDNQFHIGDASAVEIKFSNGSQDILLPNVIIMGAEPDLFETN
SSNISLRNNYMPSNHRFGSIAIVTFSPEYSFRFNDNCMNEFIQDPALTLMHEL
IHSLHGLYGAKGITTKYTITQKQNPLITNIRGTNIEEFLTFGGTDLNIITSAQSND
IYTNLLADYKKIASKLSKVQVSNPLLNPYKDVFEAKYGLDKDASGIYSVNINKF
NDIFKKLYSFTEFDLRTKFQVKCRQTYIGQYKYFKLSNLLNDSIYNISEGYNIN
NLKVNFRGQNANLNPRIITPITGRGLVKKIIRFCKNIVSVKGIRKSICIEINNGEL
FFVASENSYNDDNINTPKEIDDTVTSNNNYENDLDQVILNFNSESAPGLSDE
KLNLTIQNDAYIPKYDSNGTSDIEQHDVNELNVFFYLDAQKVPEGENNVNLT
SSIDTALLEQPKIYTFFSSEFINNVNKPVQAALFVSWIQQVLVDFTTEANQKST
VDKIADISIVVPYIGLALNIGNEAQKGNFKDALELLGAGILLEFEPELLIPTILVFT
IKSFLGSSDNKNKVIKAINNALKERDEKWKEVYSFIVSNWMTKINTQFNKRKE
QMYQALQNQVNAIKTIIESKYNSYTLEEKNELTNKYDIKQIENELNQKVSIAMN
NIDRFLTESSISYLMKIINEVKINKLREYDENVKTYLLNYIIQHGSILGESQQELN
SMVTDTLNNSIPFKLSSYTDDKILISYFNKFFKRIKSSSVLNMRYKNDKYVDTS
GYDSNININGDVYKYPTNKNQFGIYNDKLSEVNISQNDYIIYDNKYKNFSISFW
VRIPNYDNKIVNVNNEYTIINCMRDNNSGWKVSLNHNEIIWTFEDNRGINQKL
AFNYGNANGISDYINKWIFVTITNDRLGDSKLYINGNLIDQKSILNLGNIHVSD
NILFKIVNCSYTRYIGIRYFNIFDKELDETEIQTLYSNEPNTNILKDFWGNYLLY
DKEYYLLNVLKPNNFIDRRKDSTLSINNIRSTILLANRLYSGIKVKIQRVNNSST
NDNLVRKNDQVYINFVASKTHLFPLYADTATTNKEKTIKISSSGNRFNQVVVM
NSVGNCTMNFKNNNGNNIGLLGFKADTVVASTWYYTHMRDHTNSNGCFW
NFISEEHGWQEK
SEQ ID NO: 17-Substance P
RPKPQQFFGLM

### EXAMPLES

The invention will now be described, by way of example only, with reference to the following Examples. The Examples serve to illustrate particular embodiments of the invention, and do not limit the scope of the invention defined in the claims in any way.

The objective of this study was to evaluate the efficacy of a clostridial neurotoxin administered via the intradermal route in the Rhino mice model of skin conditions. Dysport (BoNT/A) was chosen as the test clostridial neurotoxin. Briefly, animals were injected (as a single administration on day 1) via the intradermal route (four sites of injection) with vehicle (saline) or Dysport at three escalating doses under anaesthesia (0.1, 0.3 and 1 Unit of Dysport per mouse). A reference group was administered Adapalene gel 0.1% by topical application, daily for 15 days. During this period, animals were observed daily. The body weight and the thickness of the dorsal skin were measured on days 1, 5, 10 and 15; macroscopic observations (erythema and scaling scoring) were achieved on mice at the same time. Sebum samples were collected on day 15 only. On day 15, animals were ethically euthanized and skin samples were collected for histology Hematoxylin Eosin Safran (HES, for structural/anatomical analysis of e.g. utricule size) and Ki67 (proliferation marker) staining, ELISA and lipidomic assays.

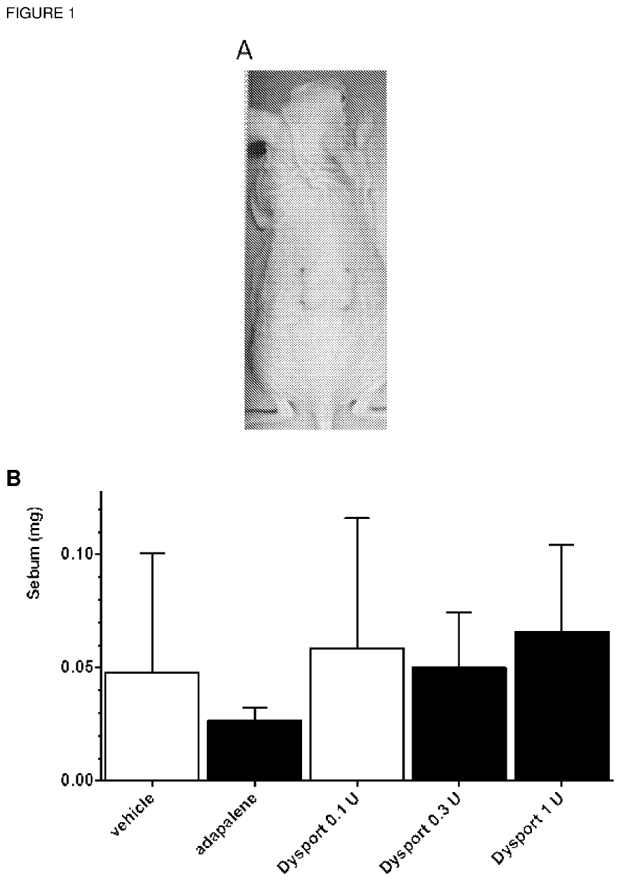

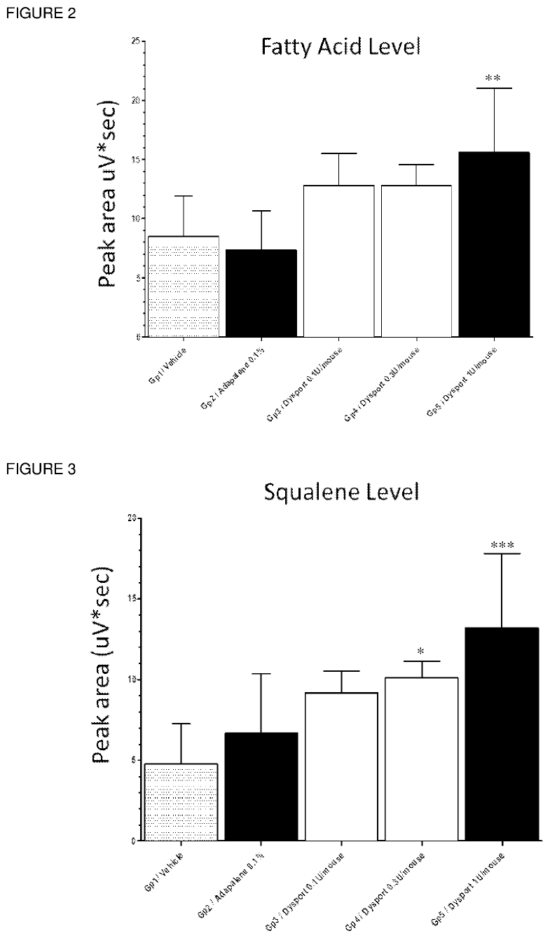

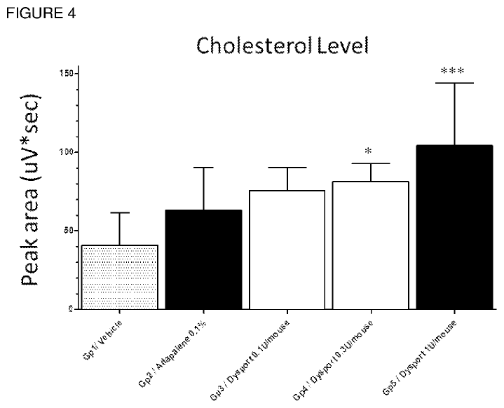

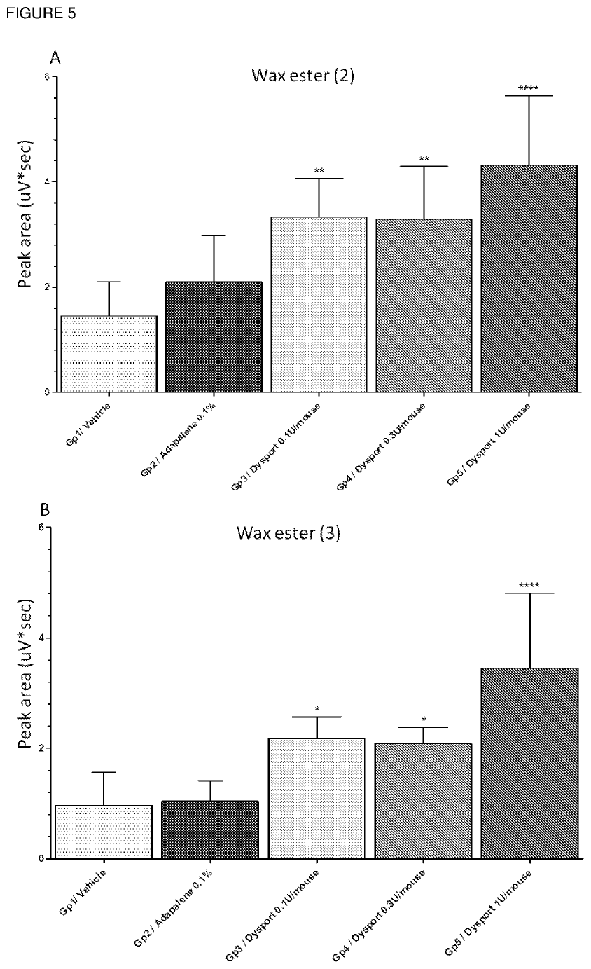

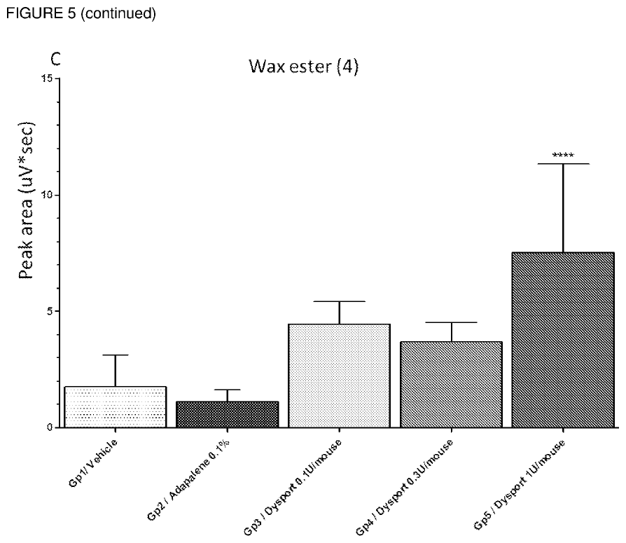

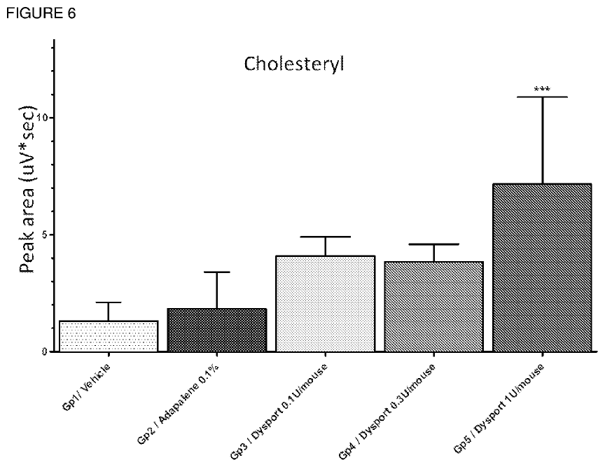

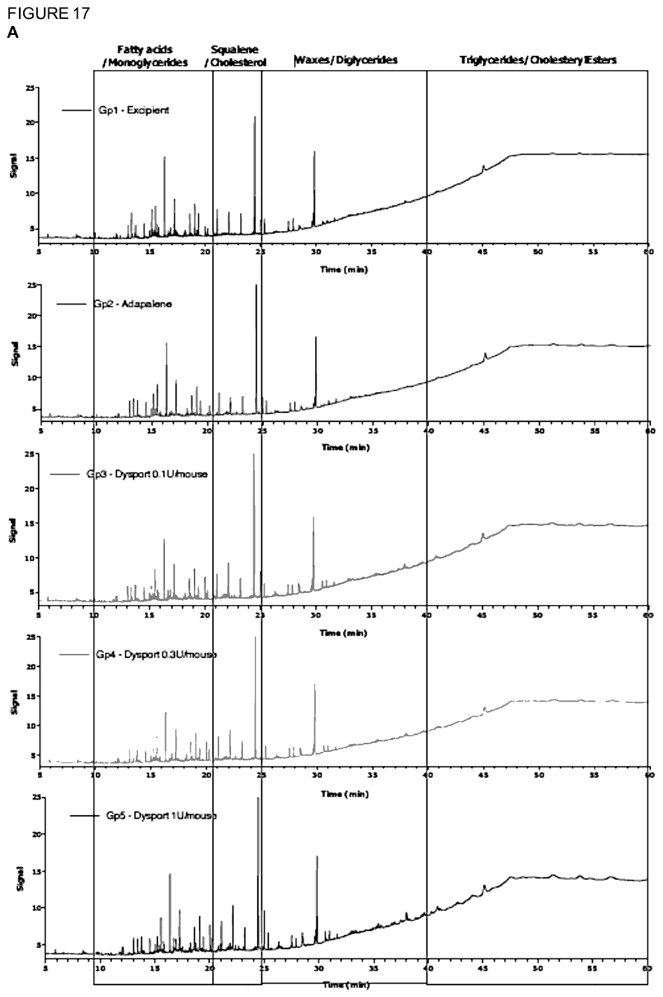

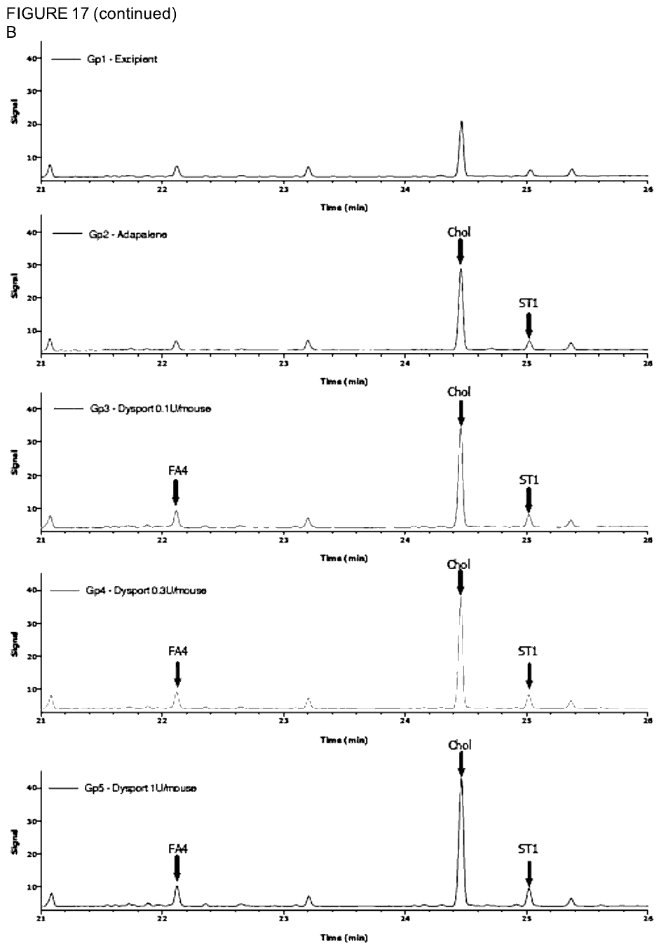

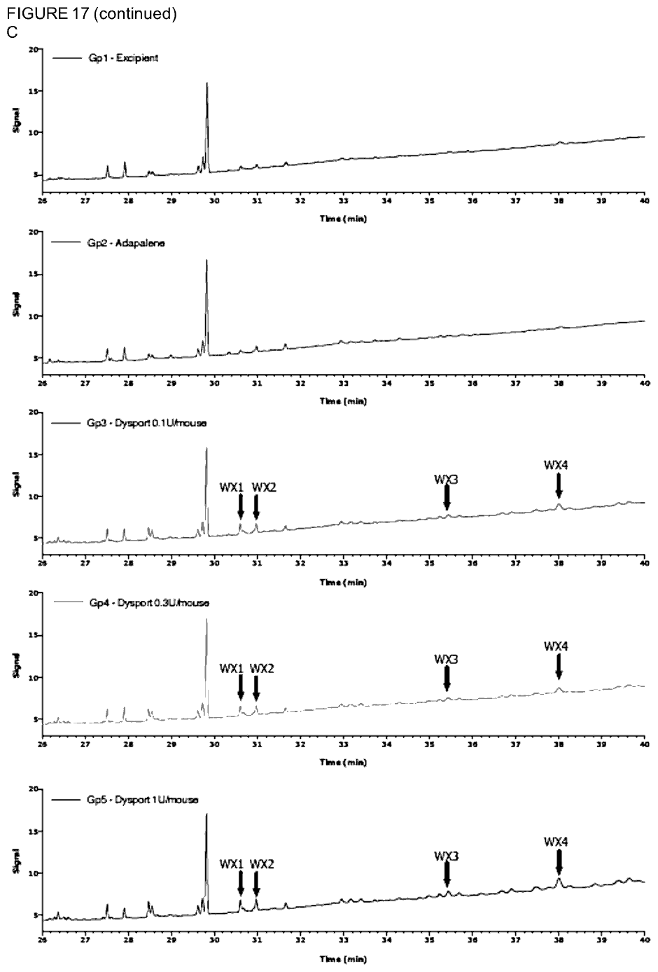

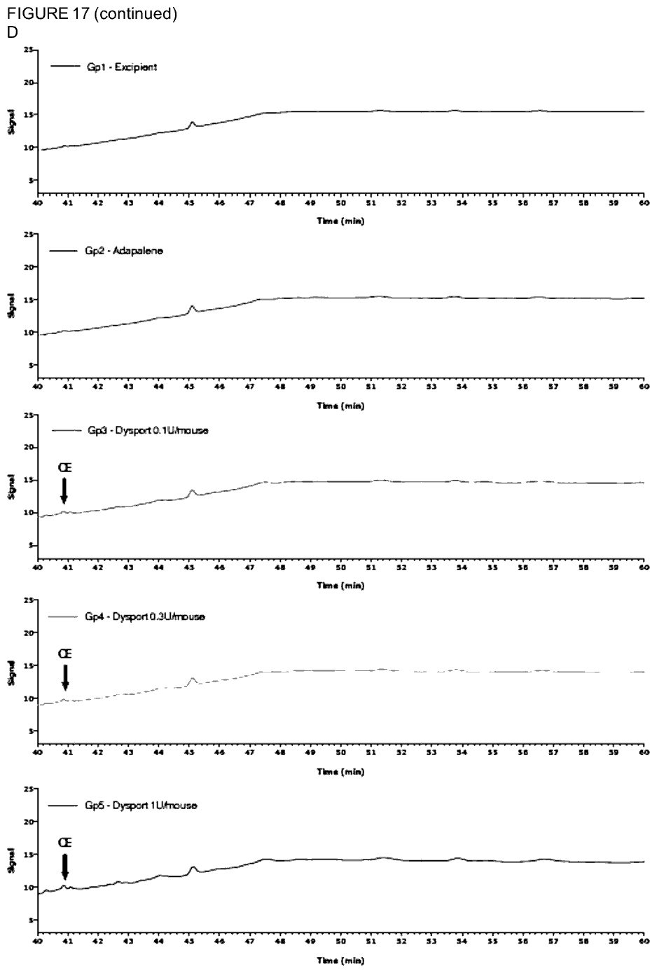

In conclusion, Dysport administered at the doses 0.1 U and 0.3 U per mouse via the intradermal route was well tolerated. The highest dose of Dysport (1 U per mouse) induced a transient loss of muscle tone associated with a slight body weight loss (which was due to diffusion of Dysport away from the intradermal injection site); mice recovered a good health status after 8 days. Dysport did not show any significant effect on utricles surface area (see FIG. 10) but induced a significant anti-inflammatory effect on dermis at the two highest doses (see FIG. 12). The inflammation of the dermis (back skin area) was assessed for each mouse on HES slices by counting the number of nuclei which is a marker of inflammatory infiltrate. Moreover, Dysport did not show any side effects commonly observed with local retinoid treatments (e.g. adapalene), as well as epidermis hyperplasia and dermis inflammation. No change in IL-1 alpha cytokine levels was seen with Dysport (see FIG. 16), whereas a dose-dependent increase of Substance P concentration in the dorsal skin was recorded (see FIG. 17), suggesting that Dysport induces neuropeptide accumulation in tissue for example by by inhibiting exocytosis mechanisms. Lipidomic analysis showed that Dysport significantly increased certain (desirable) lipid species, which present skin protective properties, such as fatty acid (FIG. 2), squalene (FIG. 3), cholesterol (FIG. 4), wax ester (FIG. 5), and cholesteryl (FIG. 6). The overall sebum levels did not change significantly (FIG. 1B).

### Materials and Methods

### Mice

Strain used: RHJ/LeJ (e.g. Rhino Mice); Sex: female; age: 6-8 weeks at reception; weight: approx. 20 g at arrival; number: 30 animals+2 spare animals.

This mouse strain is known to be a suitable model for evaluating efficacy and pharmacodynamics of new drugs for skin conditions. There is a substantial amount of historical data available in the literature for comparison purposes.

Supplier: Animals were obtained from The Jackson Laboratory, 600 Main Street, Bar Harbor ME 04609, USA, and imported by Charles River Laboratories, BP 0109, 69592 L'Arbresle Cedex, France.

Housing: Animals were housed in cages by group of 6 animals. Enrichment were done in cages. Equipment and animal houses were cleaned at regular intervals. Cages were identified with a label containing, as a minimum, the study number, and the animals identification. Minimum and maximum room temperatures (target: 22±2° C.) and humidity (target: SO±20%) were daily recorded and retained in the raw data. The light/dark cycle was 12/12 h.

Test Item

BoNT/A (Dysport) was stored at +4° C. until use. Adapalene gel 0.1% was obtained from commercial source (Differine®, Galderma France).

Dysport (552 U Dysport) reconstitution (in 1 ml sterile saline=stock solution) and further dilutions (in 5 ml siliconized tubes, Vacutainer, BD Biosciences) were done in sterile saline, as follows:

| | Final neurotoxin | | Concentration of | | | |
|---|---|---|---|---|---|---|
| | concentration | | neurotoxin solution | Neurotoxin | Saline | Final |
| Group | (units/mL) | Initial solution | (units/mL) | solution (μL) | (μL) | (μL) |
| 3 | 1.25 | Diluted stock | 55.2 | 36 | 1564 | 1600 |
| | | solution | | | | |
| 4 | 3.75 | Diluted stock | 55.2 | 108 | 1492 | 1600 |
| | | solution | | | | |
| 5 | 12.5 | Stock solution | 552 | 36 | 1564 | 1600 |

### Treatment

Dysport (test item) or saline (negative control) was injected via the intradermal route into 4 sites on the back (see FIG. 1). The reference compound Adapalene (positive control) was applied by topical route (back skin). All administrations were done on the same day. The following administration dosages were assessed as followed:

| | Number of | | | | | | | |
|---|---|---|---|---|---|---|---|---|
| Group | animals | Vehicle | formulation | concentration | Dose per mouse | | Volume | |
| 1 | 6 | saline | 0 | 0 | 0 | | 4 × 20 | μL (i.d.) |
| 2 | 6 | / | Adapalene gel 0.1% | 0 | 100 | μg | 100 | μL (topic) |
| 3 | 6 | saline | Dysport dose 1 | 1.25 U/mL | 0.1 | U | 4 × 20 | μL (i.d.) |
| 4 | 6 | saline | Dysport dose 2 | 3.75 U/mL | 0.3 | U | 4 × 20 | μL (i.d.) |
| 5 | 6 | saline | Dysport dose 3 | 12.5 U/mL | 1 | U | 4 × 20 | μL (i.d.) |

6 animals per group were administered (30 animals to be administered) on day 1.

### Intradermal Injection (BoNT) and Application of the Reference Compound (Adapalene)

Intradermal injection (groups 1, 3, 4 and 5) was performed (on day 1 only) under anaesthesia (isoflurane) into 4 sites on the back skin (4×20 μL into a rectangle area about 2-3 cm², 80 μL per mouse, see FIG. 1) using 30 G needles connected to a 100 μL micro-syringe.

The topical application (anaesthesia unnecessary) of the reference compound (group 2) was done daily on a back area about 4 cm², which corresponds to a rectangle about 1.5 cm to 2.5 cm on the skin. After the application of the gel (100 μL), the area was smoothly massaged with a gloved finger for 10 seconds.

### Efficacy Endpoints

On 4 occasions, on days 1 (before treatment), 5, 10 and 15 (before euthanasia), the following parameters were evaluated:
Dorsal skin thickness using caliper on the application site
Macroscopic description of the skin on the application site (if applicable)

Additional observations such as erythema or scaling were noted when evidenced. These parameters were scored as indicated below:

| score | erythema | scaling |
|---|---|---|
| 0 | Normal | Normal |
| 1 | Slight | Slight |
| 2 | Moderate | Moderate |
| 3 | Important | Important |

Calibrated digital pictures of the area of application of the formulations were taken on 4 occasions (days 1, 5, 10 and 15) before the efficacy endpoint are recorded.

### Collection and Processing of the Samples

On day 15 (after injection/application), mice were euthanized by cervical dislocation under isoflurane anaesthesia. Back skin samples were removed at the application site, one part of the tissue was identified for histological analyses, transferred in formalin for 48 h then in EtOH. The other part of the samples was frozen and stored at −80° C. for ELISA assay.

For lipid analysis (performed on day 15), a glass cone was applied on the dorsal skin area for about 10 seconds and removed to collect the sebum and kept at −80° C. until analysis.

### Histology

Histology was processed by ANEXPLO platform, Toulouse, France. After paraffin inclusion, slices were performed and HES (Haematoxylin/Eosin/Safran, for structural/anatomical analysis of e.g. utricule size) and Ki67 (proliferation marker) (immunohistochemistry) staining were done on 2 different slices. Stained slices were digitalized (microscope objective ×20) and archived.

Image scanning, image analysis and anatomo-pathological evaluation were performed with blinded data to permit the non-biased analysis of the following parameters (via classical surface measurement/nuclei counting using a histology software):
Utriculi surface measurements (HES)
Sebaceous glands surface (HES)
Epidermis thickness (HES)
Inflammation in dermis (HES)
Keratinocytes and fibroblasts proliferation (Ki67 only)

### Lipidomics Analysis

Sebum samples (n=30) were analysed for lipidomics to evaluate sebum lipids composition using gas chromatography analysis after lipid extraction.

The following lipid families were analysed:
Free fatty acids/monoglycerides
Squalene
Cholesterol
Wax esters/Diglycerides
Cholesteryl esters/Triglycerides

Lipid extraction was performed as followed:
2 ml of n-hexane were added on the glass cone (harbouring the collected sebum) in a 5 ml glass tube
Tubes were vortexed 1 min
Solvent was separated and placed in another clean glass tube weighed prior to analysis
Evaporation of solvent under a stream of nitrogen at +60° C.
Tubes were weighed for total sebum collection calculation (the weighing scale having been appropriately tarred/balanced)
700 μL of chloroform/isopropanol 1:1 was added to weighed dry tubes
Tubes were vortexed 1 min
300 μL of water was added
Tubes were vortexed 1 min and placed on orbital shaker for 10 min
Tubes were centrifuged 5 min at 3000 g at ambient temperature
Organic phase was separated and placed in a clean glass tube
Evaporation of solvent under a stream of nitrogen at +60° C.
Dry residue was extracted with 100 μL of ethyl acetate and placed in GC vial for injection and kept at +4° C. until analysis

GC analysis was performed on a GC/FID apparatus 7890B (Agilent), and acquisition was performed with Empower 3, ver. 3471 (Waters).

A capillary GC column was used Zebron ZB-SHT, ref.7HG-G015-02, dimensions 30 m×0.25 mm×0.1 μm. The following GC method was used:
Injector temperature 350° C./Split mode injection
Column temperature gradient:
   80° C. to 240° C. at 10° C./min
   240° C. to 320° C. at 5° C./min
   320° C. to 350° C. at 2° C./min
   350° C. for 20 min
FID detector temperature 250° C.
FID Hydrogen flow 35 ml/min
FID Air flow 350 ml/min
FID Make up Helium flow 25 ml/min

After acquisition, mean chromatograms were calculated per group and compared between groups. Peaks of interest were integrated and used for comparison between groups. In more detail, the integration of the (main) peak of the chromatogram corresponding to the lipid of interest was achieved, and the peak area of each compound (lipid) was determined according to the peak area of the ‘standards’ (known concentration) of the relevant lipids.

### ELISA Assay

Frozen skin samples (n=30) were analysed using a commercial kit for substance P (Enzo, Substance P ELISA Kit, France) and IL-1 alpha (Mabtech AB, mouse IL-1 alpha ELISA development kit, France). ELISA assays were done in duplicate on tissue extracts obtained from skin samples, as indicated by the manufacturer.

Tissue extracts were obtained after mechanical grinding (Precellys®, 3 cycles, 6800 rpm, 30s with beads CKMixSO-R) in DPBS buffer supplemented with a protease inhibitor cocktail (Roche, Complete, Mini Easy Pack, Protease Inhibitor Cocktail Tablets, France). After grinding, extract were centrifuged at 12000×g for 10 min at 4° C. Supernatants were removed and an additional centrifugation was performed at 12000×g for 10 min at 4° C. Supernatants were stored at −80° C. Before grinding, samples were weighed and tissue dilution in the buffer was applied identically in all samples (10 mg tissue in 0.1 ml buffer). ELISA assay for Substance P was performed on a 96 wells plate. The analysis was performed according to the method given by the supplier and according to the procedure below:

50 μL of tissue extract or standards or buffer (blank) were added into the appropriate wells with 50 μL of buffer, 50 μL of blue Conjugate and 50 μL of Antibody and incubated at room temperature for 2 hours at 500 rpm. The plate was then empty and washed 3 times with the wash solution. After the final wash, total remaining wash buffer was removed and 200 μL of p-Npp Substrate solution was added to every wells and incubated 1 hour at room temperature. Then, 50 μL of stop Solution was added in each well and absorbance was read at 405 nm. Single calibration standard curve was analysed the day of analysis. Blank subtraction and quantification from standard were performed according to the supplier recommendations.

Elisa Assay for IL-1 alpha was performed on a 96 wells plate. The analysis was performed according to the method given by the supplier and according to the procedure below:
mAb against IL-1 alpha was coated into the wells at a concentration of 2 μg/ml per well over night at 4° C.
Wash 2 times with PBS (200 μL/well)
Add 200 μL/well PBST/BSA and incubate for 1 hour at room temperature
Wash the plate 5 times with PBST (200 μL/well)
Add 100 μL tissue extract (dilution 1: 1000 in PBST/BSA) or standards or blank into the appropriate wells and incubate for 2 hours at room temperature
Wash 5 times with PBST (200 μL/well)
Add mAb biotinylated (100 μL/well) at the final concentration 0.5 μg/ml and incubate for 1 hour at room temperature
Wash the plate 5 times with PBST (200 μL/well)
Add Streptavidin-ALP (100 μL/well) and incubate for 1 hour at room temperature
Wash the plate 5 times with PBST (200 μL/well)
Add p-Npp Substrate Solution (100 μL/well)
Measure the optical density at 405 nm after the appearance of the yellow coloration

Single calibration standard curve was analysed the day of analysis.

Concentration of substance P and IL-1 alpha are expressed relative to tissue weight (of the sample).

### Example 1

### Intradermal Administration of BoNT/A Did not Alter Sebum Levels on the Epidermis

Sebum levels on the epidermis of mice of Groups 1-5 (as described under the ‘Treatment’ section of the Materials and Methods above) were measured.

A glass cone was applied on the dorsal skin area for about 10 seconds and removed to collect the sebum, and then weighed on a fine scale (the scale was balanced/tared with the same glass cone prior to contacting it with the skin to collect sebum). The resulting weight measurement corresponded to the weight of sebum collected (thus providing a readout of the level of sebum on the skin of the mice).

There was no statistically significant difference in the level of sebum between Group 1 (treated with vehicle only) and any one of the BoNT/A treated groups (Groups 3-5) (see FIG. 1). The only group showing a change (e.g. decrease) in sebum levels was Group 2 (the adapalene treated group).

FIG. 1 demonstrates the mean (and standard deviation) of six samples.

### Example 2

BoNT/A induces increased levels of fatty acid in the sebum of the epidermis Peak areas (e.g. chromatogram peak areas) of a fatty acid of the sebaceous gland of interest in sebum samples are outlined in the Table below. Intradermal administration of BoNT/A induced increased excretion of fatty acid to the epidermis (in levels closely following the dose), but Adapalene did not. This data is illustrated in a bar chart in FIG. 2:

| group 1 - | | group 2 - | | group 3 - | | group 4 - | | group 5 - | |
|---|---|---|---|---|---|---|---|---|---|
| vehicle | | Adapalene | | Dysport 0.1 U | | Dysport 0.3 U | | Dysport 1 U | |
| Animal | Peak area | Animal | Peak area | Animal | Peak area | Animal | Peak area | Animal | Peak area |
| ID | (μV*sec) | ID | (μV*sec) | ID | (μV*sec) | ID | (μV*sec) | ID | (μV*sec) |
| 1 | 3.069 | 7 | 4.375 | 13 | 14.713 | 19 | 15.603 | 25 | 22.681 |
| 2 | 8.288 | 8 | 7.074 | 14 | 10.373 | 20 | 12.281 | 26 | 14.233 |
| 3 | 6.749 | 9 | 4.869 | 15 | 9.166 | 21 | 10.479 | 27 | 9.245 |
| 4 | 10.604 | 10 | 13.608 | 16 | 11.775 | 22 | 12.132 | 28 | 21.457 |
| 5 | 8.850 | 11 | 6.204 | 17 | 14.899 | 23 | 13.981 | 29 | 10.943 |
| 6 | 13.239 | 12 | 7.882 | 18 | 15.801 | 24 | 12.156 | 30 | 14.846 |
| Mean | 8.5 | Mean | 7.3 | Mean | 12.8 | Mean | 12.8 | Mean | 15.6 |
| SD | 3.5 | SD | 3.3 | SD | 2.7 | SD | 1.8 | SD | 5.5 |

### Example 3

### BoNT/A Induces Increased Levels of Cholesterol in the Sebum of the Epidermis

Peak areas of cholesterol (of the sebaceous gland) in sebum samples are outlined in the Table below. Intradermal administration of BoNT/A induced increased excretion of cholesterol to the epidermis (in levels closely following the dose). This data is illustrated in a bar chart in FIG. 4:

| group 1 - | | group 2 - | | group 3 - | | group 4 - | | group 5 - | |
|---|---|---|---|---|---|---|---|---|---|
| vehicle | | Adapalene | | Dysport 0.1 U | | Dysport 0.3 U | | Dysport 1 U | |
| Animal | Peak area | Animal | Peak area | Animal | Peak area | Animal | Peak area | Animal | Peak area |
| ID | (μV*sec) | ID | (μV*sec) | ID | (μV*sec) | ID | (μV*sec) | ID | (μV*sec) |
| 1 | 11.036 | 7 | 38.921 | 13 | 79.338 | 19 | 101.645 | 25 | 152.057 |
| 2 | 26.101 | 8 | 59.612 | 14 | 62.687 | 20 | 77.480 | 26 | 73.678 |
| 3 | 32.789 | 9 | 43.932 | 15 | 58.712 | 21 | 66.999 | 27 | 63.521 |
| 4 | 57.242 | 10 | 114.258 | 16 | 68.635 | 22 | 83.102 | 28 | 152.254 |
| 5 | 51.177 | 11 | 53.274 | 17 | 93.606 | 23 | 82.409 | 29 | 78.120 |
| 6 | 66.478 | 12 | 69.339 | 18 | 91.404 | 24 | 75.833 | 30 | 107.209 |
| Mean | 40.8 | Mean | 63.2 | Mean | 75.7 | Mean | 81.2 | Mean | 104.5 |
| SD | 21.0 | SD | 27.3 | SD | 14.8 | SD | 11.6 | SD | 39.7 |

### Example 4

### BoNT/A Induces Increased Levels of Squalene in the Sebum of the Epidermis

Peak areas of squalene (of the sebaceous gland) in sebum samples are outlined in the Table below. Intradermal administration of BoNT/A induced increased excretion of squalene to the epidermis (in levels closely following the dose). This was particularly surprising, as the sebum of mice generally does not comprise (or comprises only an insignificant amount of) squalene. This data is illustrated in a bar chart in FIG. 3:

| group 1 - | | group 2 - | | group 3 - | | group 4 - | | group 5 - | |
|---|---|---|---|---|---|---|---|---|---|
| vehicle | | Adapalene | | Dysport 0.1 U | | Dysport 0.3 U | | Dysport 1 U | |
| Animal | Peak area | Animal | Peak area | Animal | Peak area | Animal | Peak area | Animal | Peak area |
| ID | (μV*sec) | ID | (μV*sec) | ID | (μV*sec) | ID | (μV*sec) | ID | (μV*sec) |
| 1 | 1.201 | 7 | 3.886 | 13 | 8.814 | 19 | 11.025 | 25 | 18.622 |
| 2 | 3.280 | 8 | 5.974 | 14 | 8.681 | 20 | 8.798 | 26 | 11.864 |
| 3 | 3.948 | 9 | 3.441 | 15 | 8.901 | 21 | 8.851 | 27 | 7.259 |
| 4 | 6.591 | 10 | 13.604 | 16 | 7.720 | 22 | 10.636 | 28 | 18.808 |
| 5 | 5.722 | 11 | 6.037 | 17 | 9.253 | 23 | 10.929 | 29 | 10.667 |
| 6 | 8.076 | 12 | 7.183 | 18 | 11.699 | 24 | 10.433 | 30 | 12.060 |
| Mean | 4.8 | Mean | 6.7 | Mean | 9.2 | Mean | 10.1 | Mean | 13.2 |
| SD | 2.5 | SD | 3.7 | SD | 1.3 | SD | 1.0 | SD | 4.6 |

### Example 5

### BoNT/A Induces Increased Excretion of Squalene to the Epidermis

Peak areas of wax esters (of the sebaceous gland) in sebum samples are outlined in the Table below. Intradermal administration of BoNT/A induced increased excretion of wax esters to the epidermis (in levels closely following the dose). Three wax esters (refereed to as Wax 2, 3, 4) were analysed. This data is illustrated in a bar chart in FIG. 5:

### Wax 2:

| group 1 - | | group 2 - | | group 3 - | | group 4 - | | group 5 - | |
|---|---|---|---|---|---|---|---|---|---|
| vehicle | | Adapalene | | Dysport 0.1 U | | Dysport 0.3 U | | Dysport 1 U | |
| Animal | Peak area | Animal | Peak area | Animal | Peak area | Animal | Peak area | Animal | Peak area |
| ID | (μV*sec) | ID | (μV*sec) | ID | (μV*sec) | ID | (μV*sec) | ID | (μV*sec) |
| 1 | 0.455 | 7 | 1.919 | 13 | 2.779 | 19 | 2.296 | 25 | 5.025 |
| 2 | 0.979 | 8 | 1.820 | 14 | 3.705 | 20 | 1.883 | 26 | 5.468 |
| 3 | 1.587 | 9 | 0.952 | 15 | 4.260 | 21 | 3.428 | 27 | 1.874 |
| 4 | 1.899 | 10 | 3.634 | 16 | 2.267 | 22 | 3.720 | 28 | 5.166 |
| 5 | 1.595 | 11 | 1.934 | 17 | 3.215 | 23 | 4.131 | 29 | 4.528 |
| 6 | 2.237 | 12 | 2.342 | 18 | 3.803 | 24 | 4.327 | 30 | 3.830 |
| Mean | 1.5 | Mean | 2.1 | Mean | 3.3 | Mean | 3.3 | Mean | 4.3 |
| SD | 0.6 | SD | 0.9 | SD | 0.7 | SD | 1.0 | SD | 1.3 |

### Wax 3:

| group 1 - | | group 2 - | | group 3 - | | group 4 - | | group 5 - | |
|---|---|---|---|---|---|---|---|---|---|
| vehicle | | Adapalene | | Dysport 0.1 U | | Dysport 0.3 U | | Dysport 1 U | |
| Animal | Peak area | Animal | Peak area | Animal | Peak area | Animal | Peak area | Animal | Peak area |
| ID | (μV*sec) | ID | (μV*sec) | ID | (μV*sec) | ID | (μV*sec) | ID | (μV*sec) |
| 1 | 0.194 | 7 | 0.707 | 13 | 2.013 | 19 | 1.834 | 25 | 4.363 |
| 2 | 0.567 | 8 | 0.947 | 14 | 1.896 | 20 | 2.108 | 26 | 2.257 |
| 3 | 0.731 | 9 | 0.774 | 15 | 1.872 | 21 | 1.814 | 27 | 2.401 |
| 4 | 1.331 | 10 | 1.735 | 16 | 1.997 | 22 | 1.959 | 28 | 5.776 |
| 5 | 1.113 | 11 | 0.909 | 17 | 2.488 | 23 | 2.532 | 29 | 3.032 |
| 6 | 1.877 | 12 | 1.161 | 18 | 2.820 | 24 | 2.305 | 30 | 2.899 |
| Mean | 0.97 | Mean | 1.04 | Mean | 2.19 | Mean | 2.09 | Mean | 3.45 |
| SD | 0.60 | SD | 0.38 | SD | 0.39 | SD | 0.28 | SD | 1.36 |

### Wax 4:

| group 1 - | | group 2 - | | group 3 - | | group 4 - | | group 5 - | |
|---|---|---|---|---|---|---|---|---|---|
| vehicle | | Adapalene | | Dysport 0.1 U | | Dysport 0.3 U | | Dysport 1 U | |
| Animal | Peak area | Animal | Peak area | Animal | Peak area | Animal | Peak area | Animal | Peak area |
| ID | (μV*sec) | ID | (μV*sec) | ID | (μV*sec) | ID | (μV*sec) | ID | (μV*sec) |
| 1 | 0.239 | 7 | 0.860 | 13 | 3.369 | 19 | 2.886 | 25 | 9.703 |
| 2 | 0.674 | 8 | 0.938 | 14 | 3.838 | 20 | 4.150 | 26 | 2.968 |
| 3 | 1.263 | 9 | 0.692 | 15 | 3.707 | 21 | 2.849 | 27 | 6.110 |
| 4 | 2.512 | 10 | 2.067 | 16 | 4.589 | 22 | 3.139 | 28 | 13.906 |
| 5 | 1.801 | 11 | 0.850 | 17 | 5.439 | 23 | 4.827 | 29 | 6.596 |
| 6 | 4.034 | 12 | 1.287 | 18 | 5.719 | 24 | 4.245 | 30 | 5.844 |
| Mean | 1.8 | Mean | 1.1 | Mean | 4.4 | Mean | 3.7 | Mean | 7.5 |
| SD | 1.4 | SD | 0.5 | SD | 1.0 | SD | 0.8 | SD | 3.8 |

### Example 6

### BoNT/A Induces Increased Levels of Cholesteryl Ester in the Sebum of the Epidermis

Peak areas of cholesteryl ester (of the sebaceous gland) in sebum samples are outlined in the Table below. Intradermal administration of BoNT/A induced increased excretion of cholesteryl ester to the epidermis (in levels closely following the dose). This data is illustrated in a bar chart in FIG. 6:

| group 1 - | | group 2 - | | group 3 - | | group 4 - | | group 5 - | |
|---|---|---|---|---|---|---|---|---|---|
| vehicle | | Adapalene | | Dysport 0.1 U | | Dysport 0.3 U | | Dysport 1 U | |
| Animal | Peak area | Animal | Peak area | Animal | Peak area | Animal | Peak area | Animal | Peak area |
| ID | (μV*sec) | ID | (μV*sec) | ID | (μV*sec) | ID | (μV*sec) | ID | (μV*sec) |
| 1 | ND | 7 | 0.426 | 13 | 3.311 | 19 | 3.403 | 25 | 8.395 |
| 2 | 0.424 | 8 | 1.216 | 14 | 3.551 | 20 | 3.727 | 26 | 2.899 |
| 3 | 0.681 | 9 | 1.028 | 15 | 3.666 | 21 | 3.033 | 27 | 6.178 |
| 4 | 1.718 | 10 | 4.900 | 16 | 3.774 | 22 | 3.378 | 28 | 13.798 |
| 5 | 1.394 | 11 | 1.822 | 17 | 4.930 | 23 | 4.941 | 29 | 6.567 |
| 6 | 2.376 | 12 | 1.508 | 18 | 5.280 | 24 | 4.569 | 30 | 5.091 |
| Mean | 1.3 | Mean | 1.8 | Mean | 4.1 | Mean | 3.8 | Mean | 7.2 |
| SD | 0.8 | SD | 1.6 | SD | 0.8 | SD | 0.8 | SD | 3.8 |

### Example 7

### BoNT/A Induces Retention of Substance P in the Dermis

Skin tissues were homogenized in DPBS containing a protease inhibitor cocktail (1 mg tissue: 0.01 ml buffer) by a mechanical grinding method using ceramic beads (3 cycles 6800 rpm, 30 second), then centrifuged at 12000×g for 10 min at 4° C. Supernatants were collected and Substance P levels were estimated using ELISA kits (as explained in Materials & Methods). Substance P is expressed as μg/g tissue.

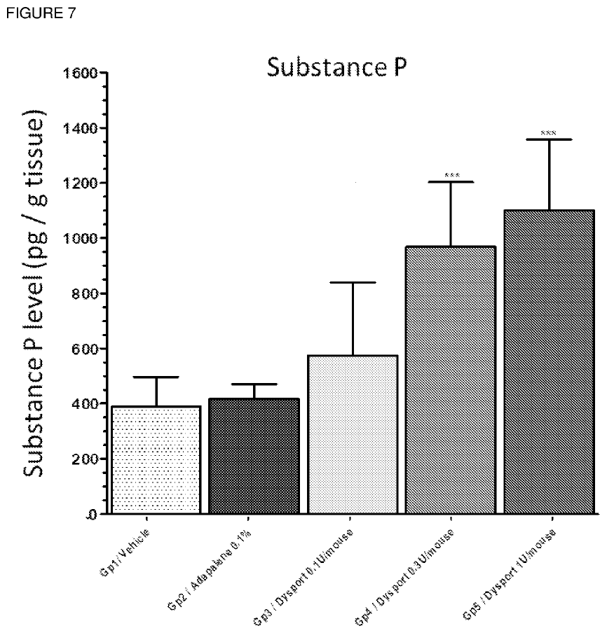

Intradermal administration of BoNT/A induced retention of Substance P in the epidermis (in levels closely following the dose). Levels of Substance P in back samples are shown in the Table below. This data is illustrated in a bar chart in FIG. 7:

| group 1— | | group 2— | | group 3— | |
|---|---|---|---|---|---|
| vehicle | | Adapalene | | Dysport 0.1 U | |
| | Concen- | | Concen- | | Concen- |
| | tration | | tration | | tration |
| Animal | (pg/g | Animal | (pg/g | Animal | (pg/g |
| ID | tissue) | ID | tissue) | ID | tissue) |
| 1 | 469.6 | 7 | 409.8 | 13 | 550.0 |
| 2 | 323.6 | 8 | 403.9 | 14 | 940.9 |
| 3 | 323.8 | 9 | 491.3 | 15 | 207.8 |
| 4 | 390.9 | 10 | 335.5 | 16 | 808.2 |
| 5 | 275.4 | 11 | 414.1 | 17 | 431.6 |
| 6 | 558.0 | 12 | 453.4 | 18 | 513.5 |
| Mean | 390.2 | Mean | 418.0 | Mean | 575.3 |
| SD | 106.3 | SD | 52.37 | SD | 263.8 |

| group 4—Dysport 0.3 U | | Group 5—Dysport 1 U | |
|---|---|---|---|
| | Concen- | | Concen- |
| | tration | | tration |
| Animal | (pg/g | Animal | (pg/g |
| ID | tissue) | ID | tissue) |
| 19 | 1231.7 | 25 | 998.6 |
| 20 | 814.2 | 26 | 1137.8 |
| 21 | 1044.3 | 27 | 1004.9 |
| 22 | 618.5 | 28 | 721.1 |
| 23 | 918.9 | 29 | 1444.2 |
| 24 | 1187.8 | 30 | 1304.0 |
| Mean | 969.2 | Mean | 1101.8 |
| SD | 233.2 | SD | 254.7 |

### Example 8

Evolution of the Clinical Scores of Erythema and Scaling On days 1, 5, 10 and 15, clinical scores (erythema and scaling on back skin) were tracked and recorded. In this study, no erythema and scaling was observed for groups 1, 3 and 4 (Vehicle, Dysport 0.1 U and Dysport 0.3 U, respectively).

Higher erythema and scaling scores were observed in the reference group 2 compared with the Dysport treated groups. Only 2 mice in the group 5 treated by Dysport 1 U exhibited a very slight scaling mark on day 5 (score=1), which disappeared on day 10.

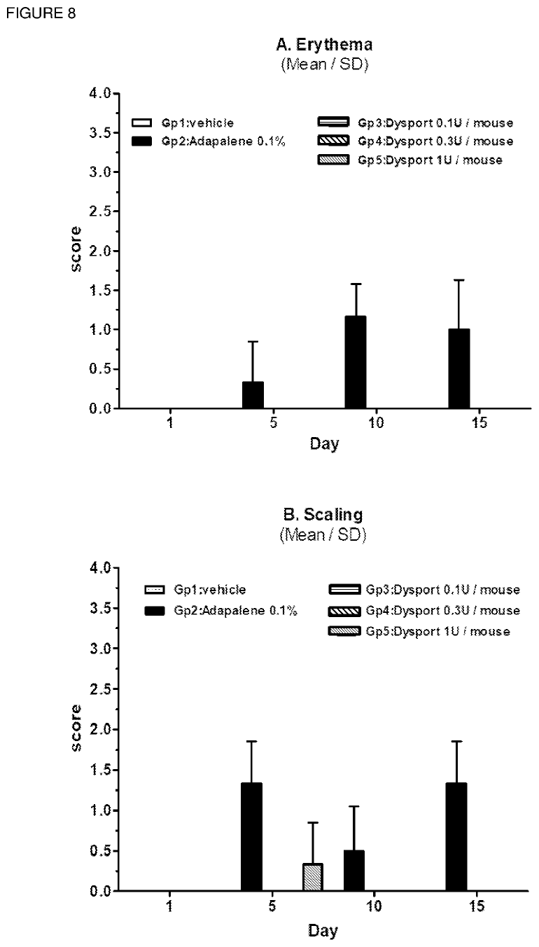

Scores are outlined in the Tables below. This data is illustrated in bar charts in FIG. 8:

Evolution of the clinical scores (erythema) on the back skin in group 2 (Adapalene) (mean+/−SD):

Group 2 - Adapalene 0.1%

| | | Animal ID | | | | | | | |
|---|---|---|---|---|---|---|---|---|---|
| Parameter | Day | 7 | 8 | 9 | 10 | 11 | 12 | Mean | SD |
| Erythema | 1 | 0 | 0 | 0 | 0 | 0 | 0 | 0 | 0 |
| score | 5 | 0 | 0 | 0 | 0 | 1 | 1 | 0.3 | 0.5 |
| | 10 | 1 | 1 | 2 | 1 | 1 | 1 | 1.2 | 0.4 |
| | 15 | 1 | 1 | 1 | 2 | 1 | 0 | 1.0 | 0.6 |

Evolution of the clinical scores (scaling) on the back skin in groups 2 (Adapalene) and 5 (Dysport 1 U/mouse) (mean+/−SD):

Group 2 - Adapalene

| | | Animal ID | | | | | | | |
|---|---|---|---|---|---|---|---|---|---|
| Parameter | Day | 7 | 8 | 9 | 10 | 11 | 12 | Mean | SD |
| Scaling | 1 | 0 | 0 | 0 | 0 | 0 | 0 | 0 | 0 |
| score | 5 | 1 | 1 | 2 | 1 | 1 | 2 | 1.3 | 0.5 |
| | 10 | 0 | 0 | 0 | 1 | 1 | 1 | 0.5 | 0.5 |
| | 15 | 1 | 2 | 1 | 2 | 1 | 1 | 1.3 | 0.5 |

Group 5 - Dysport 1 U/mouse

| | | Animal ID | | | | | | | |
|---|---|---|---|---|---|---|---|---|---|
| Parameter | Day | 25 | 26 | 27 | 28 | 29 | 30 | Mean | SD |
| Scaling | 1 | 0 | 0 | 0 | 0 | 0 | 0 | 0 | 0 |
| score | 5 | 1 | 0 | 1 | 0 | 0 | 0 | 0.3 | 0.5 |
| | 10 | 0 | 0 | 0 | 0 | 0 | 0 | 0 | 0 |
| | 15 | 0 | 0 | 0 | 0 | 0 | 0 | 0 | 0 |

### Example 9

Sebaceous Glands Surface following Treatment

On day 15, skin sample collected around the two upper injection sites were collected and fixed for HES (Haematoxylin/Eosin/Safran) and Ki67 staining.

Several parameters were assessed on HES slices: epidermis thickness, dermis inflammation, utricles and sebaceous glands surface.

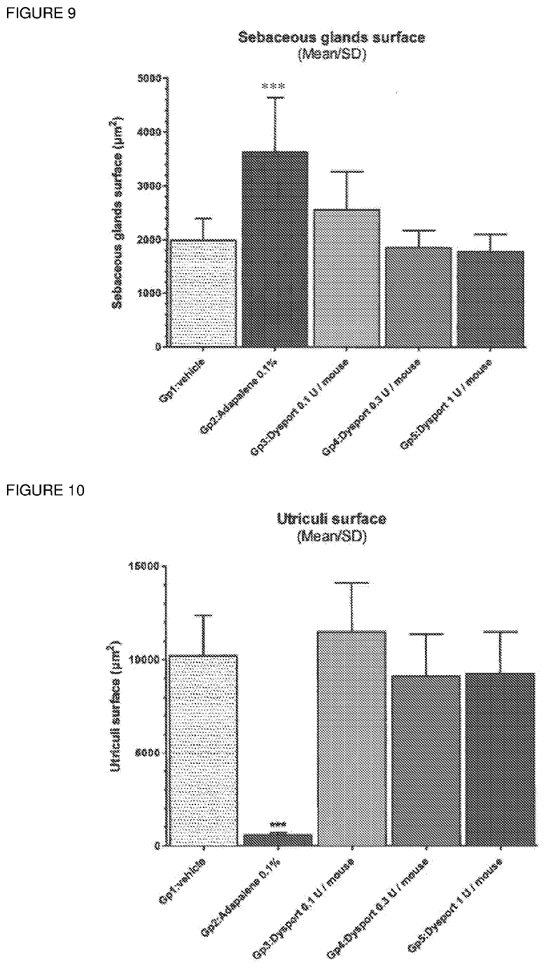

The surface of 10 sebaceous glands was calculated for each mouse on HES slices. Adapalene significantly increased sebaceous glands surface, but Dysport (BoNT/A) did not. Results (mean of 10 measurements per mouse) with SD are shown in the Table below. This data is illustrated in bar charts in FIG. 9:

| group 1— | | group 2— | | group 3— | |
|---|---|---|---|---|---|
| vehicle | | Adapalene | | Dysport 0.1 U | |
| | Seba- | | Seba- | | Seba- |
| | ceous | | ceous | | ceous |
| | gland | | gland | | gland |
| Animal | surface | Animal | surface | Animal | surface |
| ID | (μm²) | ID | (μm²) | ID | (μm²) |
| 1 | 1684.7 | 7 | 3242.0 | 13 | 2072.3 |
| 2 | 2152.9 | 8 | 4040.2 | 14 | 2520.6 |
| 3 | 1825.4 | 9 | 2344.5 | 15 | 3293.6 |
| 4 | 2072.0 | 10 | 3534.4 | 16 | 2093.7 |
| 5 | 2682.4 | 11 | 5356.4 | 17 | 3548.7 |
| 6 | 1538.9 | 12 | 3301.4 | 18 | 1826.3 |
| Mean | 1992.7 | Mean | 3636.5 | Mean | 2559.2 |
| SD | 409.0 | SD | 1006.8 | SD | 708.6 |

| group 4—Dysport 0.3 U | | Group 5—Dysport 1 U | |
|---|---|---|---|
| | Sebaceous | | Sebaceous |
| Animal | gland | Animal | gland |
| ID | surface (μm²) | ID | surface (μm²) |
| 19 | 1624.2 | 25 | 1593.4 |
| 20 | 1764.6 | 26 | 2123.4 |
| 21 | 1747.9 | 27 | 2109.6 |
| 22 | 2197.0 | 28 | 1987.9 |
| 23 | 2300.1 | 29 | 1470.1 |
| 24 | 1505.2 | 30 | 1414.3 |
| Mean | 1856.5 | Mean | 1783.1 |
| SD | 319.5 | SD | 326.9 |

### Example 10

### Utriculi Surface Following Treatment

The surface of 10 utricles was calculated for each mouse on HES slices. Adapalene significantly decreased utricle's surfaces, but Dysport (BoNT/A) did not. Results (mean of 10 measurements per mouse) with SD are shown in the Table below. This data is illustrated in bar charts in FIG. 10:

| group 1— | | group 2— | | group 3— | |
|---|---|---|---|---|---|
| vehicle | | Adapalene | | Dysport 0.1 U | |
| | utriculi | | utriculi | | utriculi |
| Animal | surface | Animal | surface | Animal | surface |
| ID | (μm²) | ID | (μm²) | ID | (μm²) |
| 1 | 8816.8 | 7 | 515.2 | 13 | 11871.1 |
| 2 | 8272.8 | 8 | 806.1 | 14 | 8247.5 |
| 3 | 8257.6 | 9 | 527.4 | 15 | 12172.1 |
| 4 | 11590.3 | 10 | 605.0 | 16 | 8537.3 |
| 5 | 10643.4 | 11 | 515.3 | 17 | 13359.6 |
| 6 | 13615.5 | 12 | 545.4 | 18 | 14739.6 |
| Mean | 10199.4 | Mean | 585.7 | Mean | 11487.9 |
| SD | 2153.9 | SD | 113.1 | SD | 2603.8 |

| group 4—Dysport 0.3 U | | Group 5—Dysport 1 U | |
|---|---|---|---|
| | utriculi | | utriculi |
| Animal | surface | Animal | surface |
| ID | (μm²) | ID | (μm²) |
| 19 | 5705.2 | 25 | 9614.3 |
| 20 | 7213.5 | 26 | 5024.0 |
| 21 | 10045.7 | 27 | 10840.1 |
| 22 | 10159.0 | 28 | 10875.4 |
| 23 | 9468.5 | 29 | 10513.2 |
| 24 | 11972.7 | 30 | 8495.0 |
| Mean | 9094.1 | Mean | 9227.0 |
| SD | 2257.8 | SD | 2250.5 |

### Example 11

Epidermis Thickness following Treatment

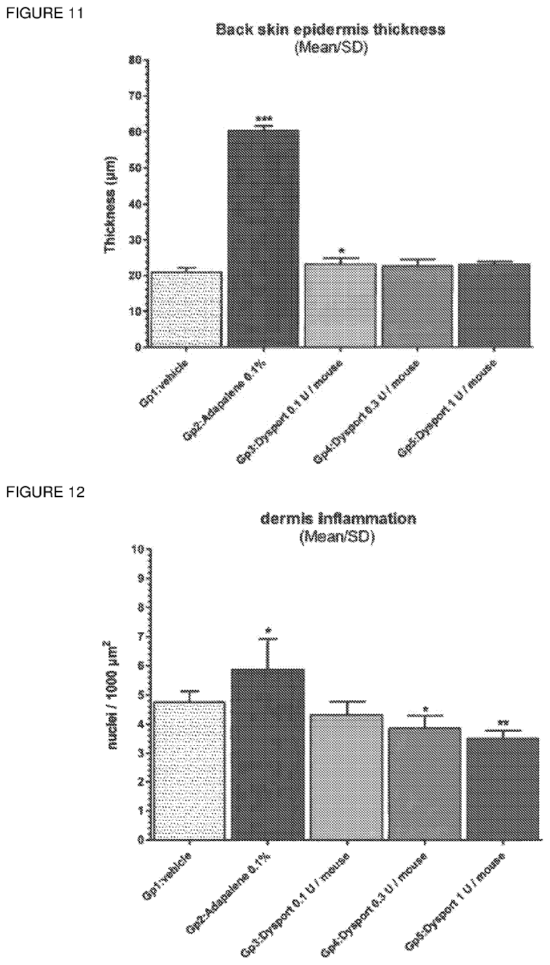

The thickness of epidermis (back skin area) was calculated for each mouse on HES slices. Adapalene significantly increased thickness of epidermis, but Dysport (BoNT/A) did not. Results (mean of 6 measurements per mouse) with SD are shown in the Table below. This data is illustrated in bar charts in FIG. 11:

| group 1— | | group 2— | | group 3— | |
|---|---|---|---|---|---|
| vehicle | | Adapalene | | Dysport 0.1 U | |
| | Epi- | | Epi- | | Epi- |
| | dermis | | dermis | | dermis |
| | thick- | | thick- | | thick- |
| Animal | ness | Animal | ness | Animal | ness |
| ID | (μm²) | ID | (μm²) | ID | (μm²) |
| 1 | 19.3 | 7 | 61.3 | 13 | 24.8 |
| 2 | 22.0 | 8 | 59.3 | 14 | 20.0 |
| 3 | 23.1 | 9 | 59.9 | 15 | 23.3 |
| 4 | 20.6 | 10 | 58.8 | 16 | 24.2 |
| 5 | 20.7 | 11 | 62.4 | 17 | 24.0 |
| 6 | 19.8 | 12 | 59.7 | 18 | 23.2 |
| Mean | 20.9 | Mean | 60.2 | Mean | 23.3 |
| SD | 1.4 | SD | 1.4 | SD | 1.7 |

| group 4—Dysport 0.3 U | | Group 5—Dysport 1 U | |
|---|---|---|---|
| | Epi- | | Epi- |
| | dermis | | dermis |
| Animal | thick- | Animal | thick- |
| ID | ness | ID | ness |
| 19 | 23.9 | 25 | 22.5 |
| 20 | 21.5 | 26 | 24.1 |
| 21 | 26.0 | 27 | 22.3 |
| 22 | 20.8 | 28 | 24.0 |
| 23 | 22.3 | 29 | 23.5 |
| 24 | 21.8 | 30 | 22.3 |
| Mean | 22.7 | Mean | 23.1 |
| SD | 1.9 | SD | 0.8 |

### Example 12

### Dermis Inflammation Following Treatment

The inflammation into the dermis (back skin area) was assessed for each mouse on HES slices, by measuring the number of nuclei/1000 μm².

Adapalene significantly increased inflammation, but Dysport (BoNT/A) significantly decreased inflammation (in a manner which closely followed the dose). Results with SD are shown in the Table below. This data is illustrated in bar charts in FIG. 12:

| group 1— | | group 2— | | group 3— | |
|---|---|---|---|---|---|
| vehicle | | Adapalene | | Dysport 0.1 U | |
| | dermis | | Dermis | | Dermis |
| | inflam- | | inflam- | | inflam- |
| | mation | | mation | | mation |
| | (nuclei/ | | (nuclei/ | | (nuclei/ |
| Animal | 1000 | Animal | 1000 | Animal | 1000 |
| ID | μmm²) | ID | μm²) | ID | μm²) |
| 1 | 4.56 | 7 | 4.15 | 13 | 4.34 |
| 2 | 4.15 | 8 | 6.46 | 14 | 4.65 |
| 3 | 5.04 | 9 | 5.16 | 15 | 4.08 |
| 4 | 5.23 | 10 | 6.79 | 16 | 4.30 |
| 5 | 4.70 | 11 | 6.91 | 17 | 4.90 |
| 6 | 4.80 | 12 | 5.65 | 18 | 3.58 |
| Mean | 4.74 | Mean | 5.85 | Mean | 4.31 |
| SD | 0.38 | SD | 1.1 | SD | 0.46 |

| group 4— | | Group 5— | |
|---|---|---|---|
| Dysport 0.3 U | | Dysport 1 U | |
| | Dermis | | Dermis |
| | inflam- | | inflam- |
| | mation | | mation |
| | (nuclei/ | | (nuclei/ |
| Animal | 1000 | Animal | 1000 |
| ID | μm²) | ID | μm²) |
| 19 | 3.76 | 25 | 3.81 |
| 20 | 3.99 | 26 | 3.06 |
| 21 | 3.26 | 27 | 3.64 |
| 22 | 3.83 | 28 | 3.49 |
| 23 | 3.61 | 29 | 3.31 |
| 24 | 4.61 | 30 | 3.68 |
| Mean | 3.84 | Mean | 3.50 |
| SD | 0.45 | SD | 0.27 |

### Example 13

### Keratinocyte Proliferation Following Treatment

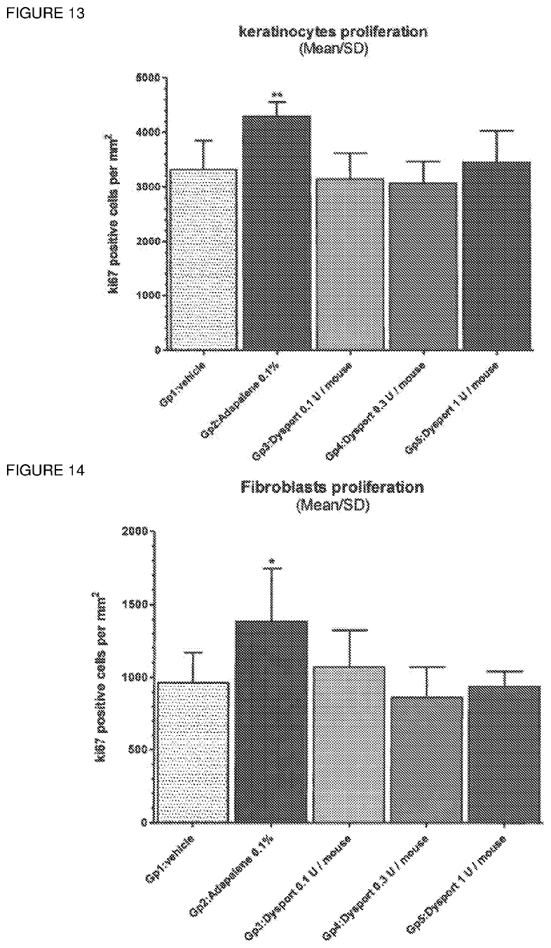

Keratinocytes proliferation was performed by automatic counting of positive Ki67 cells in epidermis from Ki67 immunostaining slices. Adapalene resulted in significantly increased keratinocyte proliferation, but Dysport (BoNT/A) did not. Results (mean of six areas per slice) are presented in the Table below. This data is illustrated in bar charts in FIG. 13:

| group 1— | | group 2— | | group 3— | |
|---|---|---|---|---|---|
| vehicle | | Adapalene | | Dysport 0.1 U | |
| | Ki67 | | Ki67 | | Ki67 |
| | positive | | positive | | positive |
| | cells | | cells | | cells |
| Animal | per | Animal | per | Animal | per |
| ID | mm² | ID | mm² | ID | mm² |
| 1 | 2956.0 | 7 | 4121.7 | 13 | 2616.4 |
| 2 | 3553.0 | 8 | 4733.8 | 14 | 3072.2 |
| 3 | 3664.2 | 9 | 4411.7 | 15 | 3277.7 |
| 4 | 3964.6 | 10 | 4260.9 | 16 | 3720.8 |
| 5 | 2481.0 | 11 | 4248.9 | 17 | 2571.8 |
| 6 | 3265.7 | 12 | 3981.7 | 18 | 3579.1 |
| Mean | 3314.4 | Mean | 4293.1 | Mean | 3139.7 |
| SD | 534 3 | SD | 259.9 | SD | 479.7 |

| group 4—Dysport 0.3 U | | Group 5—Dysport 1 U | |
|---|---|---|---|
| | Ki67 | | Ki67 |
| | positive | | positive |
| | cells | | cells |
| Animal | per | Animal | per |
| ID | mm² | ID | mm² |
| 19 | 3051.5 | 25 | 2864.2 |
| 20 | 3316.0 | 26 | 3379.2 |
| 21 | 2608.9 | 27 | 4248.0 |
| 22 | 3381.6 | 28 | 3672.1 |
| 23 | 3481.3 | 29 | 3792.6 |
| 24 | 2539.4 | 30 | 2735.6 |
| Mean | 3063.1 | Mean | 3448.6 |
| SD | 405.2 | SD | 576.5 |

### Example 14

### Fibroblast Proliferation Following Treatment

Fibroblast proliferation assessment was performed by automatic counting of positive Ki67 cells in dermis from Ki67 immunostaining slices, together with morphological analysis for the identification of fibroblasts (thus allowing quantification of the population of proliferating fibroblasts). Adapalene resulted in significantly increased fibroblast proliferation, but Dysport (BoNT/A) did not. Results (mean of six areas per slice) are presented in the Table below. This data is illustrated in bar charts in FIG. 14:

| group 1— | | group 2— | | group 3— | |
|---|---|---|---|---|---|
| vehicle | | Adapalene | | Dysport 0.1 U | |
| | Ki67 | | Ki67 | | Ki67 |
| | positive | | positive | | positive |
| | cells | | cells | | cells |
| Animal | per | Animal | per | Animal | per |
| ID | mm² | ID | mm² | ID | mm² |
| 1 | 872.3 | 7 | 1088.8 | 13 | 708.0 |
| 2 | 863.0 | 8 | 1181.9 | 14 | 920.1 |
| 3 | 1181.6 | 9 | 1229.8 | 15 | 1324.7 |
| 4 | 1233.8 | 10 | 1499.9 | 16 | 962.2 |
| 5 | 935.2 | 11 | 2072.0 | 17 | 1165.0 |
| 6 | 693.8 | 12 | 1218.1 | 18 | 1354.6 |
| Mean | 963.3 | Mean | 1381.8 | Mean | 1072.4 |
| SD | 206.2 | SD | 365.0 | SD | 253.0 |

| group 4—Dysport 0.3 U | | Group 5—Dysport 1 U | |
|---|---|---|---|
| | Ki67 | | Ki67 |
| | positive | | positive |
| | cells | | cells |
| Animal | per | Animal | per |
| ID | mm² | ID | mm² |
| 19 | 918.0 | 25 | 948.8 |
| 20 | 987.3 | 26 | 928.2 |
| 21 | 851.0 | 27 | 1087.2 |
| 22 | 598.0 | 28 | 1005.1 |
| 23 | 1159.5 | 29 | 878.1 |
| 24 | 672.9 | 30 | 782.1 |
| Mean | 864.5 | Mean | 938.4 |
| SD | 206.3 | SD | 104.5 |

### Example 15

Sebocyte Proliferation following Treatment

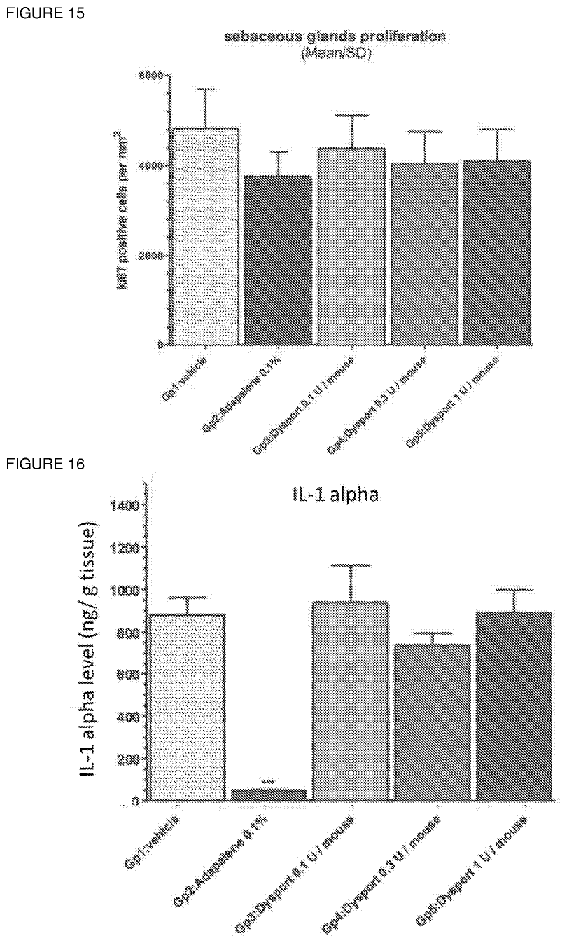

Sebocytes proliferation assessment was performed by automatic counting of positive Ki67 cells in sebaceous glands from Ki67 immunostaining slices, together with morphological analysis for the identification of sebocytes (thus allowing quantification of the population of proliferating sebocytes). No change in fibroblast proliferation was observed in any treatment group. Results (mean of six areas per slice) are presented in the Table below. This data is illustrated in bar charts in FIG. 15:

| group 1— | | group 2— | | group 3— | |
|---|---|---|---|---|---|
| vehicle | | Adapalene | | Dysport 0.1 U | |
| | Ki67 | | Ki67 | | Ki67 |
| | positive | | positive | | positive |
| | cells | | cells | | cells |
| Animal | per | Animal | per | Animal | per |
| ID | mm² | ID | mm² | ID | mm² |
| 1 | 5390.6 | 7 | 4299.5 | 13 | 4091.2 |
| 2 | 4891.2 | 8 | 2928.2 | 14 | 4307.0 |
| 3 | 5238.0 | 9 | 3722.1 | 15 | 5359.8 |
| 4 | 5637.1 | 10 | 4331.9 | 16 | 4532.2 |
| 5 | 3251.1 | 11 | 3916.9 | 17 | 3199.1 |
| 6 | 4584.8 | 12 | 3392.4 | 18 | 4849.8 |
| Mean | 4832.2 | Mean | 3765.2 | Mean | 4389.9 |
| SD | 858.8 | SD | 542.4 | SD | 733.0 |

| group 4—Dysport 0.3 U | | Group 5—Dysport 1 U | |
|---|---|---|---|
| | Ki67 | | Ki67 |
| | positive | | positive |
| | cells | | cells |
| Animal | per | Animal | per |
| ID | mm² | ID | mm² |
| 19 | 5017.9 | 25 | 3180.9 |
| 20 | 4448.9 | 26 | 3558.0 |
| 21 | 3107.3 | 27 | 5014.0 |
| 22 | 4436.8 | 28 | 4651.3 |
| 23 | 3481.3 | 29 | 4458.0 |
| 24 | 3764.3 | 30 | 3724.3 |
| Mean | 4042.8 | Mean | 4097.8 |
| SD | 712.6 | SD | 713.7 |

### Example 16

### IL-1 Alpha Cytokine Levels Following Treatment

Skin tissues were homogenized in DPBS containing a protease inhibitor cocktail (1 mg tissue: 0.01 ml buffer) by a mechanical grinding method using ceramic beads (3 cycles 6800 rpm, 30 second), then centrifuged at 12000×g for 10 min at 4° C. Supernatants were collected and IL-1 alpha levels were estimated using ELISA kits (as explained in Materials & Methods). IL-1 alpha levels are expressed as ng/g tissue.

Intradermal administration of BoNT/A did not change excretion of IL-1 alpha to the epidermis, but Aldapalene treatment significantly reduced IL-1 alpha levels. Levels of IL-1 alpha in back samples are shown in the Table below. This data is illustrated in a bar chart in FIG. 16:

| group 1— | | group 2— | | group 3— | |
|---|---|---|---|---|---|
| vehicle | | Adapalene | | Dysport 0.1 U | |
| | Con- | | Con- | | Con- |
| | cen- | | cen- | | cen- |
| | tration | | tration | | tration |
| Animal | (ng/g | Animal | (ng/g | Animal | (ng/g |
| ID | tissue) | ID | tissue) | ID | tissue) |
| 1 | 771.8 | 7 | 69.3 | 13 | 748.4 |
| 2 | 799.4 | 8 | 36.4 | 14 | 843.6 |
| 3 | 881.4 | 9 | 50.4 | 15 | 1257.4 |
| 4 | 1216.3 | 10 | 40.3 | 16 | 906.9 |
| 5 | 978.7 | 11 | 45.1 | 17 | 889.3 |
| 6 | 636.1 | 12 | 45.1 | 18 | 991.6 |
| Mean | 880.6 | Mean | 47.8 | Mean | 939.5 |
| SD | 200.3 | SD | 11.6 | SD | 175.0 |

| group 4—Dysport 0.3 U | | Group 5—Dysport 1 U | |
|---|---|---|---|
| | Con- | | Con- |
| | cen- | | cen- |
| | tration | | tration |
| Animal | (ng/g | Animal | (ng/g |
| ID | tissue) | ID | tissue) |
| 19 | 835.8 | 25 | 1047.5 |
| 20 | 571.3 | 26 | 426.7 |
| 21 | 621.5 | 27 | 1047.9 |
| 22 | 857.2 | 28 | 1150.5 |
| 23 | 649.3 | 29 | 837.8 |
| 24 | 895.9 | 30 | 841.8 |
| Mean | 738.5 | Mean | 892.1 |
| SD | 140.0 | SD | 258.7 |

### Discussion

The objective of this study was to evaluate the efficacy of Dysport in a Rhino mouse model of sebaceous glands.

Briefly, animals were injected via the intradermal route (single injection) by the neurotoxin (Dysport) at three escalating doses (0.1, 0.3 and 1 Dysport unit per mouse) under anesthesia. An additional group of mice was administered daily with Adapalene gel 0.1% by topical application for 15 days as reference. A ‘vehicle only’ group was injected via the intradermal route with saline only as a control. During this period, animals were daily observed for clinical signs relative to toxin injection. Body weight and back skin parameters were measured on days 1, 5, 10 and 15. The occurrence of erythema and scaling at the application site was also investigated on the same days. On day 15, experiment was stopped by ethical euthanasia. Sebum was collected from the treated area of skin for lipidomic assays, then the skin sample was collected to achieve histology and to determine levels of IL-1 alpha cytokine and substance P.

During the study, no major observation or adverse effect was noticed in groups 1, 2, 3 and 4. Regarding the thickness of the dorsal skin, no significant variation was evidenced in groups treated by Dysport (<10% between day 1 and day 15), a slight and non-significant thickening of the skin (+14%) was observed in the Adapalene group as compared to the vehicle group. Mice daily treated by Adapalene treated groups also exhibited more erythema on the dorsal skin (mean score between 0.3 and 1.1) from day 5 to day 15 than other groups, and scaling (mean score between 0.5 and 1.3) also increased here during the same period. No sign of erythema or scaling was observed in mice from groups 1, 3 and 4. Overall, macroscopic skin observation did not reveal any noticeable events after Dysport treatments.

Regarding histopathology on HES slices, Dysport did not significantly affect the surface area of the utricles at any of the doses tested (12% increase in the surface area of the utriculi in the group 3 (Dysport 0.1 U); 11% decrease in the group 4 (Dysport 0.3 U); 9.5% decrease in the group 5 (Dysport 1 U)), whereas Adapalene drastically inhibited this parameter (94% decrease), a major effect of retinoid activity in the Rhino acne model. As for utriculi parameter, the surface area of the sebaceous glands was not modified by Dysport, at any of the three doses tested. Mice daily treated by Adapalene exhibited a significant increase of the sebaceous glands surface (+82% versus vehicle group). This result could be explained by the opening of the utricles, which was extremely tight after Adapalene treatment, which could lead to ineffective sebum emptying and swelling of adjacent sebaceous glands. HES staining revealed a significant epidermis hyperplasia (+188% increase in epidermis thickness) associated with parakeratosis in mice treated by Adapalene. This effect is widely described during repeated treatment by retinoids as a side effect and is often associated with increase inflammation of the dermis (+23% increase in Adapalene group in this study). Dysport did not induce relevant hyperplasia of the epidermis at any of the three doses (epidermis thickness variation between 8% and 11% in groups 3, 4 and 5). Moreover, Dysport dose-dependently decreased immune cells infiltration in the dermis, evidencing a significant anti-inflammatory effect (10%, 19% and 26% decrease versus vehicle, groups 3, 4 and 5, respectively).

Finally, Ki67 immunostaining, a biomarker for cell proliferation, was used to highlight a proliferative effect of Dysport or Adapalene treatments on sebocytes (sebaceous gland cells), fibroblasts (dermis cells) and keratinocytes (epidermis cells). Dysport did not induce any proliferative effect on sebaceous glands, dermis or epidermis cells, whereas a significant increase of Ki67 positive cells was observed in both epidermis and dermis in the Adapalene group (30% and 43% increase, keratinocytes and fibroblasts, respectively). This proliferative effect was in accordance with the epidermis hyperplasia and the immune cells infiltration observed in this group. Substance P and IL-1 alpha were detected by ELISA in tissue extracts from different groups. In control and Adapalene mice, the Substance P levels were similar (390.2±106.3 and 418.0±52.4 μg/g tissue, respectively). Dysport treatment dose-dependently increased the levels of Substance P (575.3±263.8; 969.2±233.2 and 1101.8±254.7 μg/g tissue, groups 3, 4 and 5 respectively). Dysport-treated mice at the two doses 0.3 U and 1 U showed a significant change in the content of Substance P when compared to control mice (+148% and +182%, p<0.001 respectively). This result could be explained by the mechanism of action of the neurotoxin, which blocks exocytosis at the cellular level, thus leading to an accumulation of neuropeptides, as Substance P, in the nerve endings of the skin. In this way, this result validates Dysport in vivo activity.

Regarding the levels of IL-1 alpha, Adapalene repeated application drastically decreased IL-1 alpha levels (880.6±200.3 versus 47.7±11.5 ng/g tissue, p<0.001, 96% decrease). IL-1 alpha is a major cytokine in epidermis which is constitutively secreted by keratinocytes and significantly increased in a pro-inflammatory context, when keratinocytes are activated under skin environment perturbations, like skin lesion, bacterial infections or chemicals for example. The inhibition of IL-1 alpha in the Adapalene group is in accordance with the anti-inflammatory properties of the retinoid. Dysport did not change the level of IL-1 alpha at any of the three doses tested (939.5±175.0; 738.5±140.0 and 892.0±259.7 ng/g tissue in groups 3, 4 and 5 respectively).

Finally, the lipid content of the sebum from the dorsal skin was modified under Dysport treatment: as mentioned in results, by the change in the peak area of lipid compounds on chromatograms, the fatty acid levels was increased (+83%) in mice treated by Dysport 1 U. Cholesterol and squalene were also significantly increased in sebum from Dysport 0.3 U and 1 U treatments (cholesterol, +99% and +154% increase in mice treated by 0.3 U and 1 U, respectively; squalene, +110% and +175% increase in mice treated by 0.3 U and 1 U, respectively). Levels of wax esters of interest were changed under Dysport: Wax 2 level was increased by +126%, +128% and +195% in mice treated by Dysport 0.1 U, 0.3 U and 1 U, respectively. Dysport treatments also increased Wax 3 and Wax 0.4 compounds in sebum in a similar way to Wax 2. Then, cholesteryl esters in sebum were significantly increased by 440% at the highest dose of Dysport. Conversely, Adapalene did not change significantly the lipid content of sebum. Altogether, these results indicate that Dysport treatment could act on sebum content by increasing some lipid levels exhibiting protective properties on skin.

In conclusion, Adapalene evidenced a drastic reduction of the utricles surface in this study, the main effect commonly observed by retinoids in the Rhino model of acne, indicating the positive effect of this reference compound. Dysport did not change utriculi surface area at any of the dose tested but failed to induce adverse effects commonly observed with retinoid treatment, such as epidermis hyperplasia and dermis inflammation. Dysport did not enhance cell proliferation in epidermis and dermis and exhibited anti-inflammatory effect on dermis from the dose of 0.3 U, which represents a beneficial effect in this model. The pro-inflammatory cytokine IL-1 alpha was not affected by Dysport. Conversely, Dysport dose-dependently increased the levels of Substance P in the skin, a neuropeptide present in nerve endings, supporting the in vivo activity of the neurotoxin in this study. Finally, lipid content of sebum was modified under Dysport treatments, the increase of the amount of several lipids with skin protective properties could be a beneficial effect of Dysport treatment of skin conditions.

1. A method for treating a skin condition, said method comprising administering intradermally to a patient a clostridial neurotoxin, wherein following administration the clostridial neurotoxin induces: secretion of one or more sebaceous lipid(s) to an epidermal layer of skin.

2. A clostridial neurotoxin for use in a method of treating a skin condition, said method comprising administering intradermally to a patient a clostridial neurotoxin, wherein following administration the clostridial neurotoxin induces: secretion of one or more sebaceous lipid(s) to an epidermal layer of skin.

3. Non-therapeutic use of a clostridial neurotoxin for cosmetic treatment of the skin, wherein the clostridial neurotoxin induces: secretion of one or more sebaceous lipid(s) to an epidermal layer of skin.

4. Non-therapeutic use of a clostridial neurotoxin for promoting rejuvenation of the skin, wherein the clostridial neurotoxin induces: secretion of one or more sebaceous lipid(s) to an epidermal layer of skin.

5. A method for treating a skin condition, said method comprising administering intradermally to a patient a clostridial neurotoxin, wherein following administration the clostridial neurotoxin induces:
secretion of one or more sebaceous lipid(s) selected from a squalene, a fatty acid, a cholesterol, and a wax ester to an epidermal layer of skin.

6. A clostridial neurotoxin for use in a method of treating a skin condition, said method comprising administering intradermally to a patient a clostridial neurotoxin, wherein following administration the clostridial neurotoxin induces:
secretion of one or more sebaceous lipid(s) selected from a squalene, a fatty acid, a cholesterol, and a wax ester to an epidermal layer of skin.

7. Non-therapeutic use of a clostridial neurotoxin for cosmetic treatment of the skin, wherein the clostridial neurotoxin induces:
secretion of one or more sebaceous lipid(s) selected from a squalene, a fatty acid, a cholesterol, and a wax ester to an epidermal layer of skin.

8. Non-therapeutic use of a clostridial neurotoxin for promoting rejuvenation of the skin, wherein the clostridial neurotoxin induces:
secretion of one or more sebaceous lipid(s) selected from a squalene, a fatty acid, a cholesterol, and a wax ester to an epidermal layer of skin.

9. The non-therapeutic use according to any one of claims 3-4 or 7-8, wherein the clostridial neurotoxin is administered by intradermal administration.

10. The method or the clostridial neurotoxin for use according to any one of claims 1-2, or 5-6, wherein the clostridial neurotoxin is administered to a patient having a level of said one or more sebaceous lipid, on an epidermal layer, that is lower than a level of said one or more sebaceous lipid on an epidermal layer of a subject that does not have the skin condition.

11. The method or the clostridial neurotoxin for use according to any one of claims 1-2, 5-6 or 10, wherein the skin condition is caused by a reduced level of said one or more sebaceous lipid, on an epidermal layer of the patient, compared to a level of said one or more sebaceous lipid on an epidermal layer of a subject that does not have the skin condition.

12. The method, the clostridial neurotoxin for use or non-therapeutic use according to any one of the preceding claims, wherein the clostridial neurotoxin is administered to a patient whose skin has been exposed to an agent that causes a decrease in the level of said one or more sebaceous lipid, on an epidermal layer of the patient, compared to a level of said one or more sebaceous lipid on an epidermal layer of a subject that has not been exposed to the agent.

13. The method, the clostridial neurotoxin for use or non-therapeutic use according to any one of the preceding claims, where said agent is a chemical selected from a metal, a chemical of soap, a fragrance, a preservative, a botanical chemical and paraphenylenediamine.

14. The method, the clostridial neurotoxin for use or non-therapeutic use according to any one of the preceding claims, wherein the clostridial neurotoxin is administered to a patient having sebum that comprises:
fatty acid at a concentration of <50% (v/v);
wax ester at a concentration of <20% (v/v);
squalene at a concentration of <10% (v/v); and/or
cholesterol at a concentration of <4% (v/v).

15. The method or the clostridial neurotoxin for use according to any one of claims 1-2, 5-6, or 10-14, wherein the skin condition is a condition associated with an aberrant sebaceous lipid level, wherein the sebaceous lipid is present on an epidermal layer at a lower level than on an epidermal layer of a patient that does not have the skin condition.

16. The method or the clostridial neurotoxin for use according to any one of claims 1-2, 5-6 or 10-15, wherein the skin condition is one or more condition selected from acne, atopic dermatitis, netherton syndrome, psoriasis, dehydrated skin (e.g. dry or cracked skin), actinic keratosis, rosacea, carbuncle, eczema, cellulitis, dermatitis, skin cancer and keratosis pilaris.

17. The method or the clostridial neurotoxin for use according to any one of claims 1-2, 5-6 or 10-16, wherein the skin condition is one or more condition selected from psoriasis, eczema, and dermatitis.

18. The method or the clostridial neurotoxin for use according to any one of claims 1-2, 5-6 or 10-17, wherein the skin condition is one or more selected from atopic dermatitis, psoriasis, xerosis, dehydrated skin (e.g. dry or cracked skin), eczema (e.g. atopic eczema), type 2 Gaucher disease, Sjögren-Larsson syndrome, ichthyosis (e.g. lamellar ichthyosis), X-linked ichthyosis, bullous ichthyosiform erythroderma, essential FFA deficiency, aged dry skin and hypohidrotic ectodermal dysplasia.

19. The method or the clostridial neurotoxin for use according to claim 18, wherein the skin condition is one or more selected from xerosis, psoriasis, atopic dermatitis, and ichthyosis; for example xerosis, psoriasis, or ichthyosis.

20. The method, the clostridial neurotoxin for use, or non-therapeutic use according to any one of the preceding claims, wherein the clostridium neurotoxin is administered to a non-facial area of the patient's skin.

21. The method, the clostridial neurotoxin for use, or non-therapeutic use according to any one of the preceding claims, wherein the clostridial neurotoxin is administered at the patient's hand(s), foot (e.g. feet), neck, scalp and/or back (e.g. upper back).

22. The method, the clostridial neurotoxin for use or non-therapeutic use according to any one of the preceding claims, wherein following administration of the clostridial neurotoxin:
no increase in the level of sebum on the skin of the patient is induced relative to a reference standard; and preferably no decrease in the level of sebum on the skin of the patient is induced relative to a reference standard;
wherein the reference standard corresponds to a level of sebum on the skin of a subject that has not been administered the clostridial neurotoxin, or wherein the reference standard corresponds to a level of sebum on the skin of the patient pre-administration of the clostridial neurotoxin.

23. The method, the clostridial neurotoxin for use or non-therapeutic use according to any one of the preceding claims, wherein the patient does not have oily skin prior to and/or subsequent to administration of the clostridial neurotoxin.

24. The method, the clostridial neurotoxin for use, or non-therapeutic use according to any one of the preceding claims, wherein the patient has sebum at a level of ≤180 mg/cm² of skin on the forehead, nasolabial sulcus, and/or nose.

25. The method, the clostridial neurotoxin for use, or non-therapeutic use according to any one of the preceding claims, wherein following administration to the patient, the clostridial neurotoxin induces retention of a tachykinin peptide in dermis of the skin.

26. The method, the clostridial neurotoxin for use, or non-therapeutic use according to claim 25, wherein said tachykinin peptide is Substance P (e.g. SEQ ID NO.: 17).

27. The method, the clostridial neurotoxin for use or non-therapeutic use according to any one of the preceding claims, wherein said clostridial neurotoxin is a BoNT/A neurotoxin.

28. The method, the clostridial neurotoxin for use or non-therapeutic use according to any one of the preceding claims, wherein said clostridial neurotoxin is a chimeric neurotoxin.

29. The method, the clostridial neurotoxin for use or non-therapeutic use according to claim 28, wherein the chimeric neurotoxin is selected from the group consisting of BoNT/DC and BoNT/X.

30. The method, the clostridial neurotoxin for use or non-therapeutic use according to claim 28, wherein the chimeric neurotoxin comprises a LH_{N} domain from a first neurotoxin covalently linked to a H_{C} domain from a second neurotoxin, preferably
wherein said first and second neurotoxins are different,
wherein the C-terminal amino acid residue of said LH_{N} domain corresponds to the first amino acid residue of the 3₁₀ helix separating the LH_{N} and H_{C} domains in said first neurotoxin, and
wherein the N-terminal amino acid residue of said H_{C} domain corresponds to the second amino acid residue of the 3₁₀ helix separating the LH_{N} and H_{C} domains in said second neurotoxin.

31. The method, the clostridial neurotoxin for use or non-therapeutic use according to claim 30, wherein said first neurotoxin is BoNT/A and wherein said second neurotoxin is BoNT/B.

32. The method, the clostridial neurotoxin for use or non-therapeutic use according to any one of the preceding claims, wherein the intradermal administration comprises administering a dose of the clostridial neurotoxin ranging from about 0.00025 ng-3 ng to the site of administration.

33. The method, the clostridial neurotoxin for use or non-therapeutic use according to any one of the preceding claims, wherein the intradermal administration comprises intradermal injection with a 30 gauge needle, preferably wherein the 30 gauge needle is inserted into dermis of the skin at an angle of about 5°-15° relative to a planar surface of the skin.

34. The method, the clostridial neurotoxin for use or non-therapeutic use according to any one of the preceding claims, wherein the epidermal layer is one or more selected from the Stratum basale, the Stratum spinosum, the Stratum granulosum, or the Stratum corneum.

35. The method, the clostridial neurotoxin for use or non-therapeutic use according to any one of the preceding claims, wherein the epidermal layer is the Stratum corneum.
